(19) United States
(12) Patent Application Publication
(10) Pub. No.: US 2010/0062441 A1
(43) Pub. Date: Mar. 11, 2010

(54) C-MET MUTATIONS AND USES THEREOF

(76) Inventor: Ravi Salgia, Park Ridge (IL)

Correspondence Address: Ballard Spahr LLP, SUITE 1000, 999 PEACHTREE STREET, ATLANTA, GA, 30309-3915, US

(21) Appl. No.: 12/530,172

(22) PCT Filed: Mar. 14, 2008
(86) PCT No.: PCT/US2008/057075

§ 371 Date: Sep. 4, 2009

Related U.S. Application Data
(60) Provisional application No. 60/918,254, filed on Mar. 15, 2007.

(51) Int. Cl. C12Q 1/68; C07K 14/00; C07H 21/04

(52) U.S. Cl. 435/6; 530/350; 536/23.1

(57) ABSTRACT

The present invention relates generally to the fields of molecular biology and growth factor regulation. More specifically, the invention concerns methods and compositions useful for diagnosing and treating human lung cancer associated with mutated c-Met.

### CROSS-REFERENCE TO RELATED APPLICATIONS

This application claims benefit of U.S. Provisional Application No. 60/918,254, filed Mar. 15, 2007. U.S. Provisional Application No. 60/918,254, filed Mar. 15, 2007 is hereby incorporated herein by reference in its entirety.

### BACKGROUND

Lung cancer is a growing concern around the world. Only 20% of all patients with non-small cell lung cancer (NSCLC) survive 5 or more years. NSCLC can be a heterogenous group of disorders, and can be subdivided into adenocarcinomas, bronchioloalveolar carcinoma, large cell carcinoma, and squamous cell carcinoma (SCC). These histologies can in themselves have different biological behavior as well as potentially different molecular signatures.

Head and neck cancer is the fifth most common cancer worldwide with an estimated global incidence of 500,000 new cases. Every year 40,000 Americans (2.8% of all cancers in the US) are diagnosed with head and neck cancer. 90-95% of head and neck cancers are of the squamous cell carcinoma (SCCHN) histology. Depending on stage, 35-40% of patients will expire as a consequence of their disease (Jemal et al., CA Cancer J Clin, 56, (2), 106-30 (2006)). The dominant causes of death are locoregional lymph node recurrences and metastatic spread. In particular the treatment options for metastatic disease remain inadequate and prognosis is dismal with an average life expectancy of 6-9 months. The advent of EGFR inhibitors, although beneficial for some patients, has not changed the overall prognosis of patients with recurrent disease.

Molecularly targeted therapies are increasingly at the center of interest for SCCHN. No significant progress has been made during the past 40 years for patients with recurrent disease except for the recent advent of EGFR inhibitors and the approval of cetuximab in March of 2006. Ten to twelve percent of patients will respond to an EGFR inhibitor (Trigo et al., 2004 ASCO Annual Meeting; Abstract No: 5502 (2004) and Cohen et al., J Clin Oncol, 21, (10), 1980-7 (2003)), but equally important 40% of patients will have stable disease. Furthermore EGFR inhibitors such as cetuximab or gefitinib have been successfully integrated into curative intent treatments of locally advanced tumors exemplified by studies by Bonner et al. (with radiotherapy) (Bonner et al., N Engl J Med, 354, (6), 567-78 (2006), and Cohen et al. (chemoradiotherapy) (Cohen et al., J Clin Oncol 2005; 23(suppl): abstract 5506 (2005)).

Epidermal growth factor receptor (EGFR) has been studied considerably, and therapeutic inhibition brought to clinical fruition. EGFR can sometimes be mutated (<10% in US population), amplified (preliminarily 25%), and overexpressed (50-70%) in NSCLC. The response to EGFR inhibition is 10-15% in NSCLC, and the response to small molecule inhibition is better seen with women, non-smokers, adenocarcinoma histologies, tumors with mutation or amplification of the EGFR gene, and in patients of Asian descent. Another targeted therapy against VEGF (bevacizumab) in combination with chemotherapy leads to enhancement of median overall survival by 2.3 months (Sandler et al., ASCO Proceedings 2005, Orlando, Fla. 2005, abstract: 4). To impact highly on NSCLC, further novel therapies must be identified.

The incidence of malignant melanoma has ALSO risen rapidly during the past several decades. In the year 2006, there will be 62,190 new cases of melanoma and 7,910 deaths making it the most common of all skin malignancies (Jemal et al., CA Cancer J Clin 56: 106-130 (2006). The majority of these deaths are due to distant metastases from the primary site since melanoma is notorious for its propensity to metastasize. Melanoma is poorly responsive to cytotoxic chemotherapy (Li et al., Oncogene, 20: 8125-35 (2001)) and survival of patients is based on screening, early detection and wide resection of the primary lesion. The overall survival for patients with metastatic melanoma ranges from only 4.7 to 11 months, with a median survival of 8.5 months (Sun et al., Curr Treat Options Oncol, 2: 193-202 (2001)). c-Met is over-expressed and associated with the metastatic potential of melanoma and patient survival (Slominski et al., Arch Pathol Lab Med 125: 1295-306 (2001); Barnhill et al., Semin Diagn Pathol, 10: 47-75 (1993); and Cruz et al., Oncology, 65: 72-82 (2003)).

The c-Met proto-oncogene was originally identified as the transforming oncogene in an osteosarcoma cell line that had been chemically mutagenized in vitro (generating the TPR-MET rearrangement with a potent tyrosine kinase). c-Met is overexpressed in a number of solid tumors, and expression correlates with an aggressive phenotype and poor prognosis (Maulik et al., Cytokine Growth Factor Rev, 13, (1), 41-59 (2002)). The contribution of c-Met overexpression and activation in the transformation of normal cells has recently been shown for osteoblasts (Patane et al., Cancer Res, 66, (9), 4750-7 (2006)). Overexpression of c-Met by lentiviral vector-mediated gene transfer resulted in the conversion of primary human osteoblasts into osteosarcoma cells, displaying the transformed phenotype in vitro and the distinguishing features of human osteosarcoma in vivo. The transformation and tumorigenesis were fully abrogated when c-Met expression was inhibited by short-hairpin RNA or by using dominant-negative c-Met. The role of c-Met and mutant c-Met in transforming normal human bronchial epithelial cells (NHBE) is currently unknown.

The gene that encodes for c-Met is located on chromosome 7, band 7q31, and spans more than 120 kb in length, consisting of 21 exons separated by 20 introns (FIG. 1) (Liu, Gene, 215, (1), 159-69 (1998); Seki et al., Gene, 102, (2), 213-9 (1991); and Maulik et al., Cytokine Growth Factor Rev, 13, (1), 41-59 (2002)). In mammalian cells the primary transcript produces a 150 kDa polypeptide that gets partially glycosylated, and produces a 170 kDa precursor protein. This is further glycosylated and then cleaved to produce a 50 kDa α-chain, and a 140 kDa β chain, which are then linked by disulfide bonds. The ligand for c-Met has been identified as hepatocyte growth factor (HGF). It was originally described as a potent mitogen for hepatocytes and later independently identified as scatter factor (SF), a secretory protein of fibroblasts and smooth muscle cells.

Signaling through the HGF/c-Met pathway has been demonstrated to trigger a variety of cellular responses that may vary based upon the cellular context. In vivo, HGF/c-Met signaling plays a role in growth, transformation of normal cells to malignant cells, cell motility, invasion, metastasis, epithelial to mesenchymal transition (EMT), angiogenesis, wound healing, and tissue regeneration.

The process of cell scattering can be divided into 3 phases: cell spreading, cell-cell dissociation, and cell migration. In order for epithelial cells to scatter, the disruption of cell-cell adhesions is required. Spontaneous cell scattering activity was shown in c-Met expressing NCI-H358 lung adenocarcinoma cell line by retroviral gene transduction of HGF (Yi et al., Neoplasia, 2, (3), 226-34 (2000)). In addition, the HGF overexpressing H358 cells show increased soft-agar colony formation and increased capacity to form xenograft tumors when implanted in the subcutaneous tissue of immune-deficient mice.

Cell motility comprises the formation and retraction of filopodia/lamellopodia, changes in actin formation, and cell migration. Studies show that HGF/c-Met signaling increases the motility of epithelial cells. Mutationally active Met induces the motility of Madin-Darby canine kidney cells (Jeffers et al., Proc Natl Acad Sci USA, 95, (24), 14417-22 (1998)). We demonstrated cell motility of SCLC is enhanced by HGF stimulation of c-Met RTK (Maulik et al., Clin Cancer Res, 8, (2), 620-7 (2002)). Cell motility is normally tightly controlled by PI3K, and GTPases, including Ras, Rac, and Rho (Nobes et al., Cell, 81, (1), 53-62 (1995)). PI3K appears to be an important molecule in HGF-induced mito-moto-, and morphogenesis, since inhibition of PI3K by wortmannin leads to decreased branching formation on a collagen matrix and chemotaxis of renal cells (Derman et al., Am J Physiol, 268, (6 Pt 2), F1211-7 (1995) and Derman et al., J Biol Chem, 271, (8), 4251-5 (1996)).

The mechanism by which HGF stimulation of c-Met leads to increased motility, migration, and invasion is not well understood. c-Met stimulation promotes cell movement, causes epithelial cells to disperse, and endothelial cells to migrate and promote chemotaxis (Maulik et al., Clin Cancer Res, 8, (2), 620-7 (2002)). Invasion is also mediated by c-Met signaling since mutant mice nullizygous for c-Met show that muscles originating from dermomyotome cells that migrate to the limb, diaphragm, and tip of the tongue fail to develop (Bladt et al., Nature, 376, (6543), 768-71 (1995)). With increased invasion, there is also increased metastasis seen in solid tumors (Maulik et al., Cytokine Growth Factor Rev, 13, (1), 41-59 (2002)).

HGF is the ligand for c-Met, and consists of 6 domains (N-terminal domain (n), four kringle domains (k1-k4), and a C-terminal domain (sp, structurally similar to the catalytic domain of serine proteinases)). There is a 2:2 stoichiometry of HGF binding to c-Met. HGF has been shown to bind to the sema domain (Gherardi et al., Proc Natl Acad Sci USA, 103, (11), 4046-51 (2006) and Stamos et al., Embo J, 23, (12), 2325-35 (2004)). The c-Met sema domain folds into a seven b-propeller structure, where blades 2 and 3 bottom face bind to HGF beta chain active site region. It would be useful to determine the binding of HGF to the various mutations of c-Met as well as crystallization of these motifs.

HGF binding to c-Met leads to phosphorylation of intracellular domain with a plethora of signal transduction cascade. Normally, activation of c-Met leads to receptor internalization into clathrin-coated vesicles, delivery to sorting endosomes, and degradation via the lysosomal pathway (Teis and Huber, Cell Mol Life Sci, 60, (10), 2020-33 (2003); Hammond et al, Oncogene, 20, (22), 2761-70 (2001)). Monoubiquitination of c-Met is important for trafficking and targeting for lysosomal degradation (Abella et al., Mol Cell Biol, 25, (21), 9632-45 (2005)). c-Cbl is a E3-ubiquitin ligase that monoubiquinates the c-Met receptor, thereby directing internalization, trafficking to late endosomes, and ultimate degradation. c-Cbl also regulates endocytosis by acting as an adaptor for endophilin, an enzyme involved in membrane curvature (Soubeyran et al., Nature, 416, (6877), 183-7 (2002)).

Monoubiquitinated c-Met interact with multiple proteins of the endocytic pathway that contain ubiquitin-interacting domains, especially Hrs (HGF-regulated tyrosine kinase substrate). Hrs is believed to be involved in the retention of ubiquitinated receptors within the bilayered clathrin coat and in the recruitment of endosomal sorting complex required for transport complexes (Bache et al., J Biol Chem, 278, (14), 12513-21 (2003)). Hrs is tyrosine phosphorylated in response to HGF stimulation, and required for internalization of c-Met. Abella et al, have shown that a ubiquitination-deficient c-Met (Y1003F, in the JM domain) shows increased stability of c-Met (i.e. decreased receptor degradation and thus further recycling to the membrane) and signaling of downstream pathways, and oncogenic activation in vivo (Abella et al., Mol Cell Biol, 25, (21), 9632-45 (2005)). This has large implications for the JM domain mutants identified in the NSCLC samples. Activation of these tumors could be due to potentially defective receptor cycling.

Overexpression of c-Met has been found in Small Cell Lung Cancer (SCLC) and NSCLC cells (Olivero et al., Br J Cancer, 74, (12), 1862-8 (1996) and Ichimura et al., Jpn J Cancer Res, 87, (10), 1063-9 (1996)). c-Met receptor expression seems to be associated with higher pathological tumor stage and worse outcome. Ichimura et al. reported that Met was overexpressed in all eleven NSCLC cell lines studied, and in 34 of 47 adenocarcinomas and 20 of 52 SCC (Ichimura et al., Jpn J Cancer Res, 87, (10), 1063-9 (1996)). Siegfried et al. showed that the expression of Met was 2 to 10-fold higher than in adjacent normal lung tissue in 25% of NSCLC tumors, and HGF levels were 10 to 100-fold higher in carcinoma samples as compared to adjacent normal tissue (Siegfried et al., Cancer Res, 57, (3), 433-9) (1997)). Higher levels of HGF were associated with more aggressive biology and a poorer prognosis in NSCLC (Siegfried et al., Ann Thorac Surg, 66, (6), 1915-8 (1998)). It was postulated that HGF, produced by mesenchymal cells within the tumor, acts upon epithelial cells that express its receptor, Met, thus representing a paracrine activation loop (Tokunou et al., Am J Pathol, 158, (4), 1451-63 (2001)). HGF overexpressing transgenic mice have been shown to be more susceptible to carcinogenic induced lung cancer (Stabile et al., Carcinogenesis, 27, (8), 1547-55 (2006)). In large cell neuroendocrine carcinoma of lung, c-Met was strongly expressed in 47% of tumor tissues, and significantly correlated with survival in a univariate analysis (Rossi et al., J Clin Oncol, 23, (34), 8774-85 (2005)).

c-Met is a RTK that was postulated by multiple authors to be an important molecule in the pathogenesis and metastasis of SCCHN and has been shown to be highly expressed in SCCHN (Marshall, et al. Laryngoscope, 108, (9), 1413-7 (1998); Lo Muzio, et al., Tumour Biol, 27, (3), 115-21 (2006); and Chen, et al. Taiwan. J Oral Pathol Med, 33, (4), 209-17 (2004)). Furthermore, activating c-Met mutations in 10-25% of SCCHN were described by two groups both implicating the tyrosine kinase domain (Aebersold, et al. Oncogene, 22, (52), 8519-23 (2003); Cortesina, et al., Int J Cancer, 89, (3), 286-92 (2000); (Morello, et al., J Cell Physiol, 189, (3), 285-90, (2001); and Di Renzo, et al., Oncogene, 19, (12), 1547-55 (2000)). In particular the Y1253D mutation was described in 10% of tumors. In addition, Di Renzo reports that mutations seem to be selected for in metastasis, whereas mutations are more difficult to detect in primary tumor tissues due to lower frequency. This was interpreted as particular contribution of c-Met mutations to the metastatic process in head and neck squamous cell carcinomas.

c-Met is normally expressed by epithelial cells and has been found to be overexpressed and amplified in a variety of human tumor tissues (Maulik, et al., Clin Cancer Res., 8, (2), 620-7, (2002); Olivero, et al., Br J Cancer, 74, (12), 1862-8, (1996); Natali, et al., Int J Cancer, 69, (3), 212-7, (1996); Porte, et al., Clin Cancer Res., 4, (6), 1375-82, (1998); Ramirez, et al., Clin Endocrinol (Oxf 53, (5), 635-44,) (2000); Hellman, et al., Cancer Cell 1, (1), 89-97, (2002); Ferracini, et al., Oncogene, 10, (4), 739-49 (1995); and Di Renzo, et al., Clin Cancer Res 1, (2), 147-54, (1995)), (Di Renzo, et al., Int J Cancer, 58, (5), 658-62 (1994)). Many missense mutations of c-Met were reported in a variety of cancers, with the majority of them identified in the cytoplasmic activation-loop tyrosine kinase domain (Jeffers, et al., Proc Natl Acad Sci USA, 95, (24), 14417-22 (1998)) (FIG. 1). Identification of activating mutations of c-Met in hereditary papillary renal carcinomas provided the first direct evidence linking c-Met directly to human oncogenesis (Schmidt, et al., Nat Genet, 16, (1), 68-73 (1997)). Germline missense mutation in the tyrosine kinase domain are detected in the majority of hereditary papillary renal cell carcinomas (HPRCC) (Schmidt, et al., Cancer Res, 58, (8), 1719-22 (1998)); whereas somatic mutations have been found in some sporadic papillary renal carcinomas (Schmidt, et al., Oncogene, 18, (14), 2343-50 (1999)). Liao et al have identified specific gain-of-function germline mutation of c-Met (G966S, juxtamembrane domain, frequency of 74%) in Rottweiler dogs specifically (Liao et al. Anim Genet, 37, (3), 248-52 (2006)). Rottweilers are believed to be predisposed to certain cancers, including osteosarcoma, lymphoma, and histiocytic sarcoma.

Tyrosine kinase domain mutations have been described for SCCHN, particularly in lymph node metastases. Studies by Comoglio et al. revealed that primary tumors did not harbor any c-Met mutations or were only detectable at very low levels, however, there were mutations in the lymph node metastases in 25% of tumors (Di Renzo, et al., Oncogene 19, (12), 1547-55, (2000)). In another report 10.9% of tumors harbored the Y1253D mutation, but again only detectable at low levels with sensitive techniques in the primary tumor (Aebersold, et al., Oncogene, 22, (52), 8519-23 (2003) and Di Renzo, et al., Oncogene 19, (12), 1547-55, (2000))

Besides missense mutations, c-Met-mediated tumorigenesis could be a result of gene amplification, as seen in human gastric carcinoma via the break-fusion-bridge (BFB) mechanism (Hellman et al., Cancer Cell 1, (1), 89-97 (2002)). Approximately 10-20% of gastric carcinomas can have c-Met amplification (Sakakura et al., Genes Chromosomes Cancer, 24, (4), 299-305 (1999)). Smolen et al have shown that amplified c-Met gastric cancer cell lines are more susceptible to c-Met inhibition with PHA665752 (Smolen et al., Proc Natl Acad Sci USA, 103, (7), 2316-21 (2006))

JM (juxtamembrane) domains of RTKs are thought to be key regulators of catalytic functions. More recently, the structural basis of the regulatory role (auto-inhibition) of the RTK Eph-B2 by the unphosphorylated JM domain has been elucidated (Wybenga-Groot, et al., Cell 106, (6), 745-57 (2001)). A germline mutation P1009S (exon 14) of c-Met was detected in a patient with gastric carcinoma and is the first such missense mutation to be described affecting the JM domain (as opposed to tyrosine kinase domain). The P1009S mutation does not induce ligand-independent activation of c-Met, but showed increased persistent response to HGF stimulation when expressed in NIH3T3 cells (Lee, et al., Oncogene, 19, (43), 4947-53 (2000)). Peschard et al. have shown that the c-Cbl acts as a negative regulatory protein for c-Met, as well as several other RTKs, by promoting the poly-ubiquitination of c-Met (Peschard, et al., Mol Cell, 8, (5), 995-1004 (2001)). The Y1003 JM tyrosine, when replaced by phenylalanine (Y1003F), resulted in the loss of ubiquitination of the Met receptor and transformed activity in fibroblast and epithelial cells.

Recently, specific JM mutations of c-Met (R988C and T1010I) in SCLC were described (See Ma, et al. Cancer Res 65, 1479-88 (2005) and WO 2006/104912). These studies also described that a study of 127 adenocarcinoma NSCLC tumors revealed that there were germline mutations of c-Met at R988C, T1010I, and S1058P. These JM domain mutations of c-Met led to enhanced tumorigenicity, increased cell motility, altered cellular architecture, increased c-Met and downstream signal molecule phosphorylation, and stronger response to therapeutic inhibition with small molecule inhibitors (Ma, et al. Cancer Res, 65, (4), 1479-88 (2005) and Jagadeeswaran, et al., Cancer Res 66, (1), 352-61 (2006)). In these studies, it was shown from 126 patients with adenocarcinomas that R988C and T1010I (and T1010A) were germline mutations. It is possible that these variations may affect lung cancer risk in carriers. Furthermore, the R988C variation in c-Met was shown to be important in lung tumorigenesis of SWR/J mouse strain (with Par4-susceptible allele) (Zaffaroni, et al., Oncogene, 24, (6), 1084-90 (2005))

The sema-domain is conserved among all semaphorins and is also found present in the plexins and c-Met (Tessier-Lavigne et al., Science, 274, (5290), 1123-33, (1996); Tamagnone, et al Cell, 99, (1), 71-80 (1999)); and Christensen, et al., Cancer Res, 58, (6), 1238-44 (1998)). In c-Met, the sema domain is encoded by exon 2, and binds specifically to HGF. More recently, the 3D conformation of the HGF and heparin-binding sites of c-Met have been established by deletion mutagenesis of the RTK (Kong-Beltran et al., Cancer Cell, 6, (1), 75-84 (2004)). The extracellular ligand-binding domain in the c-Met ectodomain was identified as adopting a seven-bladeβ-propeller fold for the sema domain of c-Met, homologous to the β-propeller fold template seen in the N-terminal domain of αᵥ-integrin. These 3D models and functional map of the c-Met ectodomain would undoubtedly facilitate further development of targeted therapeutics against c-Met (Tessier-Lavigne et al., Science, 274, (5290), 1123-33, (1996); Tamagnone, et al Cell, 99, (1), 71-80 (1999); and Christensen, et al., Cancer Res, 58, (6), 1238-44 (1998)). Unique mutations in the sema domain from SCLC and NSCLC samples are described herein.

Recently, it has been determined that mutations in the EGFR tyrosine kinase domain can occur in adenocarcinomas, with a frequency of <10% in U.S. population and 40-55% in East Asian populations (such as Japanese and Taiwanese) (Kosaka, et al., Cancer Res, 64, (24), 8919-23 (2004) and Paez, et al., Science, 304, (5676), 1497-500 (2004)). EGFR mutations do not occur in tumors with KRAS2 mutations in adenocarcinomas, whereas EGFR mutations were independent of TP53 mutations (Kosaka, et al., Cancer Res, 64, (24), 8919-23 (2004)). RAS oncogene is also involved in lung cancer development. Of the three human RAS genes (HRAS, NRAS, and KRAS), RAS mutations are detected in 30% of adenocarcinomas. The majority of RAS mutations are in the KRAS2 gene (>90%), and 80% of KRAS2 mutations occur in codon 12 (Rodenhuis, et al., Cancer Res, 48, (20), 5738-41 (1988)). Other mutations are located in codons 13 and 61, and the predominant mutation is a G-T transversion (Rodenhuis et al., Am Rev Respir Dis, 142, (6 Pt 2), S27-30 (1990)). The point mutation leads to inactivation of intrinsic GTPase activity of RAS, thereby leading to proliferation (Rodenhuis, et al., Semin Cancer Biol, 3, (4), 241-7 (1992)).

Unlike imatinib for CML (targeting Bcr/Abl) and gastrointestinal stromal tumors (GIST; targeting c-Kit), targeted small molecule inhibitors against c-Met have not come to clinical fruition yet. Several c-Met inhibitors are currently in clinical development. Also an antagonist of HGF, NK4, was previously reported to be generated by proteolytic digestion of HGF Date, et al., FEBS Lett, 420, (1), 1-6 (1997)). NK4 is a truncated HGF composed of the NH2-terminal hairpin domain and four kringle domains in the alpha-chain of HGF. It retains c-Met receptor binding properties without mediating biological responses. NK4 antagonizes HGF-induced tyrosine phosphorylation of c-Met, resulting in inhibition of HGF-induced motility and invasion of HT115 human colorectal cancer cells, as well as angiogenesis (Parr, et al., Int J Cancer, 85, (4), 563-70 (2000)). Also, when administered to pancreatic tumor-bearing mice, NK4 inhibited growth, invasion, and disseminating metastasis of pancreatic cancer cells and prolonged the lifespan of these mice (Tomioka, et al., Cancer Res, 61, (20), 7518-24 (2001)). Finally, a soluble chimeric form of c-Met was shown to retain full capacity to bind HGF and therefore neutralize HGF activity Mark, et al., J Biol Chem, 267, (36), 26166-71 (1992)). NK4, pro-HGF (uncleavable HGF) and the decoy c-Met receptor have been shown to inhibit mutant c-Met-induced transformation of NIH3T3 cells (Michieli, et al., Oncogene, 18, (37), 5221-31 (1999)).

Small molecule inhibitors directed specifically against c-Met represent an attractive novel targeted therapeutic approach. The effectiveness of a novel small molecule specific inhibitor of c-Met, SU11274 was first reported by Sattler, et al. (Pfizer; previously Sugen), in cells transformed by the oncogenic Tpr-Met as a model, as well as in SCLC (Sattler, et al., Cancer Res, 63, (17), 5462-9 (2003)). Inhibition of the Met kinase activity by the drug SU11274 led to time- and dose-dependent reduced cell growth and induced G1 cell cycle arrest and apoptosis (Ma, et al., Cancer Res, 65, (4), 1479-88 (2005)). Met kinase autophosphorylation was reduced on sites that have been previously shown to be important for activation of pathways involved in cell growth and survival, especially the phosphatidylinositol-3′-kinase (PI3K) and the Ras pathway. The characterization of SU11274 as an effective inhibitor of Met tyrosine kinase activity illustrates the therapeutic potential of targeting Met in cancers associated with activated forms of this kinase.

Small interference RNA (siRNA) is another novel approach to study the effects of inhibition on RTKs. In lung cancer CL-1 cells, siRNA has been used to inhibit the membrane-anchored glycoprotein RECK and it has been shown that histone-deacetylase inhibitors suppress the tumor invasion with their inhibitory effect on MMP-2 activation mediated via RECK (Liu, et al. Cancer Res, 63, (12), 3069-72 (2003)). However, this novel inhibitory strategy used against c-Met has not been reported in lung or head and neck cancer. This would be another promising and powerful tool to utilize in dissecting the effects of c-Met and its mutations on the tumor biology in lung or head and neck cancer.

To impact on this disease, newer and novel targeted therapies need to be employed. As described herein, it has been determined that c-Met is expressed, functional, and sometimes mutated or amplified in NSCLC as well as SCCHN. Also described herein are unique mutations of c-Met in the semaphorin, juxtamembrane, and tyrosine kinase domain of c-Met.

Even with the best therapies and recent advent of novel molecularly targeted therapies, overall survival for all Non-Small Cell Lung Cancer (NSCLC) patients is only 20% over a five year period. Receptor tyrosine kinases (RTKs) have shown to be important in a variety of malignancies, such as c-Kit in GISTs and epidermal growth factor receptor (EGFR) in NSCLC. However, the response to EGFR blockade by small molecule inhibitors, such as erlotinib, is at best 5-15% in refractory advanced NSCLC. The compositions and methods disclosed herein will provide a means to address such issues.

All references cited herein, including patent applications and publications, are incorporated by reference in their entirety.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. These are non-limiting examples.

FIG. 1 shows the predicted structure and functional domains of c-Met. The figure shows the functional domains of c-Met: the Sema domain (Semaphorin-like), the PSI domain (found in plexins, semaphorins, and integrins), the IPT repeat domains (found in Ig-like regions, plexins and transcription factors), the Trans-membrane (TM) domain, Juxta-membrane (JM) domain, and the Tyrosine Kinase (TK) domain (located intracellularly). Various serine and tyrosine phosphorylation sites important for cellular functions are also shown.

FIG. 2 shows the predicted structure and functional domains of c-Met as well as the various mutations identified in each region. (A) The figure shows the functional domains of c-Met: the Sema domain (Semaphorin-like), the PSI domain (found in plexins, semaphorins, and integrins), the IPT repeat domains (found in Ig-like regions, plexins and transcription factors), the Trans-membrane (TM) domain, Juxta-membrane (JM) domain, and the Tyrosine Kinase (TK) domain (located intracellularly). (B) shows various c-Met mutations as well as what the types of cancers fromin which the mutations have been identified. Non-small cell lung carcinoma (NSCLC); small lung cell carcinoma (SCLC); malignant pleural mesothelioma (MPM); head and neck squamous cell carcinoma (HNSCC); melanoma; hepatocellular carcinoma (HCC); Glioma; Breast; gastric and renal cell carcinoma (RCC).

FIG. 3 shows c-Met structure, mutations and function. (A) shows c-Met domain structure and frequency of c-met mutations among ethnic groups. Synonymous mutations are italicized. Mutation frequencies are expressed as a percentage of total number of tumor samples in each ethnic group. (B) shows an overview of c-Met missense mutations on sema domain of c-Met and HGF β-chain complex by homology modeling. (i) Ribbon representation with HGF β-chain shown in yellow and sema domain of c-Met in purple colors. Surface representation of mutated residues are colored red and labeled by residue number. Residues 168 and 229 can be seen in direct contact with HGF. (ii) Van der Waals (VDW) or space filling spheres representation of positively selected residues P169, S170, and P210, with high probabilities of ω>1, are spatially close to mutation E168D, indicating residue 168 in potential functional region. (iii) Stereo magnification of the mutation N375S, N375 (left) has two potential hydrogen bonds (green dash line), while 5375 modeling structure (right) lost one hydrogen bond. (C) shows prolonged activation of c-Met signaling in c-Met mutants. Cos7 cells stably transfected with vector control, wild-type c-Met, and c-Met mutants E168D and L229F were serum starved for 2 h followed by stimulation with HGF. HGF-c-Met signaling at various time intervals was estimated by immunoblotting of the cell lysates with the following antibodies: p-Met [Y1003] gel (1), p-Met [Y1230/1234/1235] gel (2), c-Met (gel 3), p-AKT [S473] (gel 4), AKT (gel 5), and b-actin (gel 6).

FIG. 4 shows inhibition of c-Met with small molecule specific c-Met inhibitor (compound X, a third generation inhibitor) in NSCLC cells. (A) shows the cellular kinase activities were measured using ELISA capture method. Compound X showed a high probability of c-Met and ALK inhibition at clinically relevant doses. (B) shows c-Met Amplified H1993 or over expressed A549 cells and c-Met null H522 were plated and treated with the indicated concentration of the c-Met inhibitor. Cell viability and growth inhibitory effect of c-Met inhibitors was assayed using MTT assay and Colony formation assay in triplicates.

FIG. 5 shows a summary of missense mutations of c-Met in lung cancer tissues of different ethnic groups.

FIG. 6 shows computational analysis of missense mutations in sema domain of c-Met.

FIG. 7 shows EGFR tyrosine kinase domain mutation characteristics.

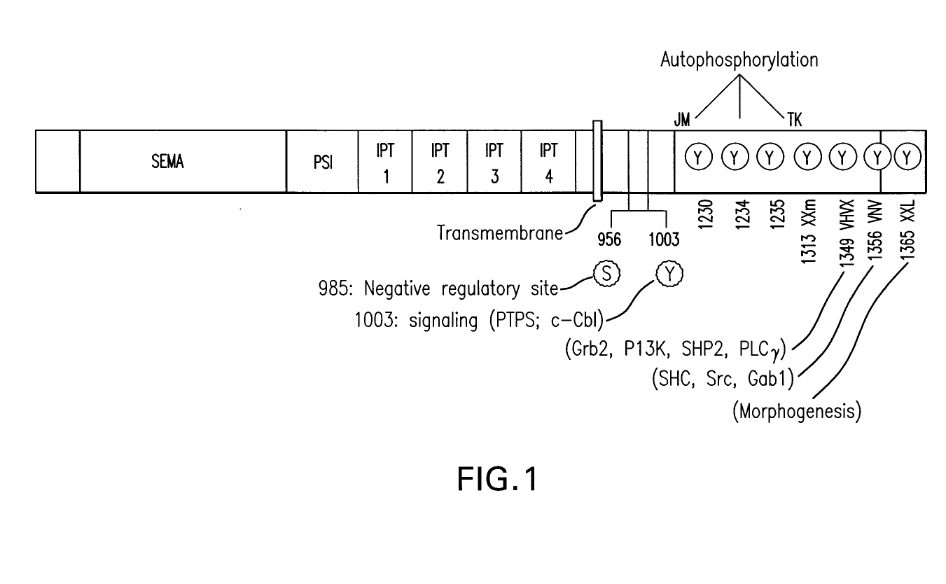

### DETAILED DESCRIPTION OF THE INVENTION

The present disclosure describes, at least in part, the discovery of multiple mutational events in the receptor of human hepatocyte growth factor (HGF), c-Met, that are closely associated with tumorigenesis. It was previously thought that aberrant c-Met activity was associated with various cancers, however, it was unknown what, if any, specific mutations resulted in dysregulation of the c-Met signaling pathway. In particular, it was not clear what, if any, mutations outside of the kinase domains are associated with the development of human tumors, e.g. lung tumors, or head and neck tumors. Disclosed herein are examples of mutational events in the different domains of c-Met, including but not limited to the sema, juxtamembrane and tyrosine kinase domains of c-Met that are found in human tumors. It is believed that these mutations predispose and/or directly contribute to human tumorigenesis. Indeed, as described herein, some of the mutations directly affect the c-Met protein structure.

All patents, patent applications and publications cited herein, whether supra or infra, are hereby incorporated by reference in their entireties into this application in order to more fully describe the state of the art as known to those skilled therein as of the date of the invention described and claimed herein.

It is to be understood that this invention is not limited to specific synthetic methods, or to specific recombinant biotechnology methods unless otherwise specified, or to particular reagents unless otherwise specified, to specific pharmaceutical carriers, or to particular pharmaceutical formulations or administration regimens, as such may, of course, vary. It is also to be understood that the terminology used herein is for the purpose of describing particular embodiments only and is not intended to be limiting.

### DEFINITIONS AND NOMENCLATURE

The terminology used herein is for the purpose of describing particular embodiments only and is not intended to be limiting.

The invention relates to the discovery that cancer is caused by mutations in the gene c-met. c-met refers to the gene (or other nucleic acid) encoding a c-Met polypeptide. c-Met refers to the polypeptide encoded by a c-met gene. Both of these terms are used herein as general identifiers. Thus, for example, a c-met gene or nucleic acid refers to any gene or nucleic acid identified with or derived from a wild-type c-met gene. For example, a mutant c-met gene is a form of c-met gene.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “reverse analog” or “reverse sequence” refers to a peptide having the reverse amino acid sequence as another, reference, peptide. For example, if one peptide has the amino acid sequence ABCDE, its reverse analog or a peptide having its reverse sequence is as follows: EDCBA.

By “increased susceptibility to develop a cancer” is meant a subject who has a greater than normal chance of developing a cancer, compared to the general population. Such subjects could include, for example, a subject that harbors a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By “modulate” is meant to alter, by increase or decrease.

By “normal subject” is meant an individual who does not have an increased susceptibility for developing a cancer.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

By a “transgene” is meant a nucleic acid sequence that is inserted by artifice into a cell and becomes a part of the genome of that cell and its progeny. Such a transgene may be (but is not necessarily) partly or entirely heterologous (for example, derived from a different species) to the cell.

By “transgenic animal” an animal comprising a transgene as described above. Transgenic animals are made by techniques that are well known in the art.

By “knockout mutation” is meant an alteration in the nucleic acid sequence that reduces the biological activity of the polypeptide normally encoded therefrom by at least 80% relative to the unmutated gene. The mutation may, without limitation, be an insertion, deletion, frameshift, or missense mutation. A “knockout animal,” for example, a knockout mouse, is an animal containing a knockout mutation. The knockout animal may be heterozygous or homozygous for the knockout mutation. Such knockout animals are generated by techniques that are well known in the art. A preferred form of knockout mutation is one where the biological activity of the c-Met polypeptide is not completely eliminated.

By “treat” is meant to administer a compound or molecule of the invention to a subject, such as a human or other mammal (for example, an animal model), that has an increased susceptibility for developing a cancer, or that has a cancer, in order to prevent or delay a worsening of the effects of the disease or condition, or to partially or fully reverse the effects of the disease.

By “prevent” is meant to minimize the chance that a subject who has an increased susceptibility for developing a cancer will develop a cancer.

By “specifically binds” is meant that an antibody recognizes and physically interacts with its cognate antigen (for example, a c-Met polypeptide) and does not significantly recognize and interact with other antigens; such an antibody may be a polyclonal antibody or a monoclonal antibody, which are generated by techniques that are well known in the art.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for c-Met nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the c-Met nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

By “familial mutation” or “inherited mutation” is meant a mutation in an individual that was inherited from a parent and that was present in somatic cells of the parent. By “sporadic mutation” or “spontaneous mutation” is meant a mutation in an individual that arose in the individual and was not present in a parent of the individual.

As set forth herein, nucleotides are numbered according to the cDNA sequence for c-Met (SEQ ID NO: 1).

The nucleotide and amino acid sequence of c-Met are shown in SEQ ID NO:1 and SEQ ID NO:2, respectively, starting at nucleotide 1 and amino acid 1, respectively.

As used herein, a specific notation will be used to denote certain types of mutations. All notations referencing a nucleotide or amino acid residue will be understood to correspond to the residue number of the wild-type c-Met nucleic acid sequence set forth at SEQ ID NO:1, or of the wild-type c-Met polypeptide sequence set forth at SEQ ID NO:2. Thus, for example, the notation “G504T” when used in the context of a nucleotide sequence will be used to indicate that the nucleotide G at position 504 of the sequence set forth at SEQ ID NO:1 has been replaced with a T. Similarly, the notation “E168D” when used in the context of a polypeptide sequence will be used to indicate that the amino acid Glutamate at position 168 has been replaced with Aspartate.

In the method of the invention, the mutant c-Met polypeptide or mutated c-Met nucleic acid identified is associated with cancers.

### Compositions

The disclosed compositions are related to the receptor of the hepatocyte growth factor (HGF), c-Met. Disclosed herein are compositions, such as polynucleotides capable of specifically hybridizing to c-Met encoding nucleic acid comprising a mutation at a nucleic acid position corresponding to a change in amino acid at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed are polynucleotides capable of specifically hybridizing to c-Met encoding nucleic acid that comprises a mutation in a sequence that encodes exons 2, 3, 4, 7, 9, 13, 14, 15, 17, 19, 20 and/or 21. For example, disclosed are isolated polynucleotides capable of encoding polypeptides comprising a mutation at a nucleic acid position corresponding to an amino acid change of SEQ ID NO: 2 at positions L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I, or a complement thereof.

Also disclosed are isolated polynucleotides that comprise mutations in a nucleotide sequence capable of encoding a c-Met protein, that do not result in a change in the amino acid sequence. Such mutations can sometimes be referred to as “silent mutations”. For example, disclosed are isolated polynucleotides comprising a mutation at a nucleic acid position corresponding to the amino acids at positions A48A, S178S, Q648Q, I706I, K1250K, D1304D, A1357A, P1382P, I377I, R1184R, or a complement thereof. The “silent mutations” described above, can be used in the same methods and within the same compositions as the other mutations described herein.

The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

The nucleotides of the invention can comprise one or more nucleotide analogs or substitutions. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (ψ), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙ O]ₘ CH₃, —O(CH₂)ₙ OCH₃, —O(CH₂)ₙ NH₂, —O(CH₂), CH₃, —O(CH₂)ₙ —ONH₂, and —O(CH₂)ₙON[(CH₂)ₙ CH₃)]2, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N3, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety for their teaching of modifications and methods related to the same.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety for their teaching of modifications and methods related to the same.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety or sugar moieties replaced. Nucleotide substitutes do not contain a standard phosphorus atom. Substitutes for the phosphate can be, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety for their teaching of modifications and methods related to the same.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference in its entirety for their teaching of modifications and methods related to the same. (See also Nielsen et al., Science, 254, 1497-1500 (1991)).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Aim. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety for their teaching of modifications and methods related to the same.

The same methods of calculating homology as described elsewhere herein concerning polypeptides can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

Also, disclosed are compositions including primers and probes, which are capable of interacting with the polynucleotide sequences disclosed herein. For example, disclosed are primers/probes capable of amplifying a nucleic acid encoding human c-Met, wherein the nucleic acid comprises a mutation that results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Examples of such primers/probes are disclosed in Tables 7-10.

The disclosed primers can used to support DNA amplification reactions. Typically the primers will be capable of being extended in a sequence specific manner. Extension of a primer in a sequence specific manner includes any methods wherein the sequence or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the polynucleotide sequences disclosed herein or region of the polynucleotide sequences disclosed herein or they hybridize with the complement of the polynucleotide sequences disclosed herein or complement of a region of the polynucleotide sequences disclosed herein.

The size of the primers or probes for interaction with the polynucleotide sequences disclosed herein in certain embodiments can be any size that supports the desired enzymatic manipulation of the primer, such as DNA amplification or the simple hybridization of the probe or primer. A typical primer or probe would be at least 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long or any length inbetween.

Also disclosed are functional nucleic acids that can interact with the disclosed polynucleotides. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of polynucleotide sequences disclosed herein or the genomic DNA of the polynucleotide sequences disclosed herein or they can interact with the polypeptide encoded by the polynucleotide sequences disclosed herein. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Disclosed herein are antisense molecules that interact with the disclosed polynucleotides. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437 each of which is herein incorporated by reference in its entirety for their teaching of modifications and methods related to the same.

Also disclosed are aptamers that interact with the disclosed polynucleotides. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of aptamers, the background protein could be ef-1α. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. Also disclosed are ribozymes that interact with the disclosed polynucleotides.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Also disclosed are triplex forming functional nucleic acid molecules that interact with the disclosed polynucleotides. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

Also disclosed are external guide sequences that form a complex with the disclosed polynucleotides. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

Also disclosed are polynucleotides that contain peptide nucleic acids (PNAs) compositions. PNA is a DNA mimic in which the nucleobases are attached to a pseudopeptide backbone (Good and Nielsen, Antisense Nucleic Acid Drug Dev. 1997; 7(4) 431-37). PNA is able to be utilized in a number of methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 Jun.; 15(6):224-9). As such, in certain embodiments, one may prepare PNA sequences that are complementary to one or more portions of an mRNA sequence based on the disclosed polynucleotides, and such PNA compositions may be used to regulate, alter, decrease, or reduce the translation of the disclosed polynucleotides transcribed mRNA, and thereby alter the level of the disclosed polynucleotide's activity in a host cell to which such PNA compositions have been administered.

PNAs have 2-aminoethyl-glycine linkages replacing the normal phosphodiester backbone of DNA (Nielsen et al., Science Dec. 6, 1991; 254(5037):1497-500; Hanvey et al., Science. Nov. 27, 1992; 258(5087):1481-5; Hyrup and Nielsen, Bioorg Med. Chem. 1996 Jan.; 4(1):5-23). This chemistry has three important consequences: firstly, in contrast to DNA or phosphorothioate oligonucleotides, PNAs are neutral molecules; secondly, PNAs are achirial, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med. Chem. 1995 Apr.; 3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med. Chem. 1995 Apr.; 3(4):437-45; Petersen et al., J Pept Sci. 1995 May-Jun.; 1(3):175-83; Orum et al., Biotechniques. 1995 Sep.; 19(3):472-80; Footer et al., Biochemistry. Aug. 20, 1996; 35(33): 10673-9; Griffith et al., Nucleic Acids Res. Aug. 11, 1995; 23(15):3003-8; Pardridge et al., Proc Natl Acad Sci USA. Jun. 6, 1995; 92(12):5592-6; Boffa et al., Proc Natl Acad Sci USA. Mar. 14, 1995; 92(6):1901-5; Gambacorti-Passerini et al., Blood. Aug. 15, 1996; 88(4):1411-7; Armitage et al., Proc Natl Acad Sci USA. Nov. 11, 1997; 94(23):12320-5; Seeger et al., Biotechniques. 1997 Sep.; 23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993; 65(24):3545-9) and Jensen et al. (Biochemistry. Apr. 22, 1997; 36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Also disclosed is an isolated polynucleotide capable of distinguishing between an isolated polynucleotides capable of encoding polypeptides comprising a mutation at a nucleic acid position corresponding to an amino acid change of SEQ ID NO: 2 at positions L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I or a complement thereof and a nucleic acid encoding a wild type Met receptor tyrosine kinase protein.

Optionally, isolated polypeptides or isolated nucleotides can also be purified, e.g., are at least about 90% pure, more preferably at least about 95% pure and most preferably at least about 99% pure.

An “isolated” polypeptide or an “isolated” polynucleotide is one that is removed from its original environment. For example, a naturally-occurring polypeptide or polynucleotide is isolated if it is separated from some or all of the coexisting materials in the natural system.

Also disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of molecules including the polypeptide are discussed, specifically contemplated is each and every combination and permutation of polypeptide and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that one way to define any known variants and derivatives or those that might arise, of the disclosed genes and proteins herein is through defining the variants and derivatives in terms of homology to specific known sequences. For example SEQ ID NO: 1 sets forth a particular sequence of the wild-type c-met gene and SEQ ID NO: 2 sets forth a particular sequence of the protein encoded by SEQ ID NO: 1, the receptor of the hepatocyte growth factor (HGF), c-Met. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference in its entirety and at least for material related to hybridization of nucleic acids). As used herein “stringent hybridization” for a DNA:DNA hybridization is about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein. Optionally, one or more of the isolated polynucleotides of the invention are attached to a solid support. Solid supports are disclosed herein.

Also disclosed herein are arrays comprising polynucleotides capable of specifically hybridizing to c-Met encoding nucleic acid comprising a mutation at a nucleic acid position corresponding to a change in amino acid at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed are arrays comprising polynucleotides capable of specifically hybridizing to c-Met encoding nucleic acid that comprises a mutation in a sequence that encodes exons 2, 3, 4, 7, 9, 13, 14, 15, 17, 19, 20 and/or 21.

Also disclosed herein are solid supports comprising one or more polypeptides selected from the group consisting of SEQ ID NOs: 3-26 attached to the solid support. Additionally disclosed are solid supports comprising one or more polynucleotides capable of encoding one or more polypeptides selected from the group consisting of SEQ ID NOs: 3-26.

Solid supports are solid-state substrates or supports with which molecules, such as analytes and analyte binding molecules, can be associated. Analytes, such as calcifying nano-particles and proteins, can be associated with solid supports directly or indirectly. For example, analytes can be directly immobilized on solid supports. Analyte capture agents, such a capture compounds, can also be immobilized on solid supports. For example, disclosed herein are antigen binding agents capable of specifically binding to a c-Met polypeptide comprising a mutation at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed is an antigen binding agent capable of specifically binding to a c-Met polypeptide comprising a mutation in exons 2, 3, 4, 7, 9, 13, 14, 15, 17, 19, 20 and/or 21 that encode the c-Met polypeptide.

A preferred form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A preferred form for a solid-state substrate is a microtiter dish, such as a standard 96-well type. In preferred embodiments, a multiwell glass slide can be employed that normally contain one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of the polynucleotides or polypeptides disclosed herein.

Although preferred, it is not required that a given array be a single unit or structure. The set of compounds may be distributed over any number of solid supports. For example, at one extreme, each compound may be immobilized in a separate reaction tube or container, or on separate beads or microparticles. Different modes of the disclosed method can be performed with different components (for example, different compounds specific for different proteins) immobilized on a solid support.

Some solid supports can have capture compounds, such as antibodies, attached to a solid-state substrate. Such capture compounds can be specific for calcifying nano-particles or a protein on calcifying nano-particles. Captured calcifying nano-particles or proteins can then be detected by binding of a second, detection compound, such as an antibody. The detection compound can be specific for the same or a different protein on the calcifying nano-particle.

Methods for immobilizing antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy] succinimide ester (GMBS). These and other attachment agents, as well as methods for their use in attachment, are described in Protein immobilization: fundamentals and applications, Richard F. Taylor, ed. (M. Dekker, New York, 1991); Johnstone and Thorpe, Immunochemistry In Practice (Blackwell Scientific Publications, Oxford, England, 1987) pages 209-216 and 241-242, and Immobilized Affinity Ligands; Craig T. Hermanson et al., eds. (Academic Press, New York, 1992) which are incorporated by reference in their entirety for methods of attaching antibodies to a solid-state substrate. Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A preferred method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS³) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Optionally, at least one address on the solid support is the sequences or part of the sequences set forth in any of the nucleic acid sequences disclosed herein. Also disclosed are solid supports where at least one address is the sequences or portion of sequences set forth in any of the peptide sequences disclosed herein. Solid supports can also contain at least one address is a variant of the sequences or part of the sequences set forth in any of the nucleic acid sequences disclosed herein. Solid supports can also contain at least one address is a variant of the sequences or portion of sequences set forth in any of the peptide sequences disclosed herein.

Also disclosed are antigen microarrays for multiplex characterization of antibody responses. For example, disclosed are antigen arrays and miniaturized antigen arrays to perform large-scale multiplex characterization of antibody responses directed against the polypeptides, polynucleotides and antibodies described herein, using submicroliter quantities of biological samples as described in Robinson et al., Autoantigen microarrays for multiplex characterization of autoantibody responses, Nat. Med., 8(3):295-301 (2002), which in herein incorporated by reference in its entirety for its teaching of contructing and using antigen arrays to perform large-scale multiplex characterization of antibody responses directed against structurally diverse antigens, using submicroliter quantities of biological samples.

Protein variants and derivatives are well understood to those of skill in the art and can involve amino acid sequence modifications. For example, amino acid sequence modifications typically fall into one or more of three classes: substitutional, insertional or deletional variants. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

Also disclosed are expression vectors comprising the polynucleotides described elsewhere herein. For example, disclosed are expression vectors comprising the polynucleotides described elsewhere herein, operably linked to a control element. Also disclosed herein are host cells transformed or transfected with an expression vector comprising the polynucleotides described elsewhere herein. Also disclosed are host cells comprising the expression vectors described herein. For example, disclosed is a host cell comprising an expression vector comprising the polynucleotides described elsewhere herein, operably linked to a control element. Host cells can be eukayotic or prokaryotic cells.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). For example, disclosed herein are expression vectors comprising an isolated polynucleotide comprising a sequence of SEQ ID NOs: 1-21, 23, 25, 27, 29, 31, 33, 35, 37, 39, 41, 43, 45, 47 or 49 operably linked to a control element.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

Preferred promoters controlling transcription from vectors in mammalian host cells may be obtained from various sources, for example, the genomes of viruses such as polyoma, Simian Virus 40 (SV40), adenovirus, retroviruses, hepatitis-B virus and most preferably cytomegalovirus, or from heterologous mammalian promoters (e.g. beta actin promoter). The early and late promoters of the SV40 virus are conveniently obtained as an SV40 restriction fragment, which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Additionally, promoters from the host cell or related species can also be used.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product is used to determine if the gene has been delivered to the cell and once delivered is being expressed. Preferred marker genes are the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hygromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as an isolated polynucleotide capable of encoding one or more polypeptides selected from the group consisting of SEQ ID NOs: 3-26 into the cell without degradation and include a promoter yielding expression of the gene in the cells into which it is delivered. In some embodiments the isolated polynucleotides disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The disclosed vectors thus can provide DNA molecules that are capable of integration into a mammalian chromosome without substantial toxicity.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors.

In addition, the disclosed polynucleotides can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood, to a target organ, or inhaled into the respiratory tract to target cells of the respiratory tract. For example, a composition comprising a polynucleotide described herein and a cationic liposome can be administered to a subjects lung cells. Regarding liposomes, see, e.g., Brigham et al. Am. J. Resp. Cell. Mol. Biol. 1:95-100 (1989); Feigner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described herein, delivery of the compositions to cells can be via a variety of mechanisms. As one example, delivery can be via a liposome, using commercially available liposome preparations such as LIPOFECTIN™, LIPOFECTAMINE™ (GIBCO-BRL, Gaithersburg, Md.), SUPERFECT™ (Qiagen, Hilden, Germany) and TRANSFECTAM™ (Promega Biotec, Madison, Wis.), as well as other liposomes developed according to procedures standard in the art. In addition, the disclosed nucleic acid or vector can be delivered in vivo by electroporation, the technology for which is available from Genetronics (San Diego, Calif.) as well as by means of a SONOPORATION™ machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

As described herein, the compositions can be administered in a pharmaceutically acceptable carrier and can be delivered to the subject's cells in vivo or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The nucleic acids, such as, the polynucleotides described herein, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 3rd Edition (Cold Spring Harbor Laboratory Press, Cold Spring Harbor, N.Y., 2001) Chapters 5, 6) to purely synthetic methods, for example, by the cyanoethyl phosphoramidite method using a Milligen or Beckman System 1Plus DNA synthesizer. Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

The invention also provides polypeptides related to c-Met. As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Thus, a “c-Met polypeptide” or “c-Met protein,” refers generally to a polypeptide sequence of the present invention that is present in samples isolated from a substantial proportion of subjects with a cancer, for example preferably greater than about 20%, more preferably greater than about 30%, and most preferably greater than about 50% or more of subjects tested as determined using a representative assay provided herein. A polypeptide sequence of the invention, based upon its expression in a cancer sample isolated from individuals with a cancer, has particular utility both as a diagnostic marker as well as a therapeutic target, as further described below.

For example, disclosed herein are polypeptides comprising an amino acid sequence encoded by the polynucleotides described elsewhere herein. For example, disclosed is an isolated polypeptide comprising an amino acid sequence provided in SEQ ID NOS: 3-26. Also disclosed are isolated polypeptides capable of encoding one or more polypeptides selected from the group consisting of SEQ ID NOs: 3-26, or a complement thereof.

The polypeptides of the present invention are sometimes herein referred to as c-Met proteins or c-Met polypeptides, as an indication that their identification has been based at least in part upon their expression in cancer samples isolated from tissues of a subject with lung cancer, head and neck cancer, or melanoma. The peptides described herein are identified from tissues for a subject with either lung cancer, head and neck cancer, or melanoma. Accordingly, such a peptide may not be present in adjacent normal tissue.

Additionally, polypeptides described herein may be identified by their different reactivity with sera from subjects with cancer as compared to sera from unaffected individuals. For example, polypeptides described herein may be identified by their reactivity with sera from subjects with a cancer as compared to their lack of reactivity to sera from unaffected individuals. Additionally, polypeptides described herein may be identified by their reactivity with sera from subjects with cancer as compared to their higher reactivity to sera from unaffected individuals. Additionally, polypeptides described herein may be identified by their reactivity with sera from subjects with a cancer as compared to their lower reactivity to sera from unaffected individuals.

Also disclosed herein are antigen binding agents capable of specifically binding to a c-Met polypeptide comprising a mutation at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed is an antigen binding agent capable of specifically binding to a c-Met polypeptide comprising a mutation in exons 2, 3, 4, 7, 9, 13, 14, 15, 17, 19, 20 and/or 21 that encode the c-Met polypeptide.

Also disclosed are isolated polypeptides comprising the sequence provided in SEQ ID NOS: 3-26, with substituted, inserted or deletional variations.

Insertions include amino or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion.

Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | K |
| glycine | Gly | G |
| histidine | His | H |
| isolelucine | Ile | I |
| leucine | Leu | L |
| lysine | Lys | K |
| phenylalanine | Phe | F |
| proline | Pro | P |
| serine | Ser | S |
| threonine | Thr | T |
| tyrosine | Tyr | Y |
| tryptophan | Trp | W |
| valine | Val | V |

Substantial changes in function or immunological identity are made by selecting substitutions that are less conservative than those in Tables 1 and 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation or glycosylation.

TABLE 2
Amino Acid Substitutions
Original Residue Exemplary Conservative
Substitutions, others are known in the art.

ala; ser
arg; lys; gln
asn; gln; his
asp; glu
cys; ser
gln; asn; lys
glu; asp
gly; pro
his; asn; gln
ile; leu; val
Leu; ile; val
lys; arg; gln;
Met; leu; ile
phe; met; leu; tyr
ser; thr
thr; ser
trp; tyr
tyr; trp; phe
val; ile; leu

For example, the replacement of one amino acid residue with another that is biologically and chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or β-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

It is understood that the description of conservative mutations and homology can be combined together in any combination, such as embodiments that have at least 70% homology to a particular sequence wherein the variants are conservative mutations.

It is understood that there are numerous amino acid and peptide analogs which can be incorporated into the disclosed compositions. For example, there are numerous D amino acids or amino acids which have a different functional substituent then the amino acids shown in Tables 1 and 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

As this specification discusses various polypeptides and polypeptide sequences it is understood that the nucleic acids that can encode those polypeptide sequences are also disclosed. This would include all degenerate sequences related to a specific polypeptide sequence, i.e. all nucleic acids having a sequence that encodes one particular polypeptide sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed polypeptide sequences.

Also disclosed herein are isolated antibodies, antibody fragments and antigen-binding fragments thereof, that specifically bind to a polypeptide sequence selected from the group consisting of SEQ ID NOs: 3-26. Optionally, the isolated antibodies, antibody fragments, or antigen-binding fragment thereof can be neutralizing antibodies. The antibodies, antibody fragments and antigen-binding fragments thereof disclosed herein can be identified using the methods disclosed herein. For example, antibodies that bind to the polypeptides of the invention can be isolated using the antigen microarray described above.

The term “antibodies” is used herein in a broad sense and includes both polyclonal and monoclonal antibodies. In addition to intact immunoglobulin molecules, also disclosed are antibody fragments or polymers of those immunoglobulin molecules, and human or humanized versions of immunoglobulin molecules or fragments thereof, as long as they are chosen for their ability to interact with the polypeptides disclosed herein.

“Antibody fragments” are portions of a complete antibody. A complete antibody refers to an antibody having two complete light chains and two complete heavy chains. An antibody fragment lacks all or a portion of one or more of the chains. Examples of antibody fragments include, but are not limited to, half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups or coupling of biomolecules to the antibody fragment. A preferred half antibody fragment is a F(ab). The hinge region of an antibody or antibody fragment is the region where the light chain ends and the heavy chain goes on.

Antibody fragments for use in antibody conjugates can bind antigens. Preferably, the antibody fragment is specific for an antigen. An antibody or antibody fragment is specific for an antigen if it binds with significantly greater affinity to one epitope than to other epitopes. The antigen can be any molecule, compound, composition, or portion thereof to which an antibody fragment can bind. An analyte can be any molecule, compound or composition of interest. For example, the antigen can be a polynucleotide of the invention.

The antibodies or antibody fragments can be tested for their desired activity using the in vitro assays described herein, or by analogous methods, after which their in vivo therapeutic or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. Also disclosed are “chimeric” antibodies in which a portion of the heavy or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, such as an Fv, Fab, Fab', or other antigen-binding portion of an antibody, can be accomplished using routine techniques known in the art. For instance, digestion can be performed using papain. Examples of papain digestion are described in WO 94/29348 published Dec. 22, 1994 and U.S. Pat. No. 4,342,566 which is hereby incorporated by reference in its entirety for its teaching of papain digestion of antibodies to prepare monovaltent antibodies. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Optionally, the disclosed human antibodies can be made from memory B cells using a method for Epstein-Barr virus transformation of human B cells. (See, e.g., Triaggiai et al., An efficient method to make human monoclonal antibodies from memory B cells: potent neutralization of SARS coronavirus, Nat. Med. 2004 Aug.; 10(8):871-5. (2004)), which is herein incorporated by reference in its entirety for its teaching of a method to make human monoclonal antibodies from memory B cells). In short, memory B cells from a subject who has survived a natural infection are isolated and immortalized with EBV in the presence of irradiated mononuclear cells and a CpG oligonucleotide that acts as a polyclonal activator of memory B cells. The memory B cells are cultured and analyzed for the presence of specific antibodies. EBV-B cells from the culture producing the antibodies of the desired specificity are then cloned by limiting dilution in the presence of irradiated mononuclear cells, with the addition of CpG 2006 to increase cloning efficiency, and cultured. After culture of the EBV-B cells, monoclonal antibodies can be isolated. Such a method offers (1) antibodies that are produced by immortalization of memory B lymphocytes which are stable over a lifetime and can easily be isolated from peripheral blood and (2) the antibodies isolated from a primed natural host who has survived a natural infection, thus eliminating the need for immunization of experimental animals, which may show different susceptibility and, therefore, different immune responses.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab', or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

The antibodies disclosed herein can also be administered to a subject. Nucleic acid approaches for antibody delivery also exist. The broadly neutralizing antibodies to the polypeptides disclosed herein and antibody fragments can also be administered to subjects or subjects as a nucleic acid preparation (e.g., DNA or RNA) that encodes the antibody or antibody fragment, such that the subject's own cells take up the nucleic acid and produce and secrete the encoded antibody or antibody fragment.

The compositions disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the inflammatory disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated herein by reference in its entirety for its teaching of an approach for parenteral administration.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)), all of which are herein incorporated by reference in their entirety for their teaching of the same. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carriers include, but are not limited to, sterile water, saline, Ringer's solution, dextrose solution, and buffered solutions at physiological pH. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the polynucleotide, polypeptide, antibody, T-cell, TCR, or APC compositions disclosed herein. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Also disclosed are illustrative immunogenic compositions, e.g., vaccine compositions, that comprise DNA encoding one or more of the polypeptides as described above, such that the polypeptide is generated in situ. Numerous gene delivery techniques are well known in the art, such as those described by Rolland, Crit. Rev. Therap. Drug Carrier Systems 15:143-198, 1998, and references cited therein, all of which are herein incorporated by reference in their entirety for their teaching of gene delivery techniques. Appropriate polynucleotide expression systems contain the necessary regulatory DNA regulatory sequences for expression in a subject (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Additionally, the pharmaceutical compositions described herein can comprise one or more immunostimulants in addition to the polynucleotide, polypeptide, antibody, T-cell, TCR, or APC compositions of this invention. An immunostimulant refers to essentially any substance that enhances or potentiates an immune response (antibody or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Rahway, N.J.); AS-2 (GlaxoSmithKline, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

The adjuvant composition can be a composition that induces an anti-inflammatory immune response (antibody or cell-mediated). Accordingly, high levels of anti-inflammatory cytokines (anti-inflammatory cytokines may include, but are not limited to, interleukin 4 (IL-4), interleukin 5 (IL-5), interleukin 10 (IL-10), and transforming growth factor beta (TGFβ). Optionally, an anti-inflammatory response would be mediated by CD4+ T helper cells. Bacterial flagellin has been shown to have adjuvant activity (McSorley et al., J. Immunol. 169:3914-19, 2002). Also disclosed are polypeptide sequences that encode flagellin proteins that can be used in adjuvant compositions.

Optionally, the adjuvants used in conjunction with the compositions of the present invention increase lipopolysaccharide (LPS) responsiveness. Illustrative adjuvants include but are not limited to, monophosphoryl lipid A (MPL), aminoalkyl glucosaminide 4-phosphates (AGPs), including, but not limited to RC-512, RC-522, RC-527, RC-529, RC-544, and RC-560 (Corixa, Hamilton, Mont.) and other AGPs such as those described in pending U.S. patent application Ser. Nos. 08/853,826 and 09/074,720, the disclosures of which are incorporated herein by reference in their entireties.

In addition, the adjuvant composition can be one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a subject will support an immune response that includes Th1- and Th2-type responses. Optionally, the level of Th1-type cytokines will increase to a greater extent than the level of Th2-type cytokines. The levels of these cytokines may be readily assessed using standard assays. For a review of the families of cytokines, see Mosmann and Coffman, Atm. Rev. Immunol. 7:145-173, 1989, which is hereby incorporated by reference for its teaching of families of cytokines. The level of Th2-type cytokines can increase to a greater extent than the level of Th1-type cytokines.

Certain adjuvants for eliciting a predominantly Th1-type response include, for example, a combination of monophosphoryl lipid A, preferably 3-de-O-acylated monophosphoryl lipid A, together with an aluminum salt adjuvants are available from Corixa Corporation (Seattle, Wash.; see, for example, U.S. Pat. Nos. 4,436,727; 4,877,611; 4,866,034 and 4,912,094, which are hereby incorporated by reference for their teaching of the same). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other formulations can include more than one saponin in the adjuvant combinations of the present invention, for example combinations of at least two of the following group comprising QS21, QS7, Quil A, β-escin, or digitonin.

Saponin formulations can also be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins can also be formulated in the presence of cholesterol to form particulate structures such as liposomes or immune-stimulating complexes (ISCOMs). Furthermore, the saponins may be formulated together with a polyoxyethylene ether or ester, in either a non-particulate solution or suspension, or in a particulate structure such as a paucilamelar liposome or ISCOM. The saponins can also be formulated with excipients such as CARBOPOL™ (Noveon, Cleveland, Ohio) to increase viscosity, or may be formulated in a dry powder form with a powder excipient such as lactose.

Optionally, the adjuvant system includes the combination of a monophosphoryl lipid A and a saponin derivative, such as the combination of QS21 and 3D-MPL. adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Optionally the formulation additionally comprises an oil in water emulsion and tocopherol.

Additional illustrative adjuvants for use in the pharmaceutical compositions of the invention include Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from GlaxoSmithKline, Philadelphia, Pa.), Detox (Enhanzyn™) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the subject, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 mg/kg to up to 100 mg/kg of body weight or more per day, depending on the factors mentioned above.

Following administration of a disclosed composition, such as an antibody, for treating, inhibiting, or preventing an immune-mediated inflammatory disease, the efficacy of the therapeutic antibody can be assessed in various ways well known to the skilled practitioner.

The compositions that inhibit inflammatory interactions disclosed herein may be administered prophylactically to subjects or subjects who are at risk for cancer.

The disclosed compositions and methods can also be used for example as tools to isolate and test new drug candidates for a variety of cancers.

In addition, the compositions described herein may be used as markers for presence or progression of cancers. The methods and assays described elsewhere herein may be performed over time, and the change in the level of reactive polypeptide(s) or polynucleotide(s) evaluated. For example, the assays may be performed every 24-72 hours for a period of 6 months to 1 year, and thereafter performed as needed. For example, immunoreactivity to a given polypeptide in individuals with cancer can correlate with or predict the development of complications, more severe activity of disease.

As noted herein, to improve sensitivity, multiple mutations may be assayed within a given sample. Binding agents specific for different proteins, antibodies, nucleic acids thereto provided herein may be combined within a single assay. Further, multiple primers or probes may be used concurrently. The selection of c-Met proteins may be based on routine experiments to determine combinations that results in optimal sensitivity. In addition, or alternatively, assays for mutated c-Met, antibodies, or nucleic acids specific thereto, provided herein may be combined with assays for other known cancer markers or other genetic markers in subjects with cancer. To assist with such assays, specific biomarkers can assist in the specificity of such tests. As such, disclosed herein is a cancer biomarker, wherein the biomarker comprises c-met comprising a mutation that results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed is a cancer biomarker, wherein the biomarker comprises c-met comprising a mutation in exons 2, 3, 4, 7, 9, 13, 14, 15, 17, 19, 20 and/or 21, and/or their flanking introns, wherein the mutation affects exon splicing and/or protein structure.

The biomarkers described herein can be in any form that provides information regarding presence or absence of a mutation of the invention. For example, the disclosed biomarkers can be, but is not limited to a nucleic acid molecule, a polypeptide, or an antibody.

Also disclosed are cancer imaging agents, wherein the agent specifically binds c-Met comprising a mutation, wherein the agent binds a c-Met polypeptide comprising a mutation at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I of the protein, or wherein the agent binds a c-Met encoding nucleic acid comprising a mutation at a nucleic acid position corresponding to a change in amino acid at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Further disclosed are cancer imaging agents, wherein the agent specifically binds c-Met polypeptide comprising a deletion of at least a portion of exons 2, 3, 4, 7, 9, 13, 14, 15, 17, 19, 20 and/or 21, or wherein the agent specifically binds c-Met encoding nucleic acid that comprises a mutation in a sequence that encodes exons 2, 3, 4, 7, 9, 13, 14, 15, 17, 19, 20 and/or 21.

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once a cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

Disclosed herein are transgenic animals comprising mutations in a nucleotide sequence capable of encoding a c-Met protein. Transgenic animals include, but are not limited to zebrafish and nematodes. It is also understood that the animal can comprise any mammal. For example, the animal can be a mouse, vole, rat, guinea pig, cat, dog, cow, sheep pig, monkey, or human. For example, disclosed are transgenic animal comprising one or more of the disclosed c-met mutations including, but not limited to c-met encoding nucleic acids comprising a mutation at a nucleic acid position corresponding to a change in amino acid at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed are transgenic animals comprising one or more of the disclosed c-met mutations including, but not limited to c-met encoding nucleic acids that comprises a mutation in a sequence that encodes exons 2, 3, 4, 7, 9, 13, 14, 15, 17, 19, 20 and/or 21 of the c-met.

Transgenic animals as described above can be generated as discussed in the Examples below.

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein. Also disclosed are computer readable mediums comprising the sequences and information regarding the sequences set forth herein.

Also disclosed herein is a computer-readable medium comprising human c-Met amino acid polypeptide sequence comprising a mutation at position L211W, T230M, S244P, L229F, F253S, S323G, A347T, E355K, R359Q, M362T, M431V, N454I, S470L, I852F, N948S, S1058P, R988C, T1010I, Q1029E, S1167N, T1275I, P1300S, P1301S, and/or V1333I, and/or nucleic acid sequence encoding a human c-Met polypeptide comprising a mutation at a nucleic acid position corresponding to a change in amino acid at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Further disclosed is a computer-readable medium comprising human c-Met amino acid polypeptide sequence comprising a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, and/or human c-Met encoding nucleic acid that a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns.

The computer-readable mediums disclosed herein can comprise a storage medium for sequence information for one or more subjects. For example, the information can be a personalized genomic profile for a subject known or suspected to have a cancer, wherein the genomic profile comprises sequence information for c-met comprising one or more of the mutations disclosed herein.

### Methods

The disclosed compositions, including the c-met mutations disclosed herein can be used in a variety of different methods, for example in prognostic, predictive, diagnostic, and therapeutic methods and as a variety of different compositions. For example, disclosed herein are prognostic methods comprising determining whether a cancer sample from a subject comprises a mutation in a nucleic acid sequence encoding human c-Met. Cancers or cancer tissues that can be used in the disclosed methods include, but are not limited to, lymphoma (Hodgkins and non-Hodgkins) B-cell lymphoma, T-cell lymphoma, leukemia such as myeloid leukemia and other types of leukemia, mycosis fungoide, carcinoma, adenocarcinoma, sarcoma, glioma, astrocytoma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, lung cancer, head and neck carcinoma, hematopoietic cancer, testicular cancer, colo-rectal cancer, prostatic cancer, and pancreatic cancer. Specific lung cancers that can be used in the disclosed methods include, but are not limited to Non-Small Cell Lung Cancers (NSCLC) and/or Squamous Cell Carcinomas (SCC).

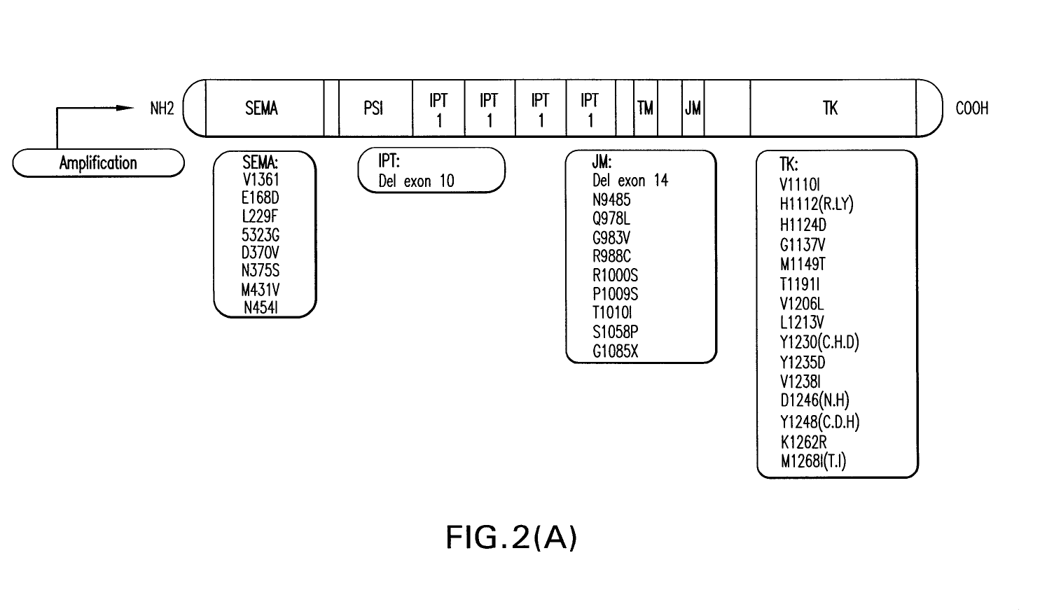

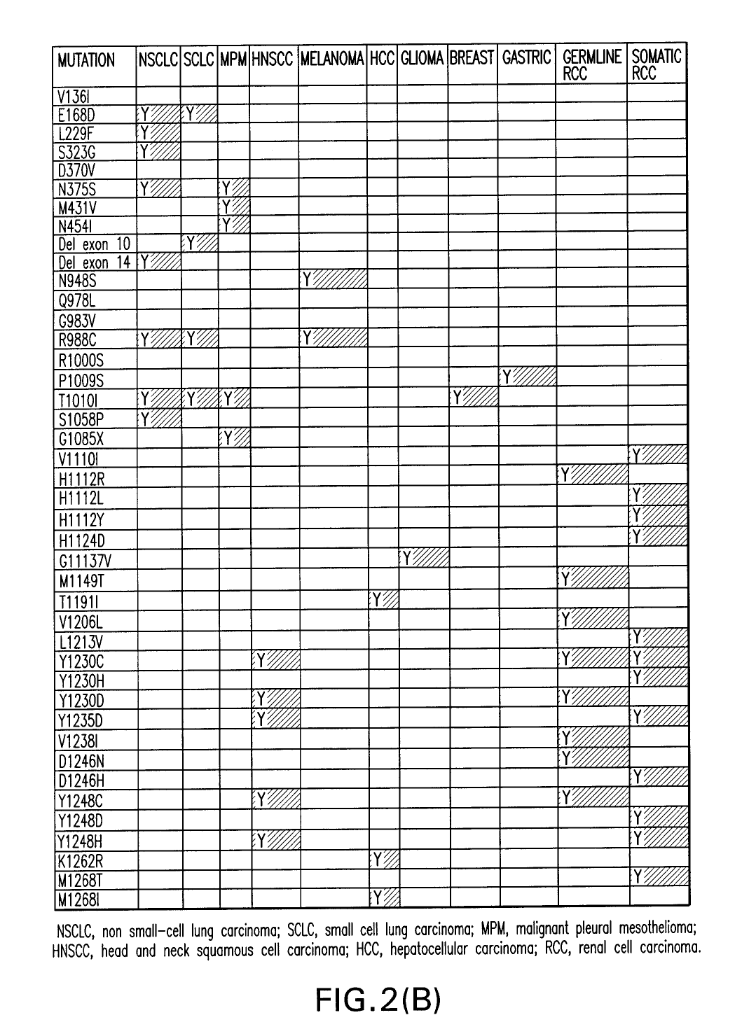

For example, disclosed herein are prognostic methods comprising determining whether a cancer sample from a subject comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed are prognostic methods comprising determining whether a cancer sample from a subject comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure. For example, the mutation can alter the protein structure as indicated in FIG. 2b.

Also disclosed herein, are methods of detecting cancer in a sample comprising determining whether the sample comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed are methods of detecting cancer in a sample comprising determining whether the sample comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

Also disclosed are methods for distinguishing between non-cancerous and cancerous tissue. For example, disclosed are methods for distinguishing between non-cancerous and cancerous tissue, said method comprising determining whether a sample comprising the tissue comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I, wherein detection of the mutation in the sample is indicative of presence of cancerous tissue. Also disclosed is a method for distinguishing between non-cancerous and cancerous tissue, said method comprising determining whether a sample comprising the tissue comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure, wherein detection of the mutation in the sample is indicative of presence of cancerous tissue.

As used herein, “cancerous tissue” is meant to mean a tissue that comprises neoplastic cells, exhibits an abnormal growth of cells and/or hyperproliferative cells. As used herein, the term “neoplastic” means an abnormal growth of a cell or tissue (e.g., a tumor) which may be benign or cancerous. As used herein, “abnormal growth of cells” and/or “hyperproliferative cells” are meant to refer to cell growth independent of normal regulatory mechanisms (e.g., loss of contact inhibition), including the abnormal growth of benign and malignant cells or other neoplastic diseases. As used herein, the term “tumor” includes neoplasms that are identifiable through clinical screening or diagnostic procedures including, but not limited to, palpation, biopsy, cell proliferation index, endoscopy, mammography, digital mammography, ultrasonography, computed tomography (CT), magnetic resonance imaging (MRI), positron emission tomography (PET), radiography, radionuclide evaluation, CT- or MRI-guided aspiration cytology, and imaging-guided needle biopsy, among others. Such diagnostic techniques are well known to those skilled in the art and are described in Holland, et al., Cancer Medicine, 4th Ed., Vol. One, Williams & Wilkins, Baltimore, Md. (1997).

TABLE 3

| Exon | Nucleotide | Amino acid |
|---|---|---|
| Exon 2 | G504T | E168D |
| Exon 2 | C534T | S178S |
| Exon 2 | T730C | S244P |
| Exon 2 | T758C | F253S |
| Exon 2 | G1076A | R359Q |
| Exon 2 | A1124G | N375S |
| Exon 4 | C1409T | S470L |
| Exon 7 | A1944G | Q648Q |
| Exon 10 | Alternatively spliced | |
| Intron 13 | IVS13-(52-53)insCT | |
| Exon 14 | C2962T | R988C |
| Exon 14 | C3029T | T1010I |
| Exon 17 | G3500A | S1167N |
| Exon 20 | C3912T | D1304D |
| Exon 21 | G4071A | A1357A |
| Exon 21 | G4146A | P1382P |

TABLE 4

| Exon | Nucleotide | Amino acid |
|---|---|---|
| Exon 2 | C534T | S178S |
| Exon 2 | G1039A | A347T |
| Exon 2 | G1063A | E355K |
| Exon 2 | T1085C | M362T |
| Exon 2 | A1124G | N375S |
| Exon 2 | C1131T | I377I |
| Exon 14 | C2962T | R988C |
| Exon 15 | C3085G | Q1029E |
| Exon 17 | A3552C | R1184R |
| Exon 20 | C3912T | D1304D |
| Exon 21 | G4071A | A1357A |
| Exon 21 | G4146A | P1382P |

TABLE 5

| Exon | Nucleotide | Amino acid |
|---|---|---|
| Exon 2 | G144A | A48A |
| Exon 2 | G504T | E168D |
| Exon 2 | C534T | S178S |
| Exon 2 | C689T | T230M |
| Exon 7 | A1944G | Q648Q |
| Exon 9 | T2118C | I706I |
| Exon 14 | C2962T | R988C |
| Exon 14 | C3029T | T1010I |
| Exon 19 | A3750G | K1250K |
| Exon 19 | C3824T | T1275I |
| Exon 20 | C3898T | P1300S |
| Exon 20 | C3901T | P1301S |
| Exon 20 | C3912T | D1304D |
| Exon 21 | G3997A | V1333I |
| Exon 21 | G4071A | A1357A |
| Exon 21 | G4146A | P1382P |

TABLE 6

| Exon | Amino acid |
|---|---|
| 13 | N948S |
| 14 | R988C |

Also described are methods of identifying a mutation in c-met in a cancer. For example, described are methods of identifying a mutation in c-met in a cancer, said method comprising contacting a cancer sample with an agent capable of detecting a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also described are methods of identifying a mutation in c-met in a cancer, said method comprising contacting a cancer sample with an agent capable of detecting a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

Due to the vast information supporting the role of c-Met and HGF in the pathogenesis of human cancers along with successes of other RTK inhibitors, a number of approaches have been attempted to inhibit HGF- or c-Met-dependent signaling. These approaches include: (1) c-Met biologic inhibitors (ribozymes, dominant-negative receptors, decoy receptors, peptides); (2) HGF kringle variant antagonists; (3) HGF antagonist antibodies; (4) c-Met antagonist antibodies; and (5) small-molecule c-Met inhibitors. To date, several possible c-Met inhibitors have been developed with the intent on either silencing, or decreasing c-Met expression or decreasing c-Met activity. For example, Compound X (a third generation c-met inhibitor), PHA665752 (Pfizer, Inc.), SU11274 (Sugen, Inc.), SU11271 (Sugen, Inc.), SU11606 (Sugen, Inc.), ARQ197 (ArQuleArqule, Inc.), MP470 (Supergen, Inc.), Kirin, XL-880 (Exelixis, Inc.), XL184 (Exelixis, Inc.) Geldanamycins, SGX523 (SGX, Inc.), MGCD265 (MethylGene, Inc.), HPK-56 (Supergen, Inc.), AMG102 (Amgen, Inc.), MetMAb (Genentech, Inc.), ANG-797 (Angion Biomedica Corp.), CGEN-241 (Compugen LTD.), Metro-F-1 (Dompe S.p.A.), ABT-869 (Abbott Laboratories) and K252a are all c-Met inhibitors currently being produced.

While some candidates appear to be successful in inhibiting wild-type c-Met, that is to say c-Met without any mutations, it is unknown how the inhibitors will affect the expression and/or the activity of a mutated version of c-Met. Disclosed herein are methods of identifying a cancer that is susceptible to treatment with a c-Met inhibitor and/or predicting the likelihood that a cancer will respond to treatment with a c-Met inhibitor and/or predicting/identifying which patients diagnosed with cancer to treatment with a c-Met inhibitor. For example, disclosed are methods of identifying a cancer that is susceptible to treatment with a c-Met inhibitor, said method comprising determining whether a cancer sample from a subject comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Also disclosed are methods of identifying a cancer that is susceptible to treatment with a c-Met inhibitor, said method comprising determining whether a cancer sample from a subject comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure. Susceptibility can either mean that the cancer sample comprising a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I, is less responsive to a c-Met inhibitor or more responsive to a c-Met inhibitor. For example, a cancer sample comprising a mutation in a nucleic acid sequence encoding human c-Met, wherein the mutation results in an amino acid change at position N375 or R988C is more responsive to a c-Met inhibitor.

Also disclosed are methods of determining responsiveness of a cancer in a subject to treatment with a c-Met inhibitor. For example, disclosed are methods of determining responsiveness of a cancer in a subject to treatment with a c-Met inhibitor, said method comprising determining whether a cancer sample from a subject who has been treated with the c-Met inhibitor comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, T1010I, Q1029E, S1167N, T1275I, P1300S, P1301S, and/or V1333I, wherein absence of the mutated nucleic acid sequence is indicative that the cancer is responsive to treatment with the c-Met inhibitor.

Also disclosed are methods of determining responsiveness of a cancer in a subject to treatment with a c-Met inhibitor, said method comprising determining whether a cancer sample from a subject who has been treated with the c-Met inhibitor comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure, wherein absence of the mutated nucleic acid sequence is indicative that the cancer is responsive to treatment with the c-met inhibitor.

Also disclosed are methods for monitoring minimal residual cancer in a subject treated for cancer with a c-Met inhibitor. For example, disclosed are methods for monitoring minimal residual cancer in a subject treated for cancer with a c-Met inhibitor, said methods comprising determining whether a sample from a subject who is treated with the c-Met inhibitor comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I, wherein detection of said mutation is indicative of presence of minimal residual cancer. Also, disclosed are methods for monitoring minimal residual cancer in a subject treated for cancer with a c-Met inhibitor, said method comprising determining whether a sample from a subject who has been treated with the c-Met inhibitor comprises a mutation in a nucleic acid sequence encoding human c-Met, wherein the sequence is mutated in one or more of exons 2, 3, 4, 7, 9, 14, 15, 17, 19, 20 and/or 21, and/or their flanking introns, wherein the mutation affects exon splicing and/or protein structure, wherein detection of said mutation is indicative of presence of minimal residual cancer.

Also disclosed are methods for amplification of a nucleic acid encoding human c-Met. For example, disclosed are methods for amplification of a nucleic acid encoding human c-Met, wherein the nucleic acid comprises a mutation that results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I relative to wild type c-Met, said method comprising amplifying a sample suspected or known to comprise the a nucleic acid comprising the sequence of any of the primers/probes listed in Tables 7-10. Also disclosed are methods for amplification of a nucleic acid encoding human c-Met, wherein the nucleic acid comprises a mutation in exons 2, 3, 4, 7, 9, 13, 14, 15, 17, 19, 20 and/or 21, and/or their flanking introns, wherein the mutation affects exon splicing and/or protein structure, said method comprising amplifying a sample suspected or known to comprise the nucleic acid with a nucleic acid comprising the sequence of any of the primers/probes listed in Tables 7-10.

TABLE 7

| No. | Exon | Sequence |
|---|---|---|
| 1 | Exon 2 | GACAACTGAACTGCTCTCGCCTTGAACC (SEQ ID |
| | | NO. 27) |
| | | GGTGTTCCAGCACAGGCCCAGTC (SEQ ID NO. 28) |
| 2 | Exon 2 | GACTGGGCCTGTGCTGGAACACC (SEQ ID NO. 29) |
| | | GATGAAAGGACTTTGGCTCCCAGGGC (SEQ ID NO. |
| | | 30) |
| 3 | Exon 2 | GCCCTGGGAGCCAAAGTCCTTTCATC (SEQ ID NO. |
| | | 31) |
| | | CACTCCAGAGGCATTTCCATGTAGGAATGC (SEQ |
| | | ID NO. 32) |
| 4 | Exon 2 | GCATTCCTACATGGAAATGCCTCTGGAGTG (SEQ |
| | | ID NO. 33) |
| | | CCTATTAAAGCAGTGCTCATGATTGGGTCCG (SEQ |
| | | ID NO. 34) |
| 5 | Exon 2 | CATGTCTTTCCCCACAATCATACTGCTGA (SEQ ID |
| | | NO. 35) |
| | | CAGGTTCTGTTCCATAAACTCTGGATTGC (SEQ ID |
| | | NO. 36) |
| 6 | Exon 3 | TGATTATCCTTGCCATTATCCTCCAGGCTCTG |
| | | (SEQ ID NO. 37) |
| | | ACCAGATAGAACAGACACAGCTACTCTCAG (SEQ |
| | | ID NO. 38) |
| 7 | Exon 4 | GGTCTTCAGTTCAAACACCCACAAGC (SEQ ID NO. |
| | | 39) |
| | | GGCCAAGTGACACTGGTTGTAAATATGC (SEQ ID |
| | | NO. 40) |
| 8 | Exon 15 | GCTCTTCCTGTTTCAGTCCCCATTAAATGAGG |
| | | (SEQ ID NO. 41) |
| | | CACCATTGTCTAAGTTCCTAATCTGCAAAGGCC |
| | | (SEQ ID NO. 42) |
| 9 | Exon 16 | GTTACGCAGTGCTAACCAAGTTCTTTC (SEQ ID |
| | | NO. 43) |
| | | CCCCCTTACTAATGAGGGCTCTGAGGG (SEQ ID |
| | | NO. 44) |
| 10 | Exon 17 | CCCTCAGGACAAGATGCTAACTGTGTGG (SEQ ID |
| | | NO. 45) |
| | | GGGATGGCTGGCTTACAGCTAGTTTGC (SEQ ID |
| | | NO. 46) |
| 11 | Exon 18 | ACTCCTGGCCTCAAGCCATCCTCTC (SEQ ID NO. |
| | | 47) |
| | | GGATTGTGGCACAGAGATTCTGATACTTAC (SEQ |
| | | ID NO. 48) |
| 12 | Exon 20 | GCCAAGTTTAGTTACCAAGACCTACTGATTTC |
| | | (SEQ ID NO. 49) |
| | | GAGACCCTTTGAAGGCAGGCATTTC (SEQ ID NO. |
| | | 50) |
| 13 | Exon19 | CTCAGCCTGTTGAATTGGCAATGTCAATGTC (SEQ |
| | | ID NO. 51) |
| | | CAGAGATAACCAATACATTACCACATCTGACTTG |
| | | G (SEQ ID NO. 52) |

TABLE 8

| | | |
|---|---|---|
| Primer | | |
| No. | Exon | Sequence |

| | | |
|---|---|---|
| 1 | Exon 2 | Fwd: GACAACTGAACTGCTCTCGCCTTGAACC (SEQ ID NO. |
| | | 53) |
| | | Rev: GAGAATATGCAGTGAACCTCCGACTGTATG (SEQ ID |
| | | NO. 54) |
| 2 | Exon 2 | Fwd: GACTGGGCCTGTGCTGGAACAGC (SEQ ID NO. 55) |
| | | Rev: GATGAAAGGACTTTGGCTCCCAGGGC (SEQ ID NO. 56) |
| 3 | Exon 2 | Fwd: GCCCTGGGAGCCAAAGTCCTTTCATC (SEQ ID NO. 57) |
| | | Rev: CACTCCAGAGGCATTTCCATGTAGGAATGC (SEQ ID |
| | | NO. 58) |
| 4 | Exon 2 | Fwd: CATGTCTTTCCCCACAATCATACTGCTGA (SEQ ID NO. |
| | | 59) |
| | | Rev: CTTTTCTGTGAGAATACACTCCAGAGGC (SEQ ID NO. |
| | | 60) |
| 5 | Exon 2 | Fwd: CAGGTTCTGTTCCATAAACTCTGGATTGC (SEQ ID NO. |
| | | 61) |
| | | Rev: CCTATTAAAGCAGTGCTCATGATTGGGTCCG (SEQ ID |
| | | NO. 62) |
| 6 | Exon 3 | Fwd: TGATTATCCTTGCCATTATCCTCCAGGCTCTG (SEQ ID |
| | | NO. 63) |
| | | Rev: ACCAGATAGAACAGACACAGCTACTCTCAG (SEQ ID |
| | | NO. 64) |
| 7 | Exon 4 | Fwd: GGTCTTCAGTTCAAACACCCACAAGC (SEQ ID NO. 65) |
| | | Rev: GCCAAGTGACACTGGTTGTAAATATGC (SEQ ID NO. 66) |
| 8 | Exon 14 | Fwd: GGGCCCATGATAGCCGTCTTTAACAAGC (SEQ ID NO. |
| | | 67) |
| | | Rev: TGTGTCAAATACTTACTTGGCAGAGGTAAATACTTCC |
| | | (SEQ ID NO. 68) |
| 9 | Exon 15 | Fwd: GCTCTTCCTGTTTCAGTCCCCATTAAATGAGG (SEQ ID |
| | | NO. 69) |
| | | Rev: CACCATTGTCTAAGTTCCTAATCTGCAAAGGCC (SEQ |
| | | ID NO. 70) |
| 10 | Exon 16 | Fwd: CCCCCTTACTAATGAGGGCTCTGAGGG (SEQ ID NO. 71) |
| | | Rev: GTTACGCAGTGCTAACCAAGTTCTTTC (SEQ ID NO. 72) |
| 11 | Exon 17 | Fwd: CCCTCAGGACAAGATGCTAACTGTGTGG (SEQ ID NO. |
| | | 73) |
| | | Rev: GGGATGGCTGGCTTACAGCTAGTTTGC (SEQ ID NO. 74) |
| 12 | Exon 18 | Fwd: ACTCCTGGCCTCAAGCCATCCTCTC (SEQ ID NO. 75) |
| | | Rev: GGATTGTGGCACAGAGATTCTGATACTTAC (SEQ ID |
| | | NO. 76) |
| 13 | Exon 19 | Fwd: CTCAGCCTGTTGAATTGGCAATGTCAATGTC (SEQ ID |
| | | NO. 77) |
| | | Rev: CAGAGATAACCAATACATTACCACATCTGACTTGG |
| | | (SEQ ID NO. 78) |
| 14 | Exon 20 | Fwd: GAGACCCTTTGAAGGCAGGCATTTC (SEQ ID NO. 79) |
| | | Rev: GCCAAGTTTAGTTACCAAGACCTACTGATTTC (SEQ ID |
| | | NO. 80) |

TABLE 9

| | | |
|---|---|---|
| Primer | Exon | Sequence |

| | | |
|---|---|---|
| 1 | 2 | (Fwd)TGTAAAACGACGGCCAGTCTCTTCATTTCTGACAACTGAA |
| | | (SEQ ID NO. 81) |
| | | (Rev) |
| | | CAGGAAACAGCTATGACCCTGGCTGGGCTCTTCTATCT |
| | | (SEQ ID NO. 82) |
| 2 | 2 | (Fwd)TGTAAAACGACGGCCAGTCCCACAATCATACTGCTGACA |
| | | (SEQ ID NO. 83) |
| | | (Rev)CAGGAAACAGCTATGACCCTTCCTTCTTTGTGGATCTCTT |
| | | (SEQ ID NO. 84) |
| 3 | 2 | (Fwd)TGTAAAACGACGGCCAGTAAATGCCTCTCTGGAGTGT |
| | | ATTC (SEQ ID NO. 85) |
| | | (Rev)CAGGAAACAGCTATGACCGCACAAAAGAAGCCCTGGATA |
| | | (SEQ ID NO. 86) |
| 4 | 3 | (Fwd)TGTAAAACGACGGCCAGTCTTACCAGCTTGTTCATGTCT |
| | | (SEQ ID NO. 87) |
| | | (Rev)CAGGAAACAGCTATGACCGCACAATACCAGATAGAAC |
| | | AGA (SEQ ID NO. 88) |
| 5 | 4 | (Fwd)TGTAAAACGACGGCCAGTCACAAGCCCTGCTAATCTG |
| | | T (SEQ ID NO. 89) |
| | | (Rev)CAGGAAACAGCTATGACCGCCAAGTGACACTGGTTGT |
| | | A (SEQ ID NO. 90) |
| 6 | 5 | (Fwd)TGTAAAACGACGGCCAGTGGATTTGCACACATAGTTG |
| | | CTAAGT (SEQ ID NO. 91) |
| | | (Rev)CAGGAAACAGCTATGACCGATGACTCACAGCTAAATG |
| | | AGTA (SEQ ID NO. 92) |
| 7 | 6 | (Fwd)TGTAAAACGACGGCCAGTTTGTGACCCTTTTCCCTTTA |
| | | G (SEQ ID NO. 93) |
| | | (Rev)CAGGAAACAGCTATGACCTTTCCAAGCACACCCCAGC |
| | | AAA (SEQ ID NO. 94) |
| 8 | 7 & 8 | (Fwd)TGTAAAACGACGGCCAGTCCTAACCAGAAAATTCCTT |
| | | GGA (SEQ ID NO. 95) |
| | | (Rev)CAGGAAACAGCTATGACCCTTCTCCACACACACACACAA |
| | | (SEQ ID NO. 96) |
| 9 | 9 | (Fwd)TGTAAAACGACGGCCAGTCCCACTTAGGAACCATTGA |
| | | G (SEQ ID NO. 97) |
| | | (Rev)CAGGAAACAGCTATGACCAACAGCATCAAAGCCAGA |
| | | GAT (SEQ ID NO. 98) |
| 10 | 10 | (Fwd)TGTAAAACGACGGCCAGTACTGTGCCTCTGACCTGTAA |
| | | (SEQ ID NO. 99) |
| | | (Rev)CAGGAAACAGCTATGACCAGAGCTCTGAGTAGAACCATT |
| | | (SEQ ID NO. 100) |
| 11 | 11 | (Fwd)TGTAAAACGACGGCCAGTATGGATGTTGCCAAGCTGT |
| | | A (SEQ ID NO. 101) |
| | | (Rev)CAGGAAACAGCTATGACCTTAAAATGCAGTCCACGGT |
| | | TAT (SEQ ID NO. 102) |
| 12 | 12 | (Fwd)TGTAAAACGACGGCCAGTGCAGAACTGTGAAGTGTTAAC |
| | | (SEQ ID NO. 103) |
| | | (Rev)CAGGAAACAGCTATGACCGATGAGCCAAAACCCACAC |
| | | AT (SEQ ID NO. 104) |
| 13 | 13 & 14 | (Fwd)TGTAAAACGACGGCCAGTAAGTGCTACAACCTGTGTA |
| | | GT (SEQ ID NO. 105) |
| | | (Rev)CAGGAAACAGCTATGACCGTCACAACCCACTGAGGTAT |
| | | (SEQ ID NO. 106) |
| 14 | 15 | (Fwd)TGTAAAACGACGGCCAGTTTCCTGTTTCAGTCCCCATT |
| | | AA (SEQ ID NO. 107) |
| | | (Rev)CAGGAAACAGCTATGACCCACTGCTCTGTCAGTTGCTT |
| | | (SEQ ID NO. 108) |
| 15 | 16 | (Fwd)TGTAAAACGACGGCCAGTTTACGCAGTGCTAACCAAGTT |
| | | (SEQ ID NO. 109) |
| | | (Rev)CAGGAAACAGCTATGACCTTTTCCACAAGGGGAAAGT |
| | | GT (SEQ ID NO. 110) |
| 16 | 17 | (Fwd)TGTAAAACGACGGCCAGTATGCTAACTGTGTGGTTTA |
| | | CC (SEQ ID NO. 111) |
| | | (Rev)CAGGAAACAGCTATGACCATGGCTGGCTTACAGCTAG |
| | | TT (SEQ ID NO. 112) |
| 17 | 18 | (Fwd)TGTAAAACGACGGCCAGTCTTGAGCCATTAAGACCAA |
| | | ACT (SEQ ID NO. 113) |
| | | (Rev)CAGGAAACAGCTATGACCGAAACAGATTCCTCCTTGT |
| | | CA (SEQ ID NO. 114) |
| 18 | 19 | (Fwd)TGTAAAACGACGGCCAGTTCTATTTCAGCCACGGGTA |
| | | AT (SEQ ID NO. 115) |
| | | (Rev)CAGGAAACAGCTATGACCAGGAGAAACTCAGAGATA |
| | | ACC (SEQ ID NO. 116) |
| 19 | 20 | (Fwd)TGTAAAACGACGGCCAGTGCCCAAAACAGAAACCGT |
| | | ATTGA (SEQ ID NO. 117) |
| | | (Rev)CAGGAAACAGCTATGACCGACCCTTTGAAGGCAGGCA |
| | | TTT (SEQ ID NO. 118) |
| 20 | 21 | (Fwd)TGTAAAACGACGGCCAGTGGGAGAAGACTCCTACAA |
| | | CCCGAATA (SEQ ID NO. 119) |
| | | (Rev)CAGGAAACAGCTATGACCGTGTGGACTGTTGCTTTGA |
| | | CATAGT (SEQ ID NO. 120) |
| 21 | 21 | (Fwd)TGTAAAACGACGGCCAGTCCTTCTCTGTTGTCATCAG |
| | | AAGAT (SEQ ID NO. 121) |
| | | (Rev)CAGGAAACAGCTATGACCCTGTGATCAAGAAGCCCTC |
| | | AAT (SEQ ID NO. 122) |

TABLE 10

| Exon | Sequence |
|---|---|
| 2 | (Fwd) GACAACTGAACTGCTCTCGCCTTGAACC |
| | (SEQ ID NO. 123) |
| | (Rev) GAGAATATGCAGTGAACCTCCGACTGTATG |
| | (SEQ ID NO. 124) |
| 2 | (Fwd) GACTGGGCCTGTGCTGGAACACC |
| | (SEQ ID NO. 125) |
| | (Rev) GATGAAAGGACTTTGGCTCCCAGGGC |
| | (SEQ ID NO. 126) |
| 2 | (Fwd) GCCCTGGGAGCCAAAGTCCTTTCATC |
| | (SEQ ID NO. 127) |
| | (Rev) CACTCCAGAGGCATTTCCATGTAGGAATGC |
| | (SEQ ID NO. 128) |
| 2 | (Fwd) CATGTCTTTCCCCACAATCATACTGCTGA |
| | (SEQ ID NO. 129) |
| | (Rev) CTTTTCTGTGAGAATACACTCCAGAGGC |
| | (SEQ ID NO. 130) |
| 2 | (Fwd) CAGGTTCTGTTCCATAAACTCTGGATTGC |
| | (SEQ ID NO. 131) |
| | (Rev) CCTATTAAAGCAGTGCTCATGATTGGGTCCG |
| | (SEQ ID NO. 132) |
| 3 | (Fwd) TGATTATCCTTGCCATTATCCTCCAGGCTCTG |
| | (SEQ ID NO. 133) |
| | (Rev) ACCAGATAGAACAGACACAGCTACTCTCAG |
| | (SEQ ID NO. 134) |
| 4 | (Fwd) GGTCTTCAGTTCAAACACCCACAAGC |
| | (SEQ ID NO. 135) |
| | (Rev) GCCAAGTGACACTGGTTGTAAATATGC |
| | (SEQ ID NO. 136) |
| 14 | (Fwd) GGGCCCATGATAGCCGTCTTTAACAAGC |
| | (SEQ ID NO. 137) |
| | (Rev) TGTGTCAAATACTTACTTGGCAGAGGTAAATACTTCC |
| | (SEQ ID NO. 138) |
| 15 | (Fwd) GCTCTTCCTGTTTCAGTCCCCATTAAATGAGG |
| | (SEQ ID NO. 139) |
| | (Rev) CACCATTGTCTAAGTTCCTAATCTGCAAAGGCC |
| | (SEQ ID NO. 140) |
| 16 | (Fwd) CCCCCTTACTAATGAGGGCTCTGAGGG |
| | (SEQ ID NO. 141) |
| | (Rev) GTTACGCAGTGCTAACCAAGTTCTTTC |
| | (SEQ ID NO. 142) |
| 17 | (Fwd) CCCTCAGGACAAGATGCTAACTGTGTGG |
| | (SEQ ID NO. 143) |
| | (Rev) GGGATGGCTGGCTTACAGCTAGTTTGC |
| | (SEQ ID NO. 144) |
| 18 | (Fwd) ACTCCTGGCCTCAAGCCATCCTCTC |
| | (SEQ ID NO. 145) |
| | (Rev) GGATTGTGGCACAGAGATTCTGATACTTAC |
| | (SEQ ID NO. 146) |
| 19 | (Fwd) CTCAGCCTGTTGAATTGGCAATGTCAATGTC |
| | (SEQ ID NO. 147) |
| | (Rev) CAGAGATAACCAATACATTACCACATCTGACTTGG |
| | (SEQ ID NO. 148) |
| 20 | (Fwd) GAGACCCTTTGAAGGCAGGCATTTC |
| | (SEQ ID NO. 149) |
| | (Rev) GCCAAGTTTAGTTACCAAGACCTACTGATTTC |
| | (SEQ ID NO. 150) |

Also disclosed are methods for identifying a specific mutation in c-met in a sample. For example, disclosed are methods for identifying a specific mutation in c-met in a sample, wherein the mutation is one that results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I relative to wild type c-met, said method comprising contacting the sample with a nucleic acid comprising the sequence of any of the primers/probes listed in Tables 7-10. Also disclosed are methods for identifying a specific mutation in c-met in a sample, wherein the mutation is in exons 2, 3, 4, 7, 9, 13, 14, 15, 17, 19, 20 and/or 21, and/or their flanking introns, wherein the mutation affects exon splicing and/or protein structure, said method comprising contacting the sample with a nucleic acid comprising the sequence of any of the primers/probes listed in Tables 7-10.

Further disclosed are methods of detecting presence of a mutated c-met in a cancer. For example, disclosed are methods of detecting presence of a mutated c-met in a cancer, the method comprising contacting a sample suspected or known to comprise mutated c-met with a nucleic acid comprising the sequence of any of the primers/probes listed in Table 7-10. Also disclosed are methods of detecting the presence of a mutated c-met in a cancer, the method comprising contacting a sample suspected or known to comprise mutated c-met with an antigen binding agent capable of binding to a peptide that contains an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I relative to wild type c-met, wherein binding of the agent, or lack thereof, is indicative of presence or absence of a c-Met polypeptide comprising a mutation of at least a portion of exons 2, 3, 4, 7, 9, 13, 14, 15, 17, 19, 20 and/or 21.

Also disclosed are methods for detecting a cancerous disease state in a tissue. For example, disclosed are methods for detecting a cancerous disease state in a tissue, said methods comprising determining whether a sample from a subject suspected of having a cancer comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I, wherein detection of said mutation is indicative of presence of a cancerous disease state in the subject. Also disclosed are methods for detecting a cancerous disease state in a tissue, said methods comprising determining whether a sample from a subject suspected of having a cancer comprises a mutation in a nucleic acid sequence encoding human c-Met, wherein the sequence is mutated in exons 2, 3, 4, 7, 9, 13, 14, 15, 17, 19, 20 and/or 21, and/or their flanking introns, wherein the mutation affects exon splicing and/or protein structure, wherein detection of said mutation is indicative of presence of minimal residual cancer.

Also disclosed are methods of assessing a subject's increased susceptibility to develop a cancer comprising determining whether the sample comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I.

Also disclosed are methods of treating a cancer comprising administering an effective amount of one or more of the disclosed compositions alone or in combination with at least one chemotherapeutic agent. Also disclosed are methods of treating a cancer comprising administering an effective amount of one or more of the disclosed composition in conjunction with radiation therapy. Also disclosed are methods of treating a cancer comprising administering an effective amount of one or more of the disclosed compositions alone or in combination with at least one chemotherapeutic agent in conjunction with radiation therapy.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-II (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISEME®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELB AN®); platinum; etoposide (VP-16); ifosf amide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Disclosed herein are kits that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. Also disclosed are kits comprising a composition of the invention, and instructions for using the composition to detect mutation in human c-Met at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I. Further disclosed are kits comprising a composition of the invention, and instructions for using the composition to detect human c-Met comprising a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

The transgenic animals described above can also be used in any of the methods described herein. For example, the transgenic animals described herein can be used to identify a cancer that is susceptible to treatment with a c-met inhibitor, said method comprising determining whether a cancer sample from a subject comprises a mutation in a nucleic acid sequence encoding human c-met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010L Q1029E, S1167N, T1275I, P1300S, P1301S, and/or V1333I.

The transgenic animals described herein can also be used to identify a cancer that is susceptible to treatment with a c-met inhibitor, said method comprising determining whether a cancer sample from a subject comprises a mutation in a nucleic acid sequence encoding human c-met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

The transgenic animals described herein can also be used to determine the responsiveness of a cancer in a subject to treatment with a c-met inhibitor, said method comprising determining whether a cancer sample from a subject who has been treated with the c-met inhibitor comprises a mutation in a nucleic acid sequence encoding human c-met, wherein the mutation results in an amino acid change at position L211W, T230M, S244P, L229F, F253S, S323G, A347T, E355K, R359Q, M362T, M431V, N454I, S470L, 1852F, N948S, S1058P, T1010I, Q1029E, S1167N, T1275I, P1300S, P1301S, and/or V1333I, wherein absence of the mutated nucleic acid sequence is indicative that the cancer is responsive to treatment with the c-met inhibitor.

The transgenic animals described herein can also be used to determine the responsiveness of a cancer in a subject to treatment with a c-met inhibitor, said method comprising determining whether a cancer sample from a subject who has been treated with the c-met inhibitor comprises a mutation in a nucleic acid sequence encoding human c-met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure, wherein absence of the mutated nucleic acid sequence is indicative that the cancer is responsive to treatment with the c-met inhibitor.

The transgenic animals described herein can also be used to identify a cancer that is susceptible to treatment with a c-met inhibitor, said method comprising determining whether a cancer sample from a subject comprises a mutation in a nucleic acid sequence encoding human c-met, wherein the mutation results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I.

The transgenic animals described herein can also be used to identify a cancer that is susceptible to treatment with a c-met inhibitor, said method comprising determining whether a cancer sample from a subject comprises a mutation in a nucleic acid sequence encoding human c-met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

The transgenic animals described herein can also be used to determine the responsiveness of a cancer in the transgenic animal to treatment with a c-met inhibitor, said method comprising determining whether a cancer sample from a subject who has been treated with the c-met inhibitor comprises a mutation in a nucleic acid sequence encoding human c-met, wherein the mutation results in an amino acid change at position L211W, T230M, S244P, L229F, F253S, S323G, A347T, E355K, R359Q, M362T, M431V, N454I, S470L, 1852F, N948S, S1058P, T1010I, Q1029E, S1167N, T1275I, P1300S, P1301S, and/or V1333I, wherein absence of the mutated nucleic acid sequence is indicative that the cancer is responsive to treatment with the c-met inhibitor.

The transgenic animals described herein can also be used to determine the responsiveness of a cancer in the transgenic animal to treatment with a c-met inhibitor, said method comprising determining whether a cancer sample from a subject who has been treated with the c-met inhibitor comprises a mutation in a nucleic acid sequence encoding human c-met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure, wherein absence of the mutated nucleic acid sequence is indicative that the cancer is responsive to treatment with the c-met inhibitor.

Other examples of uses of the disclosed transgenic animals can be seen in the Examples below.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### Example 1

### Expression and Gene Amplification of c-Met in NSCLC Cell Lines

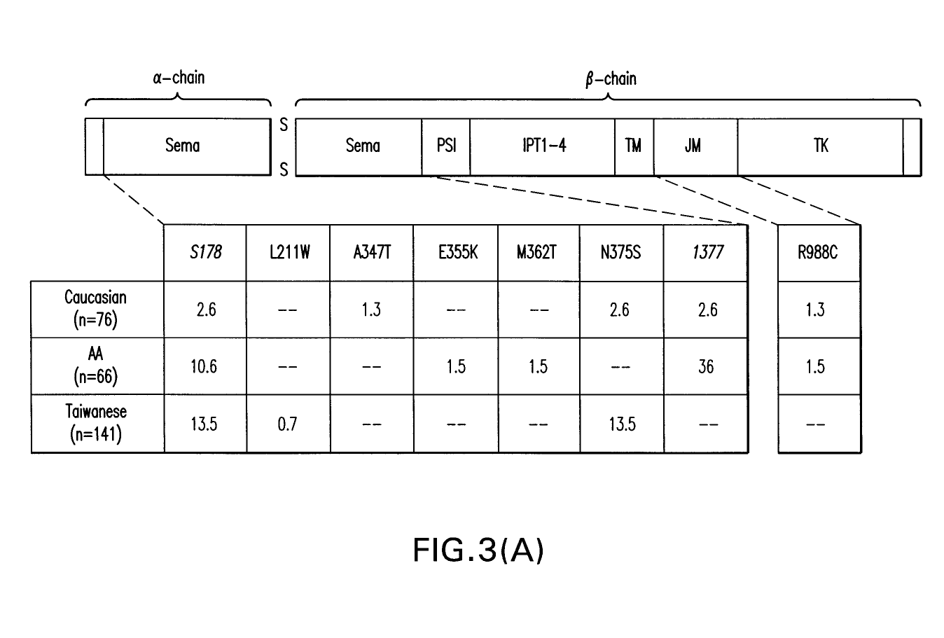

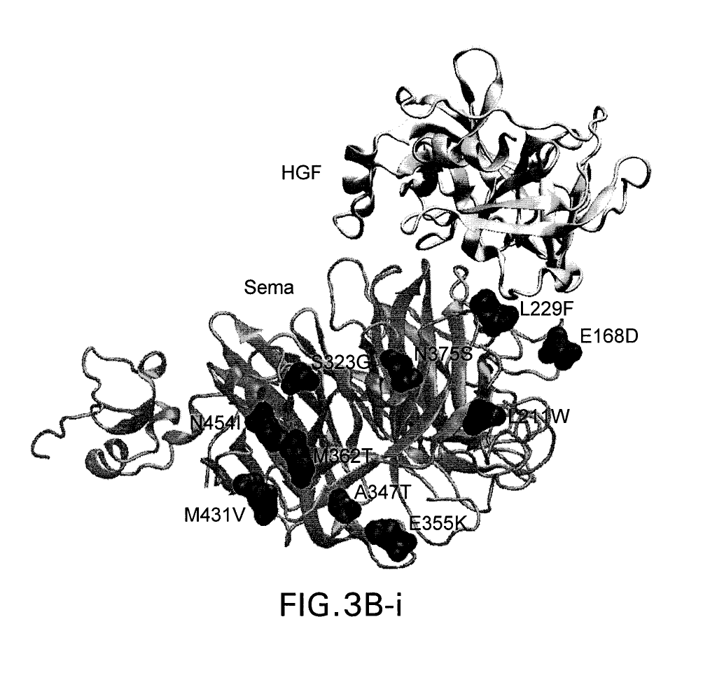

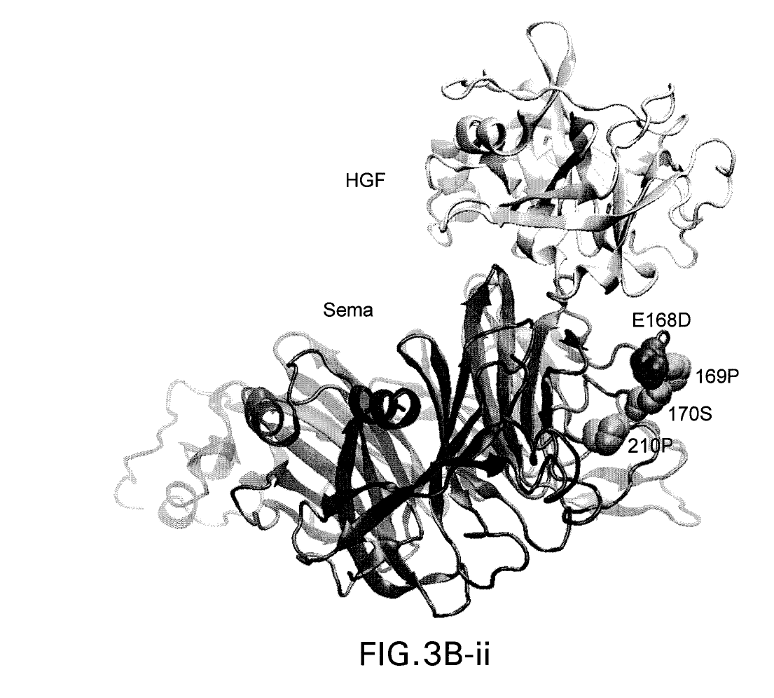

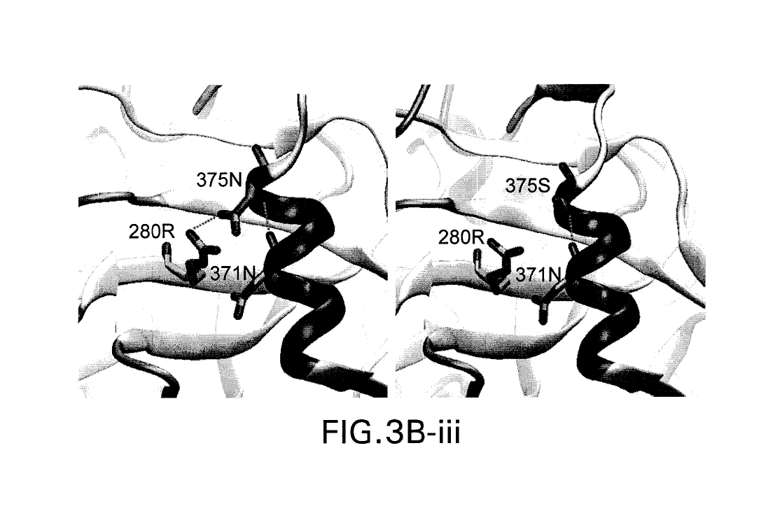

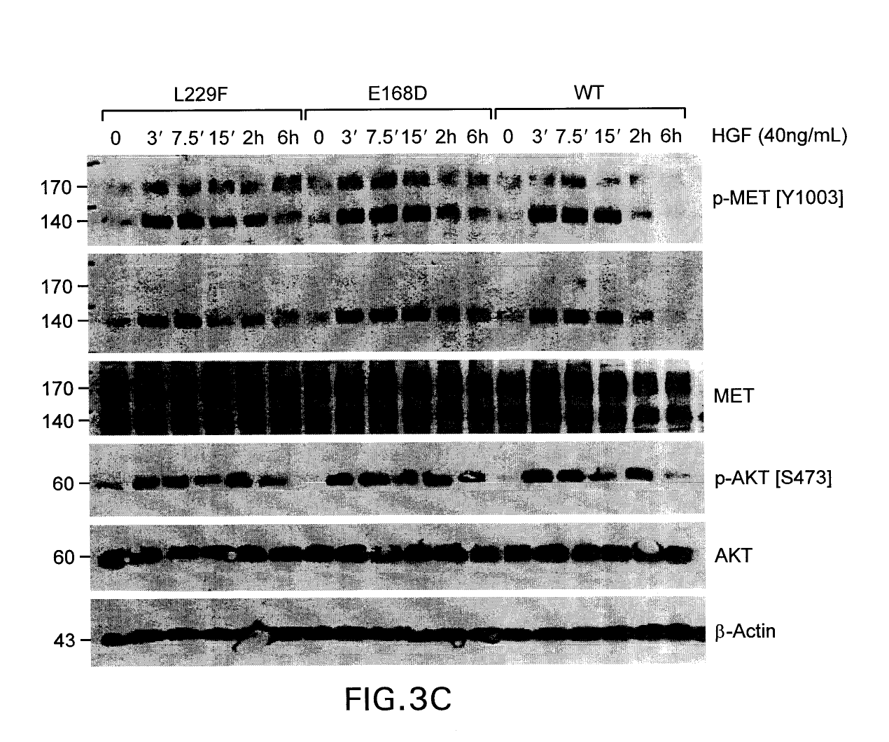

The expression of c-Met RTK oncogene was studied in various NSCLC cell lines using standard immunoblotting with the antihuman polyclonal c-Met antibody (C-12, Santa Cruz). It was found that there was high expression of c-Met in most of the NSCLC cell lines (−75%), including H441, SKLU-1, H1993, A549, H1838 and H358 except H-522 and H661 (FIG. 3). This was also evident in the lung tumor tissue immunoblotting and paraffin embedded lung cancer tissue micro array Immunohistochemical analysis. Amplification of the c-Met region was further analyzed using Fluorescent in situ hybridization (FISH) technique to determine the degree or correlation between amplification, mutation, and expression of c-Met within the lung cancer cell lines. The analysis revealed amplification of the c-Met region on chromosome 7p11.2 to copy numbers above 15 in two (out of eight; 25%) NSCLC cell lines (H1993, H1838), and this correlated with high expression of c-Met. A EGFR probe was used as a control and was found to have a similar frequency of amplification in NSCLC.

Tissue Array (TTA) at UoC and RPCI: Expression of c-Met

A TTA of different solid tumor types and sub types was previously constructed (Beecher Instrument ATA-27 Automatic Arrayor, Sun Pairie, U.S.A.) for expression and linked analysis studies. Tumors showed a differential range of c-MET expression and subcellular localization. It was also found that phospho-c-Met is heavily overexpressed in lung cancer in comparison to other tumors. In the current studies, NSCLC tissue micro arrays were specifically utilized for IHC analysis for the expression of c-Met, its activated phospho-c-Met and also in the context of tumor invasive front or other clinical outcome correlations. The results revealed invasive fronts of the tumor moving towards the adjacent normal tissue. Cytoplasmic and membranous expression of c-Met and phospho-c-Met was quantified manually in each core at ×400 magnification by two pathologists and with Automated Cellular Imaging System (ALIS, Clarient, Calif.).

### Preferential Activation of Phospho-Met Along NSCLC Tumor Invasive Front

A preferential expression of the activated phospho-Met in the tumor cells located in the invasive front of the NSCLC tissues, both in the case of pY1003-Met and pY1230/1234/1235-Met has also been observed. IHC staining of archival adenocaricnoma tissue with antibody against pY1003-Met and with antibody against pY1230/1234/1235-Met were both performed. Such fidings were also observed in other histologies of NSCLC (See Ma, et al., Cancer Res, 65, (4), 1479-88 (2005)). Such findings are indicative of a key role c-Met expression plays in NSCLC tumor progression, cell proliferation, invasion and eventual metastasis.

Mutation (Germline/Somatic) of c-Met in NSCLC

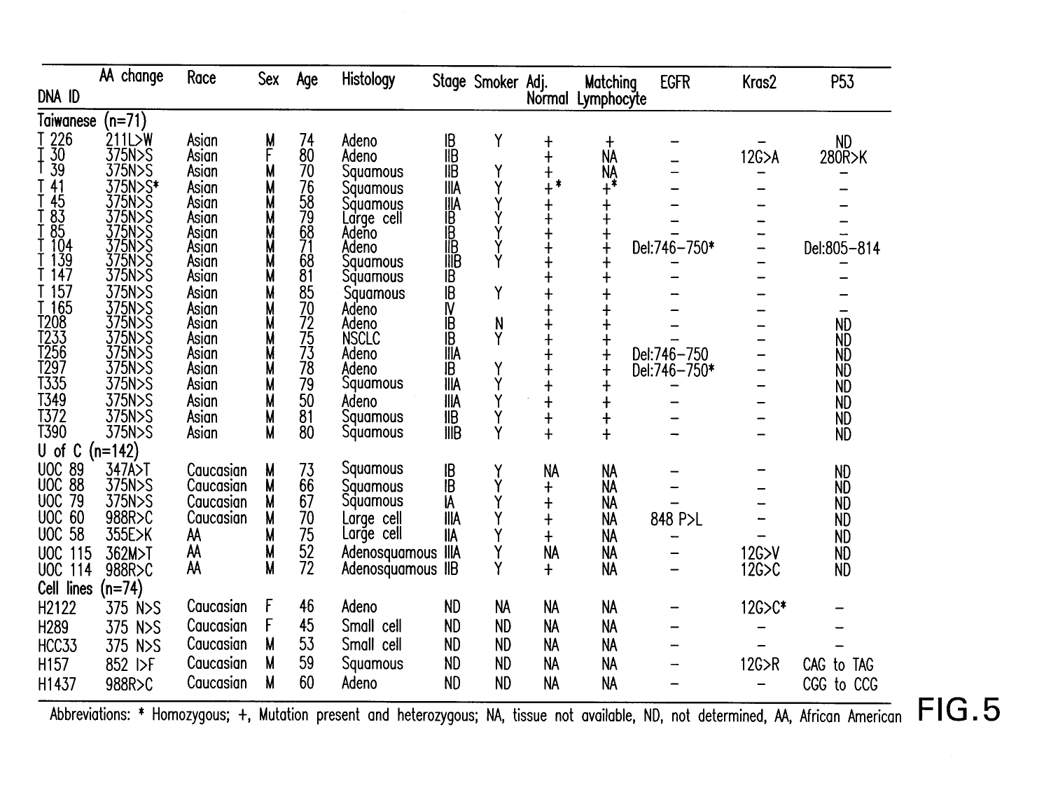

The frequency and type (somatic/germline) of the various c-Met mutations, described elsewhere herein, among the DNA samples obtained from different ethnic groups (FIG. 5) including Asian (Taiwanese), Caucasian and African Americans (AA) were also evaluated. DNA from tumor tissues, the adjacent normal tissue, and peripheral blood DNA were evaluated for c-Met mutational analysis using a multiplex PCR method.

Mutations of E168 D, L229F, S323G, A347T, E355K, V362T, N375S, R988C, T1010I and S1058P and exon 14 deletion splice variant of c-Met were identified and clustered in the sema and JM domain. N375S mutation in the HGF binding sema domain was found in 14% of Taiwanese lung cancer patients. African-Americans (n=66) did not show N375S mutation. No mutations were found in the tyrosine kinase domain of lung cancer samples. A negative regulatory JM domain mutations R988C and T1010I and S1058P were detected in lung cancer patients DNA obtained from Caucasian and AA and was identified as a germline mutation. The relationship between mutations of c-Met, p53, K-Ras and EGFR were also evaluated. The majority of the missense mutations identified in lung cancers are clustered with in the HGF binding Sema and regulatory Juxtamembrane (JM) domains.

Specific Inhibition of c-Met Signaling Using Small Molecule c-Met Inhibitors

The effectiveness of siRNA against c-Met to inhibit NSCLC cell lines was previously reported in Ma, et al., Cancer Res, 65, (4), 1479-88 (2005). Several other ATP-competitive small molecule selective inhibitors of c-Met, namely SU11274 (1^{st} generation), PHA665752 (2^{nd} generation) made available from Pfizer, Inc. and Compound X (3^{rd} generation) were used for studies of c-Met inhibitory strategies.

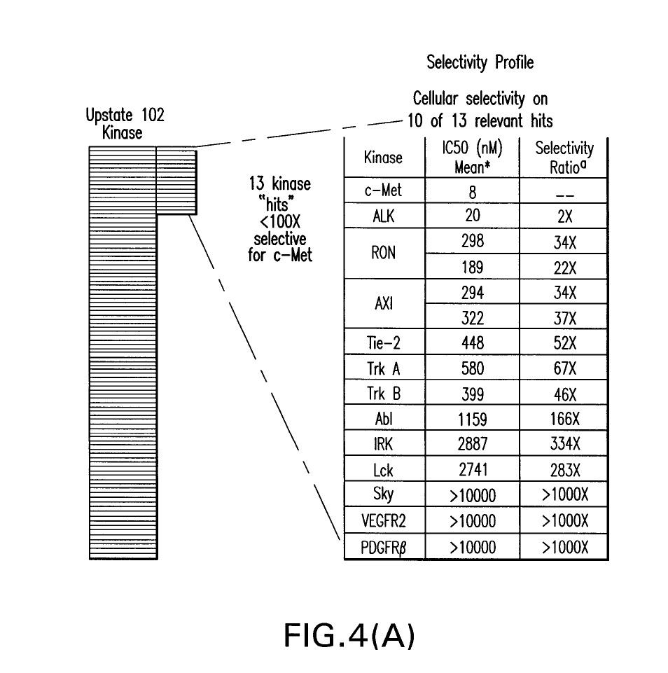

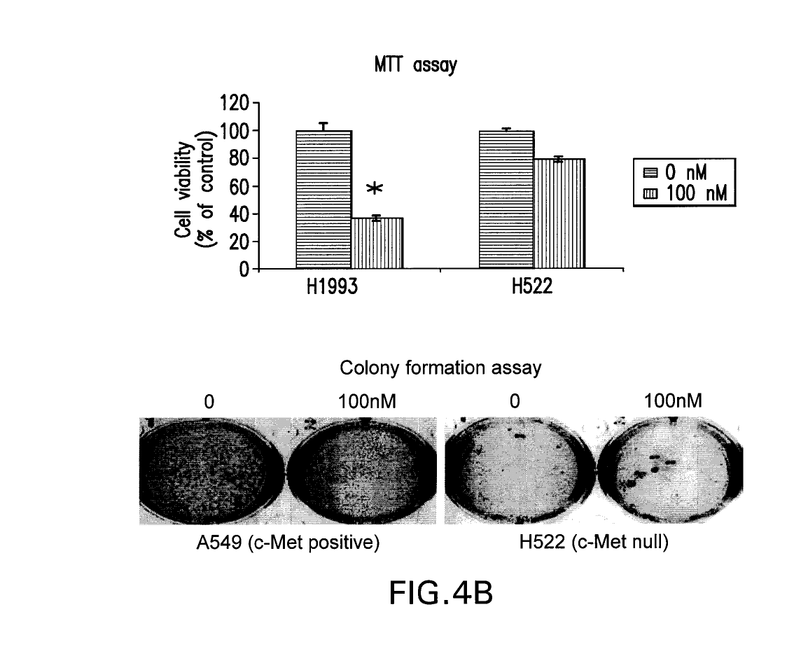

Compound X was used to test for its inhibitory effects on NSCLC cell lines (FIG. 4). It was apparent that the c-Met selective inhibitor Compound X is effective against the viability of highly c-Met amplified (H1993) and c-Met overexpressing A549 cells. In the c-Met null H522 cells, c-Met inhibitor (Compound X) effect was not appreciable.

Results also showed that Compound X inhibited HGF stimulated migration and invasion of NSCLC cells. Prestarved c-Met overexpressed NSCLC cells were stimulated with or without HGF (40 ng/ml, 7.5 min), and in the absence or presence of c-Met inhibitor Compound X (4-256 nm). Cells were stained and counted for the drug inhibition on HGF stimulated migration and invasion. A decrease in migration and invasion was observed.

In vitro Inhibition of the HGF/c-Met Signaling Pathway in NSCLC by Small Molecule Inhibitor

Using Compound X, the inhibitory effects on the HGF/c-Met signaling pathway in the c-Met over-expressing H441 cells was further defined. Prestarved c-Met overexpressed NSCLC cells were stimulated with or without HGF (40 ng/ml, 7.5 min), and in the absence or presence of c-Met inhibitor. Cell lysates were prepared at the completion of the drug treatment and HGF stimulation, separated by 7.5% SDS-PAGE and then immunoblotted using specific antibodies. HGF-induced tyrosine phosphorylation of cellular proteins in the NSCLC cells was inhibited by Compound X (100 nM). HGF-induced phosphorylation of the autophosphorylation site of c-Met (pY1230/1234/1235-Met) was also abrogated by the met inhibitor. Tyrosine phosphorylation at the pY1349-Met site was also blocked by the c-Met inhibitor. In addition, specific HGF-induced phosphorylation of AKT, Raf, MEK1/2, ERK1/2 and p90RSK was inhibited by Compound X in NSCLC cell lines, indicating the inhibition of the pathway by the drug.

Specific In Vitro Inhibition of HGF Induced Cell Proliferation and c-Met Signaling Pathway in NSCLC Using Anti-Met 5D5 Monoclonal Antibody

An anti-Met 5D5 monoclonal antibody was utilized to test for c-Met inhibitory effects on NSCLC cell lines. Cells from two NSCLC cell lines (H1838 and H522) were seeded onto 96-well plates and pretreated with the indicated concentrations of the 5D5 and vehicle control with or without HGF. Cell growth inhibitory effect of anti-Met 5D5 was assayed using MTT assay and signaling by immunoblotting. Treatment with anti Met 5D5-Fab caused an inhibition of HGF induced proliferation in NSCLC cells. The data obtained indicated that the anti-Met 5D5 monoclonal antibody is selectively effective against the c-met amplified H1838 cells. In the c-Met null H 522, there was no significant proliferation with HGF as well as no inhibition with 5D5. The inhibitory effects of the anti-Met 5D5 monoclonal antibody on the HGF/c-Met signaling pathway was also observed in the c-Met overexpressing H1838 cell line. HGF-induced phosphorylation of the autophosphorylation site of c-Met (pY1230/1234/1235-Met) was abrogated by 5D5. Tyrosine phosphorylation at the pY1003-Met site within the JM domain was also blocked by the 5D5. In addition, specific HGF-induced AKT was inhibited by 5D5 in c-Met overexpressing NSCLC cells.

### Example 2

### In Vivo Inhibition of c-Met in Mouse Model

Inhibition of Tumor Growth by Prototype c-Met Inhibitor in a Tumor Xenograft Model

An in vivo nude mouse xenograft model (engineered to form HGF autocrine loop) was generated to test against the small molecule c-Met inhibitors. Nude mice were subcutaneously injected with equal numbers of NSCLC H441 cells and regular tumor size measurements done in follow up. Intra-peritoneal injection of either vehicle control or Compound X was also performed. Reduced plasma levels of soluble Met (sMet) in tumor xenograft models treated with Compound X. sMet levels were measured by ELISA *p<=0.05 (Middle panel). Compound X inhibited c-Met/HGF signaling and its downstream pathways in H441 xenograft. The results of inhibition of NSCLC H441 cells xenografted onto the animals using the prototype inhibitor Compound X were obtained. There was inhibition of phospho-c-Met after inhibitor treatment in a dose dependent manner. In addition, inhibition of the activation of c-Met and downstream molecules was observed in the presence of PF in a H441 cell model engineered to form and HGF autocrine loop. Inhibition of phosphorylation of the autophosphorylation site of c-Met (pY1230/1234/1235-Met) by met inhibitor was also observed. Tyrosine phosphorylation at the pY1003-Met site within the JM domain was also blocked by the c-Met inhibitor along with tumor growth. In addition, phospho AKT (Thr 308), phospho MAPK 42/44 (T202/Y204) and Phospho Gab1 (Y627), was inhibited by Compound X in xenograft, indicating the inhibition of the pathway by the drug. There was a decrease in plasma levels of c-Met in animal xenograft models treated with c-Met inhibitor suggesting that soluble met (sMet) could potentially serve as a biomarker of anti-Met activity in our clinical trials.

### Example 3

c-Met is a RTK that is an important molecule in the pathogenesis and metastasis of NSCLC. Data indicates that c-Met is expressed in NSCLC cell lines and overexpressed in some NSCLC tumor tissues. As described above and elsewhere herein, potential mutations of c-Met in the tyrosine kinase domain, juxtamembrane (JM) domain, and Sema domain have been evaluated in some lung cancer samples. As described above, several mutations in the JM domain and the Sema domain of c-met have been identified in lung cancer samples, but not in the tyrosine kinase domain. As such, determination of the presence and effect of other c-met gene mutations in lung cancer can be observed.

### Sample Acquisition and Study Numbers

To begin, prospective and retrospective acquisition of tissue and bodily fluid samples from patients with lung cancer can be obtained. Tumor slides have been pre-reviewed by a pathologist and selected according to their suitability for tissue array production. From 1987-2004, 1,083 samples have been identified, reviewed and entered into a tissue bank. Array building of these samples can also be performed. Demographic, clinical, and outcomes data have been entered into a computer file for nearly one-half of the cases (497). Thirty eight percent of these samples are from African-Americans.

In addition 250 paraffin embedded tissue samples from Chinese NSCLC patients from Taiwan have been obtained. At least 50 additional Taiwanese NSCLC samples can be obtained along with lymphocyte DNA for germline studies in normal Taiwanese controls.

All samples have associated clinical information (survival, demographics, stage, histology, etc.) and genomic DNA and RNA, as well as associated genomic DNA from adjacent normal lung and/or circulating lymphocytes. Lastly through the CALGB access to 467 serum samples from patients with NSCLC stages I, II, IIIA, undergoing surgery (with representative histologic distribution) has been granted.

Immunohistochemistry can be performed on a total of 900 samples, 300 from each of the three ethnic groups. Of these, 600 can be selected for mutational analysis and, of these, 300 for gene amplification studies. This scheme maximizes the number of cases with data available on all three types of measures for multivariable analyses. Power estimates based on these numbers for each of the sub-aims are provided below.

Determination of the Mutations of c-Met in Different Ethnic Groups.

Preliminary analysis on c-Met alterations (mutations) has been performed on different ethnic subtypes. In particular, for the complete c-Met analysis for 142 Taiwanese NSCLC, 232 NSCLC from Caucasians, and 65 from African-Americans, the following preliminary conclusions can be made: (1) Taiwanese NSCLC samples have only one specific mutation, N375S, in c-met gene. The frequency is 14.8% (21/142). Interestingly this mutation was detected in the associated lymphocyte DNA (suggestive of a germline mutation; and not found in the NCBI-SNP database). This is analogous to HPRCC; (2) African-Americans do not have the N375S mutation (0/66). The three mutations identified so far are all non N375S; (3) Caucasians appear to have several mutations within the semaphorin domain and the juxtamembrane domain (including among others the N375S mutation). The rate of the N375S mutation is 4.5% (3/66) and in general the mutation rates of c-Met are 7.5% (5/66), with 80% in the SEMA domain and 20% are in the JM domain (4). Thus far, no mutations of the tyrosine kinase domain have been identified in any lung cancer sample set; (5) both somatic and several germline mutations of c-Met have been identified; and (6) unlike the mutual exclusivity of EGFR mutations with K-ras mutations in NSCLC, a simple relationship between c-Met mutations, EGFR mutations, and K-ras/p53 mutations is not appreciable.

In order to further examine these findings, genomic DNA and RNA from tumor samples and the adjacent normal tissue can be obtained (to determine germline versus somatic mutations). c-Met can then be sequenced and any mutations identified can be catalogued.

The genomic DNA from the tumor and normal tissues can be prepared in standard fashion (Di Renzo et al., Clin Cancer Res, 1, (2), 147-54 (1995)) and genomic DNA will be used to amplify across each exon and some flanking intronic regions. Standard PCR, multiplex PCR, and sequencing techniques can be used to obtain the sequences (as previously described by Di Renzo, et al. (see above) Sequencing can be performed using dye-primer chemistry and the Prism 377 DNA Sequencer (Applied Biosystems). Sequencing can be performed with the forward coding strand with confirmation of c-Met genomic or cDNA alterations performed by sequencing of the reverse strand as well.

The frequency of mutations can be compared using a two degree-of-freedom (df) chisquare test, followed by pairwise comparisons if the two df test is statistically significant (Cochran et al., Biometrics, 10, 10:417-451 (1954)). For pairwise comparisons the sample size will provide 90% power to detect a difference of approximately 10% if the true percentages are in the range of 5 to 15%, and 90% power to detect a difference of 7% if the true percentages range from <1 to 10%, based on a two-sided test at the 0.05 significance level.

### Determination of the Incidence of Germline N375 S Mutations in Chinese (Taiwan) NSCLC Cancer Patients and Age/Smoking Matched Normals

It is thought that the N375S germline mutation predisposes to the development of NSCLC. Precedence for this is provided by the frequent (70% in Rottweiler, 5% in dogs in general) presence of a germline mutation in the c-Met JM domain. The example of Rottweiler dogs shows the frequent development of tumors and gain-of-function effect of this mutation (Liao et al., Anim Genet, 37, (3), 248-52 (2006)). Also, in hereditary papillary renal cell carcinomas, there are germline mutations of c-Met.

Mutational analysis can be performed as described above. Two hundred fifty NSCLC samples from Taiwan have been transferred and banked—200 of these can be used. The controls can be frequency matched to the cases by age (<50, 50-59, 60-69, 70 years or older) and lifetime smoking (never, pipe and cigar only, cigarettes <20 pack-years, 20-40 pack-years, >40 pack-years).

The frequency of germline mutations can be compared between NSCLC and normal controls using a chisquare test. Evaluation of 200 cases and 400 controls can provide 80% power to detect a 15% vs. 7.2% difference or an odds ratio of 2.3.

Determination of the Differences in c-Met Mutations, Amplification, and c-Met Expression Between the Different Histologies and Stages of NSCLC

The c-Met alterations, amplification, and expression levels can be obtained in the different histologies (adenocarcinomas, SCC, large cell carcinomas, and bronchiolo-alveolar carcinoma) and different stages of NSCLC. As discussed above, c-Met expression levels can be determined in 900 cases; mutational analysis can be performed in 600 of these cases; and gene amplification in 300. From a current database, the breakdown by histology and stage preliminarily is as follows: 229 (46%) adenocarcinoma, 168 (34%) squamous cell, 24 (5%) large cell, 14 (3%) bronchiolo-alveolar, and 62 (12%) other; 100 (24%) stage 1, 32 (8%) stage II, 149 (36%) stage III, and 130 (32%) stage IV, with 86 still under review (omitted from percentage calculations).

c-Met mutation rates and gene amplification can be compared across the histological types and among the different stages using a chi-square test. Expression levels recorded on the ordinal scale will be analyzed using the nonparametric, Kruskal-Wallis test followed by Dunn's test and by chisquare tests after dichotomization into strong vs. negative/weak staining.

### Example 4

As described above, the various mutations, amplification, and expression (including activation) of c-Met in NSCLC have been examined. Since these mutations are unique and some are potentially germline, they may potentially enhance the risk of lung cancer. The biological and biochemical effects of wild type c-Met and altered c-Met on normal human bronchial epithelial cells and NSCLC cells can be assessed. The relevant biological functions include cell viability, survival, cell motility and migration, and scatter function. The biochemical functions such as the interactions of c-Met with HGF and receptor function can also be determined.

### Determination of the Biological Effects on Various NSCLC and Normal Cells

Several novel mutations and alterations of c-Met in NSCLC have been described above. Also described above was the generation of constructs using IRES2-EGFP; however, the pAcGFP1-N1 and pDsRed-Monomer-N1 vectors (Clontech) have been modified into Gateway destination vectors (Invitrogen) for high expression in mammalian cells. These vectors have the advantage of having monomer fluorescence proteins as well as easily transfectable selectivity with G418. The efficiency of transfections was 80-90%. In addition, full length wild-type c-Met, mutations of c-Met (E168D, L229F, S323G, A347T, E355K, N375S, M382S, M431V, V466E, N454I, S470P, R988C, T1010I, R988C/T1010I double mutations, S1058P, and JM deletion), the JM domain alone, and (as controls) the M1268T mutant of c-Met, the Tpr-Met fusion and the mock transfected cells can also be generated. The control M1268T mutant has been well characterized in sporadic renal papillary cancer with strong auto-phosphorylation and biological activity (Miller et al., Proteins, 44, (1), 32-43 (2001)). Tpr-Met has also been shown to have potent tyrosine kinase activity and enhanced biological activity and will serve as a control.

The cell lines choosen for this analysis were: H522 or H661 (NSCLC) and NHBE (normal human bronchial epithelial) cells. The H661 NSCLC cell line was chosen since it has no detectable expression of c-Met and this would serve as an excellent model for studying the behavior of wild-type and mutant c-Met in the context of NSCLC. Preliminary data indicated, using the vector alone, a transfection efficiency rate with lipofectamine of approximately 60%. NHBE cells can also serve as primary cells for studying c-Met and mutations in the context of potential lung biology, signal transduction and lung carcinogenesis whereas, for transient studies, NHBE cells can be utilized.

To facilitate the functional analysis of c-Met mutations in normal lung and lung cancer, immortalized primary NHBE cells, which have overcome replicative senescence and also culture crisis can be utilized in vitro. Immortalized NHBE cells were generated through the successive introduction of the Simian Virus 40-Early Region (SV40-ER and the telomerase catalytic subunit hTERT as described in Lundberg et al., Oncogene, 21, (29), 4577-86 (2002). Primary human airway epithelial cells immortalized in this way have been shown to be responsive to malignant transformation by an introduced H-ras or K-ras gene. Transfection of immortalized NHBE cells can be achieved by the Retroviral Expression System, Retro-X™ System (Clontech) which can readily yield transduction of nearly 100% of cells with retrovirus-mediated gene transfer and subsequent creation of stable cell lines.

Additionally, both transient transfectants and stable transfectants with G418 selection can be generated. As controls, the parental cell lines can be used as well as vector alone transfected cell lines. Clonal populations of low, medium, and high expressor of the c-Met constructs can be used to determine the effect of expression on the various biological/biochemical functions proposed below.

### Example 5

### Functional Biological Aspects of c-Met Mutations

There are a number of biological functional assays that can be performed for the cell lines generated using the methods described above. It is also relevant to study cell growth and viability, cell survival, and transformation of the c-Met mutations to understand the biology of the c-Met mutations. Cell motility/migration as well as scatter function can also be assessed.

### Cell Growth and Viability

The cell growth and viability of the cell lines generated can be assessed with trypan blue exclusion and MTS assay. A dye conversion assay (MTS) can be used to study the various cell lines and thus such an assay can be used to study the kinetics (over 72 hours) and dose-response effects of HGF (0-100 ng/ml). The assays can be conducted with and without FCS to determine the effect of serum-stimulated growth. As an alternative strategy (also for many of the assays below), HGF could be transfected concomitantly with the c-Met constructs to determine the endogenous effects of HGF.

### Cell Survival and Apoptosis

Cell survival assays can also be performed for the cell lines generated above. The cell survival of H661 NSCLC cells and NHBE cells with the various transfected c-Met constructs can be deduced, since this will closely reflect the NSCLC/lung cancer behavior. Initially, the cells can be serum deprived and a survival curve with and without HGF can be determined via a trypan blue exclusion assay (a concentration of 100 ng/ml HGF is reasonable for survival in NSCLC cell lines). Several steps are needed to perform the analysis. Briefly, after collecting floating cells, attached cells can be exposed to 0.05% trypsin, 0.02% EDTA. Trypsin can be inactivated by soybean trypsin inhibitor (Sigma). All the attached and detached cell populations can then be combined to determine the proportion of dead cells. Trypan blue (Life Technologies, Inc.) can then be mixed with cells (1:4), and trypan blue exclusion by living cells will be scored using phase contrast microscopy. This traditional counting of cells by eye is subject to bias. As an alternative strategy, simultaneously, the counting of dead cells as well as apoptotic cells can be counted via the measurement of propidium iodide uptake on flow cytometry.

The ability of the transfected cells to undergo (or have reduced) apoptosis (with and without HGF) can also be examined. The H661 and NHBE cell lines generated can be utilized in this study and apoptosis can be determined by determining the sub-G1 population detected on cell cycle analysis, DNA laddering, staining of cells with annexin V labeled with FITC, staining of cells with uptake of vital stain 7-amino-actinomycin D (7-AAD), and caspase-3 activation. Caspase-3 activation can be determined by immunoblotting, colorimetric assay (BioVision assay kit) based on spectrophotometric detection of the chromophore p-nitroanilide (pNA) after cleavage from the labeled substrate DEVD-pNA, and fluorometric assay using the Ac-DEVD substrate (UBI).

### Transformation

Clonogenic assays can also be used to determine cell survival/transformation potency of NSCLC transfected with various c-Met constructs. NSCLC cells such as H661 are able to form colonies in an agarose medium. Soft agarose assays can also be performed for transformation ability in the various cell lines generated (Andoniou et al., Oncogene, 12, (9), 1981-9 (1996)). Recently it has been shown that these clonogenic assays can be performed in a more efficient manner. In a study by Ke et al, anchorage-independent growth and transformation for the cell lines was preformed in an alternative 96-well soft agar growth assay that could function as a replacement for the traditional soft agar colony formation assay method (Ke et al., Biotechniques, 36, (5), 826-8, 830, 832-3 (2004)).

### Nude Mouse Modeling

The in vitro cell line model is useful in studying the behavior of NSCLC, however, the cell line model may not be sufficient to show tumorigenic potential of the various mutations. Thus, initially, the tumor activity in a nude mouse model can be determined using the transfected H661 cell lines with c-Met constructs (Andoniou et al., Oncogene, 12, (9), 1981-9 (1996)). To do this, male athymic nude mice can be injected subcutaneously, into the right flank with 1×10⁷ of human transfected NSCLC cells/mouse in 0.1 or 0.2 ml of PBS. Animals can be examined daily, and differential growth of tumors measured up to approximately 35-40 days. Based on our the vitro studies, described above the mutations of c-Met showing the greatest tumorigenic potential can be chosen and separate cell lines of H661 with the various mutations of c-Met (along with a wild type control) can be generated. Using 14 mice per group, 84 mice (assuming there will be 5 specific mutations with biological significance) can be used for this study. Measuring perpendicular tumor diameter will monitor tumor growth. Tumor volumes can be calculated using the formula π×L×D²/6 (where D=perpendicular diameter and L=longest diameter). The body weight can be measured twice a week to monitor toxicity.

### Statistical Analysis and Power Considerations

For each of the biological functional assays (cell growth and viability, cell survival, apoptosis, clonogenic survival/transformation potency) three separate experiments can be carried out with each experiment performed in triplicate, providing nine replicates per group. Each of these assays provides a quantitative outcome, which can be analyzed using analysis of variance (ANOVA) to examine the effects of c-Met mutations, HGF dose, and (when included as a third factor) the presence/absence of FCS. For a specific pairwise contrast (for example, c-Met mutation vs. control at a particular dose of HGF) nine replicates per group provide 80% power to detect a 1.4 standard deviation (SD) difference in means, using a two-sided test at the 5% significance level. This calculation assumes the between day component of variability is small or negligible.

The nude mouse studies yield longitudinal data on tumor growth (as well as body weight). Animals can be followed for 35-40 days and can have measurements performed twice a week, yielding 10-12 measurements per animal. These xenograft studies can be analyzed using a linear model with repeated measures (Crowder et al., London: Chapman and Hall. (1990))

yᵢⱼₖ=α+(β₀+βᵢ)tₖ+εᵢⱼₖ,

where yᵢⱼₖ is the tumor volume measurement for animal j in group i at time tₖ, β₀+β₁ is the time-by-treatment interaction, and εᵢⱼₖ is the random error term with covariance structure reflecting the longitudinal within-subject component. Due to the large number of repeated measurements within each mouse, this model treats time as a continuous variable. It assumes a common intercept, α, linear growth rates, and different slopes across the groups (mutant vs. control). The response variable can be appropriately transformed in order to obtain a linear time effect. If no such transformation is possible, higher order terms (quadratic or possibly cubic) can be added to capture the nonlinearity of tumor growth. Residuals can be carefully examined to verify that the model provides an adequate fit and that the covariance structure is appropriately modeled. An alternative rank-based approach by Koziol et al. (Koziol et al., Biometrics, 37, 383-390 (1981) can also be employed.

Calculations are based on 85% power, a two-sided α=0.01 to allow for multiplicity, and the ability to detect a difference in rate of change (slope) between two groups that results in a 1.5 σ (standard deviation) difference in means between the groups by 35-40 days, which would correspond to a 30% difference in the mean final tumor volume. Results indicate that, depending on the assumed correlation between serial measurements within animals (autoregressive with autocorrelation coefficient, ρ, ranging between 0.25 and 0.75), 8-14 animals per group would be sufficient to detect the above stated difference in slopes. These calculations allow for the loss of 2-3 animals per group. To ensure adequate statistical power 14 animals can be studied per group.

### Cell Scattering

One of the most important functions for c-Met has been cell scattering once stimulated with HGF. These assays are best performed in adherent cell lines since this type of assay requires attachment to the extracellular surface. Thus, the NHBE and H661 cells generated above can be used (see above) with the various c-Met constructs. The scattering assays can then be performed as previously detailed (Jeffers et al., Proc Natl Acad Sci USA, 95, (24), 14417-22 (1998)). 7.5-10×10⁴ cells/100 μl, in media (with and without FCS, and with and without HGF) can be plated in 96-well plates. Following 2 week incubation at 37° C., cells can then be stained with 0.5% crystal violet in 50% ethanol (vol/vol) for 10 min at room temperature, and scattering viewed with a light microscope and quantified.

### Example 6

### Determination of the Biological and Biochemical Effects of Specific Inhibitors Against c-Met in NSCLC Cell Lines

The NSCLC cell lines, as described above, can be used in this analysis. The effects of novel tyrosine kinase inhibitors against c-Met on HGF-mediated and serum-stimulated growth can be determined using several of the other assays described above, including those assays that measure cell growth/viability, cell survival, and apoptosis.

Small Molecule Inhibitors of c-Met

Data has already been described with regard to the 1st (SU11274) and 2nd (PHA665752) generation of c-Met small molecule inhibitors in lung cancer (Ma et al., Cancer Res, 65, (4), 1479-88 (2005); (Ma, et al., Clin Cancer Res, 11, (6), 2312-9 (2005); and Jagadeeswaran, et al., Cancer Res, 66, (1), 352-61 (2006)). Data on a 3^{rd} generation of c-Met inhibitor, Compound X is described above, and further experimentation on this inhibitor can also be deduced. Dose response (0-2 μM) and kinetics (up to 96 hours) of inhibition of various NSCLC cell lines can be performed. In addition the effects of c-Met inhibitors against the anchorage-independent growth in cell lines generated previously with c-Met mutants using a 96-well soft agar assay can also be performed as described previously Ke et al., Biotechniques, 36, (5), 826-8, 830, 832-3 (2004)).

### Antibodies

An antibody against the c-Met Sema domain (anti-Met 5D5 FAB) is currently being developed in a humanized form by Genentech for potential use in clinical trials. Anti-Met 5D5 GAB binds to the Sema domain of the Met receptor and acts as an antagonist of the Met receptor, blocking dimerization (Kong-Beltran et al., Cancer Cell, 6, (1), 75-84 (2004)). These antibodies can be particularly useful against the sema domain mutations.

### Peptide Competitive Inhibition

NK4 is a large molecule and is an antagonist of HGF, and was previously reported to be generated by proteolytic digestion of HGF. NK4 is a truncated HGF composed of the NH2-terminal hairpin domain and four kringle domains in the α-chain of HGF. It retains c-Met receptor binding properties without mediating biological responses. NK4 antagonizes HGF-induced tyrosine phosphorylation of c-Met, resulting in inhibition of HGF-induced motility and invasion of HT115 human colorectal cancer cells, as well as angiogenesis (Parr et al., Int J Cancer, 85, (4), 563-70 (2000)). NK4 can also be used to study the inhibition of NSCLC cells (Maemondo et al., Mol Ther, 5, (2), 177-85 (2002)).

Dominant Negative c-Met

Using a dominant negative c-Met, NSCLC cell lines can be transfected with the dominant negative c-Met to determine its effects. The potential inhibition of these cell lines can be determined using the strategies outlined above.

Determination of the In Vivo Effects of Inhibiting c-Met in NSCLC Xenograft Model

Nude mouse modeling can be used to determine the antitumor activity of c-Met inhibitors in NSCLC cell lines. The A549 cell line (classic for NSCLC) can be used in the xenograft model. The xenografts can be initiated by S.C. injection of 1×10⁷ cells in 0.1 or 0.2 ml of PBS into the right flanks of male nu/nu (nude) mice. Animals can be examined daily. Measuring perpendicular tumor diameter will monitor tumor growth and tumor volume can be calculated. The body weight is measured twice a week to monitor toxicity.

For studies with c-Met inhibitors, when tumor size reaches 5-7 mm, the mice can be randomized and divided into three experimental groups of 10 mice each. Each mouse can be injected intraperitoneally. The dosing can be 2 times/week. c-Met inhibitor (Compound X) is dissolved in lactate base with propylene glycol and DMSO as cosolubilizers and administered in a randomized fashion to three groups: Group 1—solvent alone for i.p., 2 X/week for 35-40 days; Group 2—c-Met inhibitor dose (50 mg/kg) for i.p., QD for 35-40 days; Group 3—cisplatin (5 mg/kg/dose, i.p. once) as positive drug control.

Determination of the Effects of Mutations and Inhibition of c-Met In Vitro (Biological and Biochemical) and In Vivo

Using the H661 cell line (containing minimal to no c-Met expression) and NHBE cells transfected with the various c-Met wild-type and mutant constructs, the biological and biochemical effects in the context of c-Met inhibitors can be systematically determined as outlined in the examples above.

### Example 7

As described above, c-Met is the receptor for hepatocyte growth factor and is a known proto-oncogene. HGF c-Met signal leads to pleiotropic cellular functions including proliferation, morphogenesis, migration, and angiogenesis (Matsumoto et al., J Biochem (Tokyo) 119, 591-600 (1996)). c-Met has vital roles during embryogenesis in differentiation and development and in adults in regeneration and repair of tissues (Huh, C. G. et al. Proc Natl Acad Sci USA 101, 4477-82 (2004) and Birchmeier et al., Trends Cell Biol., 8, 404-10 (1998)). When aberrantly expressed or activated, c-Met signaling causes cellular transformation, neoplastic invasion, and metastasis (Schmidt, L. et al. Nat Genet. 16, 68-73 (1997) and Jeffers, M. et al. Proc Natl Acad Sci USA 95, 14417-22 (1998)). Mutation causing underexpression of c-Met has also been associated with autism (Campbell et al., Proc Natl. Acad Sci USA 103, 16834-9 (2006)). In this study, the distribution and characteristics of c-Met mutations in lung cancer was studied using lung cancer cell lines and tumor tissues. There were differences in the type and frequency of mutations between Caucasians, African-Americans, and Taiwanese. N375S mutation in the HGF binding sema domain of c-Met was detected in 13.5% of Taiwanese, whereas it was completely absent in African-Americans. Also, a number of single incidence mutations were detected in the sema domain of c-Met. c-Met mutation R988C in the juxtamembrane domain was detected in two US patients but not in the Taiwanese. No tissues displayed missense mutations in the tyrosine kinase domain of c-Met. All the c-Met mutations detected in tumor tissues, for which corresponding lymphocyte or adjacent normal tissues were also available, were found to be germline. The presence of N375S mutation correlated to a higher incidence of squamous cell lung cancer in males with smoking history. In contrast, all the EGFR tyrosine kinase domain mutations were somatic and occurred more in Taiwanese samples (32%) as compared to US (3%).

### Methods

### Tissue Specimen and DNA Isolation

The tumor tissue samples were collected at the time of surgery from pathologically documented patients enrolled at The University of Chicago Hospitals, Chicago, USA for whom clinical data were available with informed consent and in conformation with institutional guidelines. The corresponding normal lung tissues obtained at surgery, where available, were also used in sequence analysis. Genomic DNA was isolated from fresh-frozen or archival formalin-fixed, paraffin-embedded tumor tissue using standard procedures. Genomic DNA was isolated from fresh frozen tumor and adjacent normal tissues and from peripheral blood of 141 Taiwanese lung cancer patients with informed consent. Genomic DNA from 74 established lung cancer cell lines was isolated by standard method.

### Nucleotide Sequence Analysis of Candidate Genes

The individual exons were amplified by multiplex PCR using QIAGEN Multiplex PCR reagent (Hilden, Germany). In each multiplex PCR reaction 5-6 sets of amplification primers were used. Only primers amplifying non-overlapping regions of genes were included in the same reaction. For DNA isolated from paraffin-embedded tissue the amplicon size was kept at <600 bp. PCR was performed in 15 μL volumes containing 1× buffer, 200 nM of each primer and 100-200 ng of template DNA. Genomic DNA obtained from fresh-frozen or of archival formalin-fixed, paraffin-embedded tumor tissue was used as template. Primer cocktails were prepared from 50 μM stock solutions. PCR profile: 95° C. for 15 min, then 35 cycles of 94° C. for 30 sec, 60° C. for 1 min, and 72° C. for 1 min, followed by one cycle of 72° C. for 10 minutes. For sequencing, PCR products were purified by adding 2 μL of Exosap to individual reactions and by incubation at 37° C. for 30 min followed by 80° C. for 15 minutes. Sequencing was performed on the forward coding strand with confirmation of c-Met alterations performed by sequencing the reverse strand as well.

Sequencing was performed on an Applied Biosystems 3730XL 96-capillary DNA Analyzer. Chromatograms were analyzed for mutations using Mutation Surveyor v2.61 (Softgenetics). The nucleotide positions numbering is relative to the first base of the translational initiation codon according to the full-length cDNA for EGFR, KRAS2 and p53. For c-Met, the cDNA of the splice variant that includes the “wild-type” full-length 154 by exon 10 was used (Ensembl peptide ID: ENSP00000317272). IDs of other CCDS used: EGFR—5514.1, KRAS2-8702.1, p53-11118.1.

### Structure Modeling

Using 1SHY chain B as template, mutations were modeled by MODELLER and SWISS-model. The non-synonymous/synonymous rate ratio (ω=d_{N}/d_{S}) was calculated by PAML package, an implementation of the maximum likelihood method for estimating w values. Homologous sequences were collected from HSSP database and PSI-Blast searching. The ω was repeatedly estimated (20 times) using different initial v value that is assigned to all amino acid sites. The initial ω values range from 0.01 to 2.00, at an interval of 0.1. Promate, an interface prediction program which combined many significant interface properties was used to predict potential protein binding sites. Discrete Optimized Protein Energy (DOPE) was used to evaluate the accuracy of models. DOPE is a distance-dependent statistical potential based on a physical reference state that accounts for the finite size and spherical shape of proteins. Root Mean Square Deviation (RMSD) values were calculated from Ca carbon of 1SHY B chain and mutation models.

### Statistical Analysis

Relationship between mutation carriers and patient clinical and behavioural characteristics were analyzed using Fisher's exact test.

### Results

Lung cancer is characterized by the accumulation of multiple genetic and/or epigenetic alterations resulting in the activation of oncogenes and the inactivation of tumor suppressor genes. Deregulation of receptor tyrosine kinase activity by somatic mutation or chromosomal alteration is common in malignancies and plays a central role in cell proliferation, metastasis, and angiogenesis (Sawyers, Genes Dev 17, 2998-3010 (2003)). Recent studies have shown that growth factor/scatter factor (HGF/SF) and its receptor c-Met play major roles in the malignant progression of numerous tumors (Birchmeier et al, Nat Rev Mol Cell Biol 4, 915-25 (2003) and Trusolino & Comoglio, Nat Rev Cancer 2, 289-300 (2002)). Even though amplification and activating mutations have been said to be the major contributors of c-Met receptor tyrosine kinase activation in several cancers, the nature and distribution of mutations as well as their functional significance is not completely understood. In this study, lung tissue from Taiwanese, African-American, and Caucasian lung cancer patients was screened (n=283) for c-Met and EGFR mutations, and those with mutations were further screened for KRAS2, and p53 mutations. Seventy-four lung cancer cell lines were similarly screened. The relationship between mutation profiles and patient ethnicity, behavioral factors, and clinicopathologic characteristics were evaluated.

Sema (extracellular), juxtamembrane and tyrosine kinase (both intracellular) are the important functional domains of c-Met (FIG. 2). The sema domain (amino acids 25-519) has sequences involved in ligand binding, dimerization, and heparin binding. The juxtamembrane domain (JM) (amino acids 956-1093) contains negative regulatory protein (c-Cbl) binding regions, and the tyrosine kinase (TK) domain of c-Met is responsible for ATP binding and transphosphorylation.

Individual exons of sema, JM, and TK domains of c-Met were amplified by multiplex PCR and mutations identified through sequencing. Five out of six mutations detected in c-Met were in the sema domain (FIGS. 2 and 3a). A G1124A substitution, leading to amino acid change N375S, occurred in 13.5% of Taiwanese tumor tissues (n=141), one was homozygous (FIG. 5), but in only 2.6% of Caucasian tumor tissues (n=76) and in none of the African American tumor tissues (n=66) and African American derived cell lines (n=8). The N375S allele was also detected in all corresponding adjacent normal and lymphocyte DNA tested, which confirmed that it was a germline mutation. The high frequency of N375S mutation in Taiwanese and its complete absence in African Americans signifies the need to consider race as an important factor in c-Met mutational studies. N375S was detected in 2 SCLC(H289 and HCC33) and an adeno (H2122) cell lines all of which were derived from Caucasian (FIG. 5). A C632G alteration resulting in L211W amino acid change occurred in a Taiwanese tumor tissue, its adjacent normal tissue, and the corresponding lymphocyte DNA. Among US tumor tissues, additional sema domain mutations resulting in amino acid changes A347T, E355K, and M362T were found, of which E355K was also detected in the adjacent normal tissue. Adjacent normal tissues were not available for A347T and M362T mutation carriers. All the missense mutations detected in the sema domain were non-repeating, except for N375S.

The sema domain of c-Met contained silent mutations C534T and C1131T at amino acid residues 5178 and 1377, respectively, in a number of tissues (FIG. 3a and FIG. 5). C534T was linked to G1124A (missense mutation N375S) in all Taiwanese and US Caucasian tumor tissue, adjacent normal and lymphocyte DNAs. Taiwanese tissue #41 was homozygous for both these mutations. But, C534T was detected in isolation in African American tumor tissues. Silent mutation C1131T occurred in a large percentage of African American and but was not found in Taiwanese.

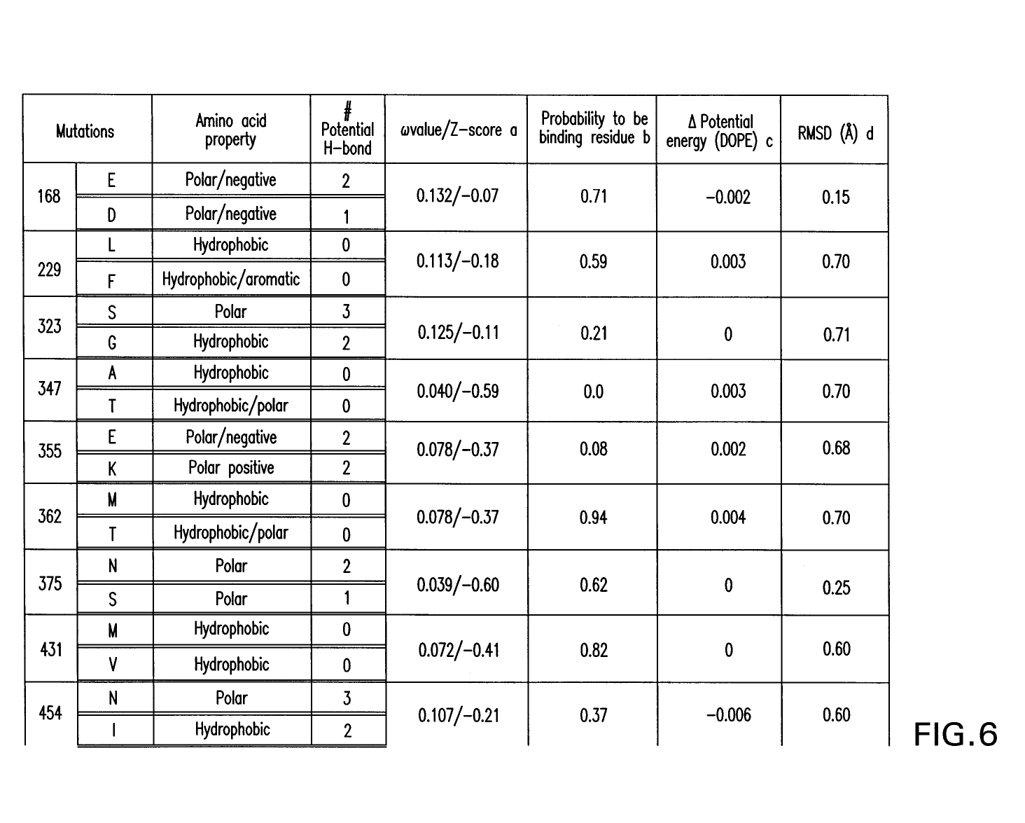

The sema domain has been shown to be necessary and sufficient for c-Met binding to its ligand, HGF, and to heparin and subsequently promote receptor dimerization and activation (Zhang et al., Cancer Cell 6, 5-6 (2004)). Therefore, alterations in the primary sequence can be expected to affect the functionality. The crystal structure of the sema domain in complex with β chain of HGF has been determined recently (Stamos et al., Embo J 23, 2325-35 (2004)), providing the opportunity to study the potential effects of several of these mutations on ligand binding through comparative modeling. Analysis of the 3D structure of sema of c-Met-HGF complex (1SHY) showed the presence of sema domain residues E168 and L229 in direct contact with HGF, indicating the potential for altering the binding affinity of the complex due to changes in these residues (FIG. 3bi). Also, the 3D model showed residue E168 to be in a conserved motif due to the high ω value (>1) of the neighboring residues P169, S170 and P210 (FIG. 3bii). Replacement of asparagine at 375 by a serine resulted in the loss of a hydrogen bond between the mutant and the adjacent arginine residue at 280 in the 3D structure (FIG. 3biii). Overall, mutations of sema domain only subtly change the protein's physical properties, as the Root Mean Square Deviation (RMSD) values were all less than 1 Å (FIG. 6).

However, the mutations still share some common trends, such as loss of hydrogen bonds and, in a majority of cases, increased potential energy. Since both the HGF/c-Met complex and the dimerized form of the receptor have only weak interactions, subtle mutations may be enough to affect protein functions.

Cloning and expression of c-Met mutants E168D and L229F in Cos7 cells followed by immunoblotting following protocols described previously (See Ma et al., Cancer Res 63, 6272-81 (2003) and Ma et al., Cancer Res 65, 1479-88 (2005)) showed prolonged activation of the downstream phosphorylation of both p-c-Met and p-AKT upon HGF stimulation compared to wild-type c-Met (FIG. 3c).

A c-Met genotype-phenotype frequency analyses was also conducted. The distribution of c-Met mutations in lung cancer tissues based on ethnic groups and histological subtypes was carried out. In Taiwanese, frequency of N375S c-Met mutations strongly correlated with the incidence of squamous cell carcinoma (P=0.051). Additionally, gender may be significant factor in predisposing c-Met mutation carriers to lung cancer as seen from the higher frequency of N375S mutations in males (P=0.043). A large percentage of N375S c-Met mutation carriers were smokers. Squamous cancer occurred much later in the lifetime of these patients (Median age, 73 years) (FIG. 5). Additionally, all the Taiwanese lung cancer patients who were non-smokers (n=21) had only the adeno histological sub-type.

The JM domain mutation C2962T leading to amino acid change R988C was detected in two tumors from patients and corresponding adjacent normal tissues (FIG. 3a). This previously reported JM domain mutation was absent in Taiwanese. The JM domain negatively regulates c-Met signaling by targeting c-Met for lysosomal degradation via c-Cbl ubiquitin ligase mediated ubiquitination. The JM domain mutation R988C was shown to have an overall positive gain-of-function effect on various parameters, such as transient growth factor-independent proliferation, tumorigenicity, and altered c-Met signaling with enhanced tyrosine phosphorylation, as well as enhanced cell motility and migration. The SWR/J mouse strain with a R968c c-Met variant mutation (human analogue of R988C) has been shown to predispose these mice to higher incidence of lung cancer (Zaffaroni et al., Oncogene 24, 1084-90 (2005)). When the entire stretch of c-Met coding region (exons 2-21) was sequenced in 74 cell lines, a homozygous mutation resulting in amino acid change 1852F in the IPT-4 domain was detected in NSCLC cell line H157.

The aberrant activation of EGFR caused by activating mutations results in enhanced cell proliferation and other tumor promoting activities. The EGFR TK domain, encoded by exons 18-21, has been shown to be the site of all activating mutations in NSCLC. Unlike the c-Met missense mutations in this study, none of the EGFR mutations detected in lung cancer tissues were present in the adjacent normal or corresponding lymphocyte tissues, indicating that all EGFR alterations were true somatic mutations (see Table 11).

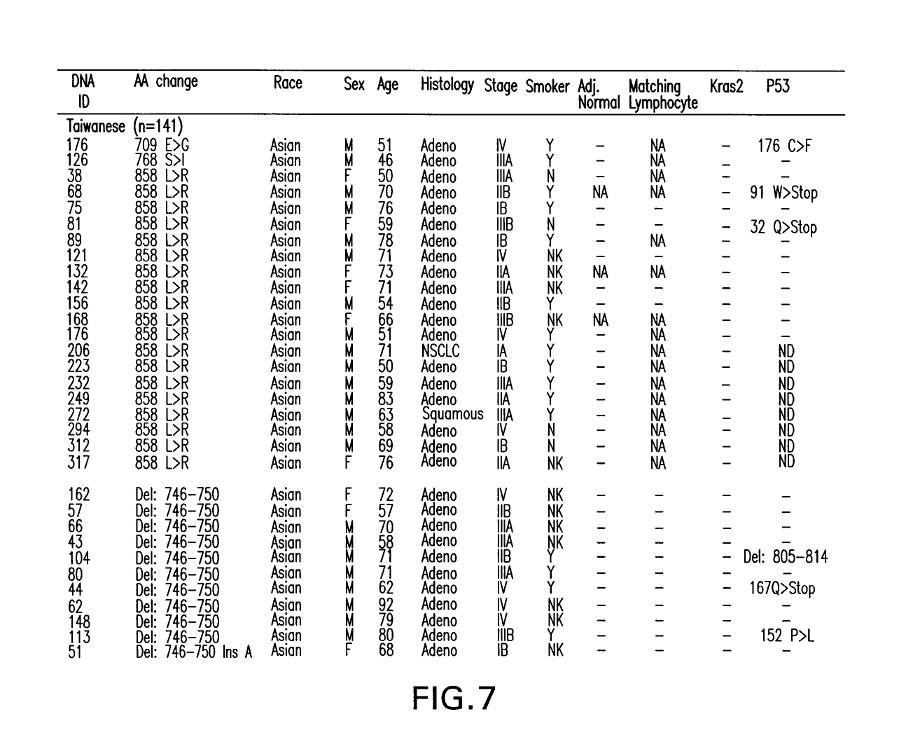

Taiwanese and US patients had different EGFR mutation profiles (FIG. 7). Mutations in the EGFR TK domain were detected in 32% Taiwanese patients (n=141), compared to only 3% (n=142) of US patients. Histologically, among Taiwanese patients, 96% of the EGFR TK domain mutations were found in adenocarcinoma, and 4% in squamous cell carcinoma, whereas large cell (n=7) and SCLC (n=2) subtypes showed no mutations. Point mutations resulting in amino acid changes and in-frame deletion mutations were detected in similar percentages of tumor tissues. The predominant point mutation L858R in Taiwanese was absent in US patients. Overall, results of EGFR mutation characteristics among Taiwanese lung cancer patients were similar to earlier reports on East Asian patients.

TABLE 11
Summary of EGFR tyrosine kinase domain missense mutations
in lung cancer tissues of different ethnic groups

| | | | | | Smoking | Smoking | | |
|---|---|---|---|---|---|---|---|---|
| Ethnic | Mutation | Deletion | Histological | | History | History | Gender | Gender |
| Group | Carriers | Mutation | Subtype | | Y | N | M | F |
| Taiwanese | 32 | 53 | 96 (91) | adeno | 24 (83) | 39 (23) | 31 (106) | 34 (35) |
| (n = 141) | | | 4 (37) | squamous | | | | |
| Caucasian | 1.4 | 0 | 50 (28) | adeno | 3 (69) | 0 (2) | 2 (51) | 4.8 (21) |
| (n = 76) | | | 50 (12) | large cell | | | | |
| African- | 1.4 | 50 | 100 (15) | adeno | 5.6 (36) | 0 (3) | 6.3 (32) | 0 (34) |
| American | | | | | | | | |
| (n = 66) | | | | | | | | |

Out of 283 lung cancer tissues screened, a Taiwanese patient with adenocarcinoma had both c-Met (N375S) and EGFR (homozygous deletion 746-750) mutations, and one US large cell carcinoma patient had both c-Met (R988C) and EGFR(P848L) mutations (FIG. 5).

Screening c-Met mutation carriers for KRAS2 exon 1 (codons 12) mutations revealed 3 patients with mutations in both. None of the tumor tissues of Taiwanese or US had both EGFR and KRAS2 mutations. c-Met or EGFR mutation carriers in a batch of Taiwanese patients (n=71) were also screened for p53 mutations by amplification and sequencing of all coding exons. One patient with a KRAS2 (G12A) mutation had a p53 mutation along with a N375S c-Met mutation (FIG. 5). p53 mutations were found in 18% (n=11) of c-Met and 29% (n=29) of EGFR mutation carriers, respectively (FIG. 5 and Table 11). p53 mutations in the Taiwanese tumor tissues showed no significant trends.

In summary, in lung cancer patients, missense mutations in the sema and JM domains of c-Met were germline and not somatic whereas all EGFR TK domain mutations were somatic, c-Met TK domain mutations were absent, Taiwanese and AA had different c-Met genotypes and relatively more frequent and non-repetiting mutations occurred in sema domain of c-Met.

### Example 8

### c-Met Expression and Amplification in SCCHN Tumor Tissues and Cell Lines

In order to understand the role of c-Met in SCCHN, c-Met expression was examined in a large array of paraffin embedded tissue micro arrays (TMA) of SCCHN tumor and normal samples. Tumor Tissue Micro Array (TMA) analysis of N=121 tissue samples by IHC (3D4 Zymed/Invitrogen mouse monoclonal AB was performed. There was increasing over-expression in the transition from normal tissue (21%), via dysplasia (30%) to frank SCCHN (84%). Many of the normal tissue samples were from “macroscopically normal” mucosa adjacent to tumor and may therefore not be normal at the molecular level, which may explain the low degree of moderately high expression in normal mucosa. Immunoblot for protein expression from lysates of the SCCHN cell lines using antibodies against c-Met and β-Actin, the later as a loading control. FISH analysis for amplification of c-Met and EGFR/or centromeric protein (Control) in SCCHN cell lines were also performed. The expression of the c-Met protein in various SCCHN cell lines was examined using standard immunoblotting with the antihuman monoclonal c-Met antibody (3D4, Zymed/Invitrogen). It was found that there was high expression of c-Met in most of the SCCHN cell lines (86%), including SCC9, SCC15, SQ-20B, SCC25, SCC68, MSK921, JSQ3 and HN31. Moderate expression was seen in SCC61, and low expression in SCC17B. Twelve head and neck squamous cell carcinoma cell lines were also analyzed for amplification of the c-Met region using Fluorescent in situ hybridization (FISH) technique. Previously, Smolen et al. reported c-Met amplification in gastric cancer (Smolen et al., Proc Natl Acad Sci USA 103, (7), 2316-21 (2006), and correlated amplification with sensitivity to c-Met inhibition. The results obtained showed c-Met amplifications in head and neck cancer and the second tumor type after gastric cancer. Analysis of 12 cell lines further revealed amplification of the c-Met region on chromosome 7p11.2 in 3 cell lines (30%).

### Example 9

### c-Met Mutations in SCCHN Tumor Tissues and Cell Lines

Fifty-nine SCCHN tumor tissues and 8 SCCHN cell lines were sequenced for mutations in the c-met gene. The entire gene was analyzed. Results from 52 primary tumor samples and 8 cell lines revealed missense mutations in the SEMA and juxtamembrane (JM) domains in 12% of samples. In contrast to the reported literature 23, 26 (primarily TK domain sequencing) no mutations were identified in the Tyrosine Kinase (TK) domain. Subsequent analysis of metastatic SCCHN deposits (distant and lymph node metastases) in 7 samples revealed 2 tyrosine kinase mutations (TK).

In order to determine the functions of the various c-Met mutations, plasmid constructs were generated for all of the SEMA domain mutated-Met. Positive clones were selected for stable G418-resistant transfected clones for these mutated-Met variants. In addition, TK domain mutation carrying plasmids can also be produced.

### Example 10

### Inhibition of c-Met Protein Expression and Cell Viability Via siRNA Gene Silencing

To determine the effectiveness of siRNA against c-Met in SCCHN, two SCCHN cells (SCC61 and SQ-20B) were transfected with control siRNA or with c-Met specific siRNA duplexes. Cell viability was measured in triplicates by MTT assay method after 72 hr of siRNA transfection. Percentage of cell viability inhibition by c-Met targeting siRNA was shown with control siRNA in SCC61 and SQ-20B cells. Immunoblot analysis showed inhibition of c-Met protein expression by c-Met specific siRNA in SCC 61 and SQ-20B cells.

c-Met Specific Inhibition In-Vitro—Effects on Signaling, Viability and Colony Formation

Specific inhibition of c-Met by small molecule inhibitors can also be determined. For example, several ATP-competitive small molecule inhibitors of c-Met, namely SU11274 (1^{st} generation), PHA665752 (2^{nd} generation) and Compound X (3^{rd} generation) can be used for studies of c-Met inhibitory strategies. Both SU11274 (1^{st} generation) and Compound X (3^{rd} generation) were used to test for their respective inhibitory effects on SCCHN cell lines. The c-Met selective inhibitor SU11274 markedly decreased viability/growth in several cell lines measured by either MTT and/or Cyquant.

c-Met and Cell Motility

c-Met is known to have a significant role in cell scattering and possibly metastatic spread (Ren et al., Clin Cancer Res, 11, (17), 6190-7 (2005) and Endo, et al., Hum Pathol, 37, (8), 1111-6 (2006)). Here, the effects of c-Met suppression on cell motility and migration as a surrogate marker for metastatic spread was investigated. After treatment with c-Met inhibitor SU11274, a marked decrease in cell motility was noted. Marked inhibition of the c-Met phosphorylation was also observed with SU11274/Compound X in all cell lines tested, which completely reversed the effects of HGF stimulation and also affected downstream phosphorylation of SHP2, AKT, and to a lesser degree ERK. The degree of downstream modulation varied by cell line.

### Inhibition of C-Met Phosphorylation and Colony Formation of SCCHN Cells Using Small Molecule Inhibitor Compound X

The growth inhibitory effect of c-Met inhibitor Compound X in an anchorage independent manner was also analyzed using soft agar colony formation assay. Again the degree of growth inhibition with Compound X depended on the respective cell line. Colony formation assays were performed and the results for each of the cell lines tested were observed. The results showed that there was a marked reduction of in the number of colonies formed, as counted by automated image analysis. Results further revealed that constitutively activated/or amplified c-Met SCCHN cell lines such as SCC61 and SQ20 B were developing more and large size colonies in an anchorage independent manner on the soft agar plates and it was completely abrogated with 500 nM of Compound X.

### In-Vivo Inhibition of Cell Proliferation and Blood Vessel Density of SCCHN Xenograft by Compound X

In vivo c-Met inhibition was also studied. SCCHN(OSCC-3) cells were used in xenograft model and animals were treated with vehicle or Compound X (25 mg/kg b.w) p.o daily, after growth of tumors. Tumor sections from mice in control and treatment groups were examined using an antibody specific for nuclear antigen Ki-67, a marker of active cell division, and an antibody against CD31 for the detection of intratumoral microvessel density. H&E and immunohistochemical analysis of OSCC-3 xenograft tumors revealed that Compound X markedly reduced both cell proliferation (Ki67) and blood vessel density (CD31).

Soluble c-Met Levels—Pre- and Post Treatment with Compound X

In order to bring c-Met inhibition to clinical fruition, biomarkers are necessary. It was determined from the animal models the levels of a cleaved form of c-Met—called soluble Met (sMet). Serum levels of soluble c-MET with Compound X treatment in nude mice xenograft model were examined. sMET was measured by ELISA. In the mouse xenograft models, s-Met correlated with exposure to Compound X. The studies revealed a decrease in plasma levels (p<0.01) in animal xenograft models treated with c-MET inhibition.

### Example 11

The frequency of known c-Met mutations, overexpression, and amplification of c-Met in primary SCCHN versus lymph node metastases and correlation with clinical outcome and demographic factors (including tobacco and alcohol use, race, and gender) as well as determination of the prognostic implications of circulating biomarkers, sMet and HGF, in such patients can be determined.

The differences and distribution of c-Met mutations, amplification, and overexpression between primary SCCHN and matched lymph node metastases can be determined as well as being correlated with a) clinical outcome (survival), b) tobacco and/or alcohol use, and c) demographic factors including gender and race (with respect to frequencies and levels found in both tumor and normal tissue). In addition, circulating c-Met (sMet) and HGF levels in previously banked serum samples can be determined and correlated with survival. Subgroup analyses for the above mentioned demographic/clinical factors can also be determined.

c-Met is a unique receptor tyrosine kinase that can be overexpressed, activated through HGF, amplified, or mutated in SCCHN. SCCHN is characterized by early metastasis to lymph nodes. c-Met is important in the metastasis of SCCHN, and thus determination of the expression, amplification, and mutations of c-Met in SCCHN and lymph node metastases can be determined. Circulating c-Met and HGF in previously stored serum samples from SCCHN subjects can also be determined and compared with appropriate matched normal subjects.

### Mutational Analysis

Mutational analysis can be performed in the primary tumor tissue, and in the involved lymph nodes. Description of these mutations can provide further insight as to what specific mutations are causing significant phenotypic changes. The analysis utilizes 200 paired samples (primary and lymph nodes, as well normal adjacent tissue). Based on power considerations for the survival analyses, 200 cases can be used from among patients diagnosed in 2005 or earlier (see below).

### Statistical Analysis and Power Considerations

The number of mutations obtained in the primary tumor, adjacent normal tissue, and metastatic lymph nodes can also be determined using the Wilcoxon signed-rank test. It can be assumed that the number of mutations at each location will range from 0-3 with the majority being 0 even in the primary tumors. A total of 200 samples can be analyzed, which will provide 85% power to detect a mean difference of 0.36 between the primary and lymph node sites (the main comparison of interest), assuming a standard deviation of 1.5 (range of differences/4). Comparison of primary tumor with adjacent normal tissue can also similarly be performed. Two-sided tests at the alpha=0.025 significance level can be performed to allow for the two pairwise comparisons (Bonferroni adjustment).

### Example 12

### c-Met Expression Levels

In addition to mutational analysis, protein expression levels can be determined by immunohistochemical analysis in the tumor microarray. Total c-Met expression as well as phosphorylated c-Met expression can be evaluated. Tissue arrays using an automated tissue arrayer (Beecher Instruments ATA-27, Sun Prairie, USA) with 25 patient samples per block can be used to perform the analysis. The size of each core can be 1 mm. One array can contain 25 cases and 8 arrays (total of 200 cases) can be produced overall. In addition to primary SCCHN tumors, all cases included in the analysis will have corresponding metastatic lymph node samples. All cases have associated survival data/clinical information and the patient population includes 20% AA. The following data elements can be incorporated into a database for analysis: survival time (or date last known alive), race, gender, smoking and alcohol status, performance status, treatments received and comorbid illnesses.

IHC staining can be performed using biotin-free HRP-labeled polymer complex bound to secondary antibody (DAKO Cytomation, Carpinteria, Calif.). Tumor tissue arrays can be set up to enhance the efficiency of staining. Paraffin sections can be deparaffinized in xylene, rehydrated through graded ethanol solutions to distilled water and then washed in Tris-buffered saline (TBS). Antigen unmasking can be carried out by heating sections in citrate buffer (pH=6) for 15 minutes in a microwave. Endogenous peroxidase activity can then be quenched by incubation in 3% hydrogen peroxide for 5 minutes to block endogenous peroxidase activity, followed by incubation for 20 minutes in a protein blocking solution (DAKO) to reduce non-specific background. The primary antibody can be applied at room temperature for 1 hour. Primary antibodies can include anti-c-Met (Invitrogen/Zymed mouse monoclonal 3D4), anti-phospho-Met ([pY1003], BioSource International, Camarillo, Calif.), anti-phospho-Met ([pY1230/1234/1235] BioSource International), and anti-HGF (rabbit polyclonal SantaCruz Biotechnology, Calif.). This step can be followed by a 30 minute incubation at room temperature with goat anti-mouse/rabbit IgG conjugated to a horseradish peroxidase (HRP)-labeled polymer (DAKO Envision TM+ System, DAKO Corp., Carpinteria, Calif.). Slides will then be developed for 5 min with 3-3′-diaminobenzidine (DAB) chromogen, counterstained with hematoxylin, and coverslipped.

Negative controls can be performed by substituting the primary antibody step with non-immune mouse immunoglobulins. Results can then be assessed qualitatively by light microscopy: Immunohistochemistry from tumor and adjacent normal tissue can be compared and grading can be for negative (0), weak (1+), strong (2+), and very strong expression (3+).

### Statistical Analysis and Power Considerations

Comparison of the frequency of staining grades between sites can also be performed. Comparisons can be made between primary tumor center and adjacent normal tissue, the primary tumor center and invasive tumor edge, and the primary tumor center and metastatic lymph node. To control the type I error rate, these two tests can be performed at the alpha=0.017 significance level. Two sets of analyses can be conducted, one treating the expression level as an ordinal variable and the other dichotomizing the results as 0/1+ (none or weak) vs. 2+/3+ (strong) staining. Analysis on the ordinal scale can use the Wilcoxon signed-rank test; McNemar's test can be used for the dichotomized data. For the main comparison of interest, i.e., primary tumor center vs. lymph nodes, our sample size (n=200) can provide an 85% power to detect mean differences of 0.38 on the 3-point ordinal scale and 89% power to detect an odds ratio of 2.0 under McNemar's test.

Determination of Prognostic Implications of c-Met Expression, c-Met Mutations, and c-Met Amplification on Patient Survival

Survival analysis, both overall and after stratifying patients by gender, race, and smoking/alcohol status can also be determined. The purpose of these analyses is to determine whether c-Met has prognostic value and whether the effects of c-Met on outcome are consistent across the various demographic/clinical subgroups. In order to generate the required data, the association between the various c-Met parameters and race, gender, stage, and smoking and alcohol status using chisquare or nonparametric can also be performed. Rank-based tests can also be used as appropriate. Kaplan-Meier survival curves can be plotted by c-Met staining score, c-Met mutational status, and the presence or absence of gene amplification and compared using a logrank test. Univariate and multivariable Cox regression models can then be fit incorporating the c-Met staining score, mutational status, gene amplification status, age, race, gender, HPV status, smoking and alcohol history, performance status, treatment received, and the presence of comorbid conditions as potential covariates. c-Met parameters in both the primary and lymph nodes can also be evaluated. The goodness-of-fit of the proportional hazards assumption and the functional form for covariates included in the Cox regression model can be checked using graphical methods (deviance and martingale residual plots).

The power to detect the effect of a prognostic variable in a Cox regression model is dependent upon the number of deaths observed as well as the relative proportions of patients in the groups being compared. 200 cases can be chosen from among those diagnosed in 2005 or earlier.

### Example 13

### Determination of the Biological Effects of c-Met Mutations on Various SCCHN Cells

As described elsewhere herein, several novel mutations and alterations of c-Met in SCCHN have been identified. Several constructs using IRES2-EGFP have been generated with the pAcGFP1—N1 and pDsRed-Monomer-N1 vectors (Clontech) being modified into Gateway destination vectors (Invitrogen) for high expression in mammalian cells. These vectors have the advantage of having monomer fluorescence proteins as well as easily transfectable selectivity with G418. The efficiency of transfections was 80-90%. Full length wild-type c-Met as well as mutations of c-Met E168D, T230M, N375S, R988C, T1010I, Y1230C Y1253D, T1275I V1333I and other mutations, can be generated. Other constructs comprising the JM domain alone, the M1268T mutant of c-Met (as controls), the Tpr-Met fusion, can be generated and used to transfect cells. The control M1268T mutant has been well characterized in sporadic renal papillary cancer with strong auto-phosphorylation and biological activity (Miller et al., Proteins, 44, (1), 32-43 (2001)). In addition, Tpr-Met has also been shown to have potent tyrosine kinase activity and enhanced biological activity and will serve as a control. Site-directed mutagenesis using the QuickChange XL site-directed mutagenesis kit from Stratagene can also be used.

The cell lines used for the transfection can be SCC 17B cells. The SCC 17B SCCHN cell line has low detectable expression of c-Met and this would serve as an excellent model for studying the behavior of wild-type and mutant c-Met in the context of SCCHN. As described above, transfection of one of the vectors alone, yielded an efficiency rate (with lipofectamine) of approximately 60%.

Both transient transfectants and stable transfectants can be generated through G418 selection. Parental cell lines as well as vector alone transfected cell lines can be used as controls. Clonal populations of low, medium, and high expressors of the c-Met constructs can also be generated to determine the effect of expression on the various biological/biochemical functions proposed below.

### Cell Growth and Viability

Cell growth and viability of the cell lines generated with trypan blue exclusion and MTS assay can also be performed. An adapted dye conversion assay (MTS) can be used to study the various cell lines and can be utilized in the assay to study the kinetics (over 72 hours) and dose-response effects of HGF (0-100 ng/ml). Assays with and without FCS can be used to determine the effect of serum-stimulated growth. As an alternative strategy (also for many of the assays below), HGF can be transfected concomitantly with the c-Met constructs to determine the effects of HGF endogenously.

### Cell Survival and Apoptosis

Cell survival assays can be performed for the cell lines generated above. The cell survival of SCC 17B SCCHN cells with the various transfected c-Met constructs can be determined, since this will closely reflect the SCCHN cancer behavior. Initially, cells can be serum deprived and a survival curve with and without HGF can be generated (a concentration of 100 ng/ml has been determined to be reasonable for survival in SCCHN cell lines). This can be determined via trypan blue exclusion assay as described above.

The ability of the transfected cells to undergo (or have reduced) apoptosis (with and without HGF) can also be determined. SCC 17B cell lines generated as above will be used and apoptosis can be determined as described for H661 cells (see earlier).

### Cell Scattering

One of the most important functions for c-Met has been cell scattering once stimulated with HGF. These assays can be performed in adherent cell lines since this type of assay requires attachment to the extracellular surface. Thus, SCCHN cells generated above can be used with the various c-Met constructs. Scattering assays can then be performed as previously detailed (Jeffers et al., Proc Natl Acad Sci USA, 95, (24), 14417-22 (1998)). 7.5-10×10⁴ cells/100 μL in media (with and without FCS, and with and without HGF) can then be plated in 96-well plates. Following 2 week incubation at 37° C., cells are then stained with 0.5% crystal violet in 50% ethanol (vol/vol) for 10 min at room temperature, and scattering viewed with a light microscope and quantified.

### Transformation

Clonogenic assays can also be used to determine cell survival/transformation potency of SCCHN transfected with various c-Met constructs. SCCHN cells such as SCC 17B are able to form colonies in an agarose medium. Soft agarose assays can also be employed for transformation ability in the various cell lines generated.

### Nude Mouse Modeling

Using transfected SCC 17B cell lines with c-Met constructs, the tumor activity in a nude mouse model can be determined as previously described (Andoniou et al., Oncogene, 12, (9), 1981-9 (1996)) and detailed above.

### Tumor Growth Characteristics of Compound X Treatment Model

The OSCC-3 cell lines can be used to determine the tumor activity in a nude mouse model. The xenografts can be initiated by S.C. injection of 1×10⁷ cells into the right flanks of male (nude) mice. Animals can be examined daily. Measuring perpendicular tumor diameter will monitor tumor growth and tumor volume can be calculated. The body weight is measured twice a week to monitor toxicity. For studies with c-Met inhibitors, when tumor size reaches 5-7 mm, the mice are to be randomized and divided into three experimental groups of 10 mice each. c-Met inhibitor (Compound X) is administered by oral route using animal feeding needles (Popper and sons, inc.(new hydepark, NY). c-Met inhibitor (Compound X) and its solvent administered in a randomized fashion to three groups: Group 1—solvent alone p.o., daily for 35-40 days; Group 2—c-Met inhibitor (25 to 50 mg/kg) p.o., for 35-40 days; Group 3—cisplatin (5 mg/kg/dose, i.p. once) as positive drug control.

Tumor Growth Characteristics of c-Met Mutants In Vivo

Using the SCC 17B cell line (containing low endogenous c-Met expression) transfected with the various c-Met wild-type and mutant constructs, the biological and biochemical effects in the context of c-Met inhibitors in-vitro as described above can be determined. From these in vitro studies, the mutations exhibiting the greatest degree of tumorigenicity are selected, and in vivo mouse studies performed using the corresponding cell lines for xenografting. For each mutant construct, mice are randomized to groups 1, 2, and 3 as described above (solvent alone, c-Met inhibitor, and cisplatin) and followed for 35-40 days.

### Example 14

As described above, a number of germline mutations in c-Met were identified in solid tumors. To investigate the in vivo function and molecular mechanism of mutations in the c-met gene in the juxtamembrane domain in relation to lung cancer, transgenic Caenorhabditis elegans strains were generated that harbour wild type or mutant human R988C and mutant T1010I. The evidence described below provides that c-Met mutations can be studied in C. elegans, and that carcinogens can enhance c-Met or mutant function, thereby ultimately leading to lung cancer.

### Maintenance of the Nematode Strains and Culture Conditions

Wild type C. elegans N2 strains were obtained from the Caenorhabditis Genetics Center at the University of Minnesota, St. Paul, Minn.; grown on standard nematode growth medium (NGM) seeded with E. coli strain OP50 as a food source at 20° C. as described by Brenner et al. (Brenner, Genetics 77, 71-94 (1974).

Human c-Met Constructs, Site Directed Mutagenesis and Cloning.

The coding sequence of c-Met was PCR amplified using and inserted into pENTR/D-Topo vector using primers 5′-CACCATGAAGGCCCCCGCTGTGCTTGC-3′ (SEQ ID NO: 151) and 5′-TGATGTCTCCCAGAAGGAGGCTGGTCGTGTG-3′ (SEQ ID NO: 152). Wild-type c-Met was cloned into a pIRES2-EGFP bicistronic vector (Ma, et al., Cancer Res 65, 1479-88 (2005)). c-Met from pENTR/D-TOPO vector was subcloned into the expression vector pDEST47 through an LR recombination reaction (Invitrogen, CA). Mutants of c-Met were created using a Quickchange Site Directed Mutagenesis kit (Stratagene, Calif.) using the following pairs of primers: 5′-GATCTGGGCAGTGAATTAGTTTGCTACGATGCAAGAGTACAC-3′ (SEQ ID NO: 153), 5′-GTGTACTCTTGCATCGTAGCAAACTAATTCACTGCCCAGATC-3′ (SEQ ID NO: 154) and 5′-CAAGTGCAGTATCCTCTGACAGACATGTCCCCCATCCTAAC-3′ (SEQ ID NO: 155), 5′-GTTAGGATGGGGGACATGTCTGTCAGAGGATACTGCACTTG-3′ (SEQ ID NO: 156) for R988C and T1010I, respectively. The constructs for R988C and T1010I were created using a Quickchange Site Directed Mutagenesis kit (Stratagene, Calif.). The mutant c-Met cDNA for the R988C mutation, occurring in the juxtamembrane domain of c-Met changes an arginine residue into a cystine residue at position 988, whereas the mutant c-Met cDNA for the T1010I mutation changes the threonine residue into an isoleucine at the residue 1010.

PCR Amplification of Human c-Met in Transgenic and Control C. elegans Lines

The genomic DNA was extracted from single wild type and single transgenic worms, as described by Barstead et al. (Barstead et al., Cell Motil Cytoskeleton, 20(1):69-78 (1991)), and amplified the human c-Met cDNA by using specific primers 5′-ATGAAGGCCCCCGCTGTGCTTGC-3′ (SEQ ID NO: 157) and 5′-TGATGTCTCCCAGAAGGAGGCTGGTCGTGTG-3′ (SEQ ID NO: 158) to confirm the expression of human c-Met in transgenic lines. Single animals were picked up with a platinum wire and each placed in a 2.5 μl drop of lysis buffer (60 μg/ml proteinase K in 10 mM Tris (pH 8.2), 50 mM KCL, 2.5 mM MgCl₂, 0.45% Tween 20 and 0.05% gelatin) in the cap of a separate 0.5 ml tube suitable for PCR. The drops were then moved to the bottom of the tubes by a brief microfuge spin, frozen (−70° C., 15 min), and after the addition of a mineral oil overlay, heated (60° C., 1 hr followed by 95° C., 15 min). After cooling to 4° C., 22.5 μl of a master mix were pipetted on top of the mineral oil overlay. The mix is formulated to bring the reaction volume to 25 μl with these final conditions: 25 pmol of primers, 10 mM Tris (pH 8.3), 50 mM KCL, 1.5 mM MgCl₂, 0.001% (w/v) gelatin, 0.2 mM dNTP, and 0.6 unit Tag polymerase (Invitrogen). A brief microfuge spin was used to move the mastermix through the mineral oil overlay, and the reactions were rapidly heated (within 1 min) to 94° C. and cycled 30 times: 94° C. for 30 s, 58° C. for 1 min, and 72° C. for 1 min. Gel lanes were loaded with 5W of each reaction.

Western Blot Analysis of c-Met Protein Expression in Wild Type and Mutant c-Met Transgenic C. elegans

The wild type and transgenic animals were collected directly from NGM plate, washed with M9 buffer three times. The lysates were clarified by centrifugation at 15000 g for 10 min at 4°, and the resulting supernatant was used for the protein analysis. After determination of total protein (Bradford method), the samples were boiled for 5 min in the presence of sodium dodecyl sulphate-polyacrylamide gel electrophoresis (SDS-PAGE) loading buffer and then loaded onto the gel. Proteins were separated on precast 4-12% gradient gels (Invitrogen) and transferred onto PVDF (polyvinylidene difluoride) membrane.

The membrane was then incubated with a solution containing Tris-buffered saline, 0.05% Tween 20, and 5% (w/v) non-fat dry milk and then exposed to the c-Met rabbit polyclonal primary antibody (Santa Cruz Biotech.) at a dilution of 1:1000 for overnight at 4°. The bands were visualized using enhanced chemiluminescence's method following treatment with the goat anti-rabbit IgG conjugated with HRP, secondary antibody (at a dilution of 1:4000). The blots were stripped and re-probed with anti-β actin antibody as a control for protein loading. Immunoblotting for each protein was performed at least twice using independently prepared lysates.

### Statistical Analysis

The statistical package SPSS 15.0 (Chicago, Ill.) was used for all statistical calculations. When comparing incidence of phenotypes, Fisher's exact test was used to estimate significance. Each group of animals had 300 worms.

C. elegans Transgenic Lines, Microinjection of c-Met cDNA Constructs and Screening Phenotypes

To examine the effect of human c-met gene expression in C. elegans, both wild-type c-Met cDNA and mutant c-Met cDNA were individually micro-injected into young adult wild type N2 hermaphrodite gonads (gonad syncitium), at a concentration of 25 μg/ml. The constructs for the mutant c-Met cDNA were generated as described above. Control animals (N2) were injected with vector alone. Injected animals were individually cloned and their progeny were observed under dissecting microscope. Progenies were isolated and examined for visible morphological abnormalities such as defects in vulval formation, body morphology and locomotion. Individually cloned transgenic animals were further analyzed for fecundity and viability.

Animals in the progeny harboring a visible phenotype such as vulva defect, body morphology change, locomotion defect, and paralysis; and animals showing hyperactivity were isolated, individually cloned and examined with dissecting microscope. For detailed analysis of the phenotype, individual animals were mounted on an agar pad and observed under a Nikon Eclipse E600 fluorescence microscope equipped for Nomarski optics. Images were taken by a Hamamatsu Photonics CCD camera, and processed using the Metamorph digital imaging software (Siddiqui et al., Am J Physiol Lung Cell Mol Physiol 286, L1016-26 (2004)).

Expression of human wild type c-Met cDNA in C. elegans transgenic animals induced developmental abnormalities such as vulva defects, uncoordinated locomotion, and effects on viability of the progeny. A range of abnormalities in vulval development were observed such as vulvaless, multi-vulva, protruding vulva and ectopic vulva phenotypes. Similarly, observed locomotion defects included a variety of defects, ranging from mild defects in backward movement to severe defects in locomotion. In order to confirm that transgenic animals with abnormal phenotypes expressed the human c-met gene, c-Met expression was examined at the protein level by immunoblotting whole animal protein extracts against antibodies specific to human c-Met protein; furthermore, PCR reactions were performed, utilizing human c-Met specific primers targeting the juxtamembrane domain. A wide variety of phenotypes were observed in the transgenic animals overexpressing human c-Met both in the presence and absence of nicotene, such as growth arrest, uncoordinated locomotion, abnormal vulval formation, and abnormal body shapes and sizes (see Table 12 below).

In order to determine the role of c-Met juxtamembrane domain mutations found in human lung cancer, transgenic animals were generated by injecting mutant R988C cDNA and T1010I cDNA into the wild type N2 C. elegans worms. The wild type human c-Met injected animals showed an increased frequency of abnormal phenotypes when compared to wild type N2 worms, such as growth arrest in 6% (p<0.0001), paralysis in 3.32% (p=0.0018), locomotion defects in 4.87% (p<0.0001), and altered body morphology in 3.05% (p=0.0037). However, when the c-Met R988C cDNA construct was injected, there was a significant increase in the observed defects in transgenic animals, such as an increase in growth arrest rate to 9% (from 6% in wild-type, p<0.0001) as well as an increase in paralysis rate to 7.4% (from 3.32% in wild-type, p<0.0001). Similarly, an increase in the rate of locomotion defects to 8.12% (from 4.87% in wild-type, p<0.0001), and a significant increase in body shape changes (9.02%, from 3.05% in wild-type, p<0.0001) was observed.

TABLE 12
Effects of nicotine on viability indices and vulval defects in C. elegans for c-Met and mutant c-Met

| | Without Nicotene | | | | Chronic Exposure with Nicotene (20 μm) | | | |
|---|---|---|---|---|---|---|---|---|
| | | Wt | | | | Wt | | |
| | N2 | c-Met | R988C | T1010I | N2 | c-Met | R988C | T1010I |
| Growth | 0 | 6 ± 0.76* | 9 ± 1.06* | 17.86 ± 0.99* | 1.23 ± 0.19** | 9.72 ± 0.16** | 22.9 ± 0.41** | 29.0 ± 0.11** |
| Arrest | | | | | | | | |
| Paralysis | 0 | 3.32 ± 0.7* | 7.4 ± 0.21* | 7.52 ± 0.39* | 2 ± 0.02** | 8 ± 0.08** | 19 ± 0.8** | 22.4 ± 1.09** |
| Locomotion | 0 | 4.87 ± 0.06* | 8.12 ± 0.37* | 26 ± 0.34* | 1 ± 0.18** | 11 ± 0.09** | 15 ± 0.07** | 33 ± 1.8** |
| Defects | | | | | | | | |
| Body | 0 | 3.05 ± 0.09* | 9.02 ± 0.11* | 13.7 ± 1.0* | 3 ± 0.21** | 12 ± 0.22** | 18 ± 0.13** | 21 ± 0.14** |
| Shape | | | | | | | | |
| Changes | | | | | | | | |

All values are mean % ± s.e.m for three different experiments (total n = 300 in each group).
*= values obtained differ significantly from control N2 without nicotine exposure.
**= values obtained differ significantly from similar gorup without nicotine exposure

The growth arrest phenotypes of T1010I c-Met expressing transgenic animals showed an even a higher percentage of defects as compared to the R988C c-Met or the wild type c-Met expressing transgenic worms. In the T1010I transgenic animals growth arrest was observed in 17.86% of the progeny, complete paralysis was observed in 7.52% and locomotion defects were seen in 26% of transgenic animals. Body shape changes were observed in 13.7% transgenic animals (all comparisons to wild-type worms had p<0.0001).

The most profound effect of c-Met mutations was observed in viability of the transgenic animals. Human wild type c-Met over-expressing transgenic animals showed 70% (p<0.0001, compared to wild-type worms) viability whereas R988C c-Met transgenic animals showed 47% viability and T1010I transgenic animals had an extremely low viability rate at about 9%. (N2 wild type animals were 98.5% viable). Similar to the effect of c-Met expression on animal viability, vulval formation defects also followed a similar trend. Wild-type N2 controls had no significant rate of vulval defects; transgenic animals, however, showed a high incidence of abnormal (p<0.0001) vulval cell lineages: 1.53% of wild-type c-Met over-expressing animals had vulval defects (p=0.1237). The number of animals showing abnormal vulval cell lineages increased to 10% for R988C mutants and 15.12% for the T1010I transgenic animals (p<0.0001).

Nicotine Exposure of C. elegans

To determine whether a c-Met germline mutation alone is sufficient for lung cancer development or whether in order to develop lung cancer development a synergism of c-Met with smoking is necessary, the C. elegans transgenic animals described above were exposed to nicotine. Nicotine response experiments were performed in M9 buffer as previously described by Waggoner, et al. (Waggoner et al., J Neurosci 20, 8802-11 (2000)). Animals were cultured at 20° C. on nematode growth media (NGM) plates seeded with E. coli strain OP50. To examine the nicotine dose response, individual young adult hermaphrodites were placed in 6-well microtiter plate in M9 buffer at different concentrations of nicotine. Following one hour incubation with nicotine medium plates at 20° C., total numbers of eggs laid by each individual animal were counted.

An increase in vulval developmental defects and other morphological phenotypes were observed when c-Met transgenic animals were chronically exposed to nicotine (20 μM) for 24 hours or more. The c-Met transgenic animals showed a higher incidence of vulval defects, when compared to N2 controls. In nicotine-exposed N2 animals, vulval defects were observed in 2% of the animals. Rates of vulva defects in transgenic animals were, however, much higher: 6% in wild type c-Met (p=0.02), 15% in R988C animals (p<0.0001) and 28% in T1010I animals (p<0.0001). Similarly, viability in the presence of nicotine was 91.94% for N2 wild-type animals, whereas wild-type c-Met injected transgenic animals exposed to nicotine had only 65.06% viability, and only 30.54% and 4.58% viability for R988C and T1010I mutants, respectively (p<0.0001 for all comparisons). In addition to vulval defects, transgenic animals exposed to nicotine showed considerable increase in other developmental abnormalities such as growth arrest, locomotion defects and altered body morphologies. 1.23% of N2 animals showed growth arrest when exposed to nicotine; in transgenic animals exposed to nicotine, 9.72% of wild type human c-Met carriers had growth-arrested animals (p=0.0007), whereas 22.9% (p<0.0001) and 29% (p<0.0001) of R988C and T1010I mutants had growth arrest, respectively. Similarly, the prevalence of locomotion defects was: N2, 1%; 11% in wild type c-Met, 15% in R988C, and 33% in T1010I transgenic animals (p<0.0001 for all comparisons). In addition to animals showing locomotion defects, a distinct phenotype of animals showing complete paralysis was observed, which was similarly augmented by nicotine in wild type and mutant c-Met transgenic animals: N2 2%, wild type c-Met 8% (p=0.0011), 19% for R988C (p<0.0001) and 22.4% (p<0.0001) for T1010I transgenic animals. In addition, exposure to nicotine also resulted in transgenic animals showing bizarre body morphologies, such as dumpy, small, roller and long animals (Brenner, Genetics 77, 71-94 (1974)). Body morphology changes were observed in 3% N2 control animals; 12% in wild type c-Met, 18% in R988C, and 21% in T1010I transgenic animals (p<0.0001 for all comparisons).

These results indicate that human c-Met juxtamembrane mutations R988C and T1010I result in higher incidence of developmental defects as well as reduced animal viability, and these abnormalities can be augmented by exposure to nicotine. Even though there is a strong dose-dependent association between lung cancer and cigarette smoking, the majority of smokers don't develop lung cancer. It is possible, based on Knudson's two-hit hypothesis, that there is a hereditary component to developing lung cancer; smoking could, therefore, increase the chances of high-risk individuals developing lung malignancies. There has been a report of a specific germline EGFR mutation that may predispose to the development of lung cancer (Bell et al., Nat Genet. 37, 1315-6 (2005). In C. elegans, chronic exposure to nicotinic receptor agonists such as nicotine is known to induce adaptive changes in nicotinic acetylcholine receptor (nAChR) function and abundance (Schafer, J Neurobiol 53, 535-41 (2002)). Initial application of exogenous nicotine causes nematode body muscles to contract; however, after a prolonged exposure to nicotine, animals develop tolerance and are able to move easily in the presence of agonist concentrations that would paralyze a naive worm (Lewis et al., Neuroscience 5, 967-89, 1980). Also, when nicotine-tolerant animals are placed on a nicotine-free growth plates, these animals show uncoordinated locomotion, suggesting that nematodes can have both nicotine tolerance as well as nicotine dependence. Waggoner et al. observed nicotine tolerance in the C. elegans egg laying behavior. Chronic exposure of animals to nicotine resulted in tolerant animals becoming insensitive to stimulation of egg-laying by nicotine agonists such as levamisole. These data indicate that exposure to nicotine synergizes with the deleterious effects of c-Met mutation on C. elegans development at several levels: animal viability, vulval development, growth arrest, paralysis, locomotion and body shape.

In summary, the C. elegans model described above shows that c-Met mutants can lead to altered phenotype and synergize with nicotine, thereby, ultimately reflecting the pathogenesis for lung cancer in patients with c-Met germline mutations.

### Example 15

In normal skin, c-Met is present on epithelial cells and melanocytes while HGF is produced mainly by mesenchymal cells and consequently, interacts with its receptor in a paracrine manner (Hsu et al., Differentiation, 70: 522-36 (2002)). HGF is a mitogen of human melanocytes and overexpression of c-Met correlates with the invasive growth phase of melanoma cells. Recent studies by Herlyn's group have shown that most of melanoma cells, but not normal melanocytes, produce HGF, which can induce sustained activation of its receptor. Hence, an autocrine HGF/c-Met signaling loop may be involved in the development of melanomas. Consistent with this, prolonged HGF stimulation induces the downregulation of the intercellular adhesive molecule E-cadherin that is implicated in the control of melanocyte proliferation. Finally, in transgenic mice that ubiquitously expressed HGF, ectopic localization of melanocytes and hyperpigmentation in skin were observed and melanoma arose spontaneously. In these mice, ultraviolet radiation-induced carcinogenesis was accelerated (Noonan et al., Cancer Res, 60: 3738-43 (2000)). It is suggested that c-Met autocrine activation induced development of malignant melanoma and acquisition of the metastatic phenotype (Otsuka et al., Cancer Res, 58: 5157-67 (1998)).

### Materials and Methods

### Reagents and Antibodies

SU11274: [(3Z)-N-(3-chlorophenyl)-3-({3,5-dimethyl-4-[(4-methylpiperazin-1-yl)carbonyl]-1H-pyrrol-2-yl}methylene)-N-methyl-2-oxo-2,3-dihydro-1H-indole-5-sulfonamide] (obtained from Pfizer Inc, San Diego, Calif.) was suspended in DMSO and kept in small aliquots at −20 degrees. Phosphospecific antibody to pY1230/1234/1235 (autophosphorylation site) was obtained from Biosource International, Camarillo, Calif. Phosphospecific antibodies for pS473 on AKT and pT421/pS424 on p70 S6-Kinase were obtained from Cell Signaling, Beverly, Mass. Total c-Met and BCL_{XL} antibodies were obtained from Santa Cruz Biotechnology, Santa Cruz, Calif. and β-actin from Sigma Aldrich (St. Louis, Mo.). For detecting melanoma differentiation proteins the following antibodies were used anti-tyrosinase monoclonal antibody (clone T311, Nova-castra, New Castle upon Tyne, UK), anti-gp-100 monoclonal antibody (MO 634, Dako corporation, Carpinteria, Calif.) and anti-MART-1/Melan-A monoclonal antibody (clone M2-7C10, Signet, Dedham, Mass.).

### Cell Lines and Cell Culture and Cell Cycle Anaylsis

MM-AN, MU, PM-WK, MM-RU, MM-MC, MM-LH and RPM-EP melanoma cells were grown in MEM (Cellgro, Hernon, Va.) with 10% FBS (Gemini Bioproducts, Woodland, Calif.). Triplicate melanoma cell cultures were grown in MEM with 10% FBS and treated with a final concentration of 5-10 μM of SU11274 or an equal volume of diluent (DMSO) as a control for 96 hrs. Cells were collected, stained with propidium iodide and analyzed with a Becton Dickinson FACS Scan and Cell Quest software.

### Preparation of Cell Lysates and Immunoblotting

Cells were lysed in lysis buffer containing 20 mM Tris, pH 8.0; 150 mM NaCl, 10% glycerol, 1% NP40, 0.42% NaF, 1 mM PMSF, 1 mM Na₃V0₄, 10 μl/ml Protease inhibitor cocktail (Sigma Aldrich, St. Louis, Mo.) as described previously (Maulik et al., Clin Cancer Res, 8: 620-7 (2002)). Cell lysates were separated by 7.5% or 10% SDS-PAGE electrophoresis under reducing conditions. Proteins were transferred to an immobilization membrane (Bio-Rad Laboratories, Hercules, Calif.) and immunoblotted using the enhanced chemiluminescence technique (PerkinElmer Life and analytical Sciences, Torrence, Calif.).

### Cell Growth Analysis

For measuring the effect of SU11274 on growth of melanoma cells, MM-AN, MU, PM-WK, MM-RU, MM-MC and RPM-EP cells were plated at 6×10⁴ cells in 60 mm dishes in MEM with 10% serum. After 24 hrs different concentrations of SU11274 were added and after 96 hrs cells were trypsinized and counted in a cell counter (Beckman Coulter). Each data point was repeated in triplicate.

Specific Phosphorylation of c-Met and Other Signaling Molecules Via HGF and Inhibition with SU11274

MU and MC cells that expressed c-Met were deprived of growth factors by incubation in serum free medium containing 0.5% BSA for 24 hours with or without SU11274 (5 μM). After treatment with or without SU11274 in serum free medium, cells were stimulated with or without HGF (EMD Biosciences, San Diego, Calif.) at 40 ng/ml for 7.5 minutes at 37° C. After harvesting, the cells were subjected to the standard procedures of immunoblotting. Antibodies specifically against phosphorylated c-Met, and other proteins are as described above.

### Reactive Oxygen Species Measurement by Immunofluorescence

5×10⁴ MU melanoma cells were plated in 35 mm petri dishes. After 24 hrs MU Melanoma cells were kept in serum free media containing 0.5% BSA with and without 5 μM SU11274 for 12 hrs. Cells were treated with 40 ng/ml HGF for 7.5 min, then treated with DHE 10 μM (molecular probes Inc, Eugene, Oreg.) for 30 min, after which they were visualized under a fluorescent Olympus microscope IX81, 20× objective, ND3 and Rhodamine filter sets. Fluorescence was then quantified using Image J and graphed.

siRNA and Transfection

siRNA against c-Met was obtained from Dharmacon (Lafayette, Colo.). Briefly four pooled SiRNA duplexes were transiently transfected into MU melanoma cells by the Dharmacon protocol utilizing Oligofectamine obtained from Invitrogen (Carlsbad, Calif.). Mock transfection was done in parallel using SignalSilence control SiRNA (Cell Signaling Technology) as negative control. For studies on the effect of Si RNA on differentiation proteins MU melanoma cells were transfected with Si RNA as described above and cell lysates were prepared after 12 hrs and immunoblotting performed as described above.

### Immunohistochemistry

For immunohistochemistry, paraffin-embedded, formalin-fixed tissues from patients with 20 nevi (4 intradermal, 2 junctional, 14 compound), 16 primary melanomas (5 superficial-spreading type, 9 nodular, 2 spindle), and 24 metastatic melanomas were analyzed. Immunostaining procedures were as described by Ma et al. (Ma et al., Cancer Res, 65: 1479-88 (2005)). For total c-Met immunostaining, 3D4 mouse monoclonal antibody from Zymed was used, and phospho-Met [pY1003] from Biosource was used. Appropriate negative controls for the immunostaining were prepared by omitting the primary antibody step and substituting it with non-immune rabbit serum.

All of the slides were reviewed and scored by two pathologists independently. For each case, the most representative tumor section was examined at 200× magnification to identify the percentage of tumor cells with c-Met expression.

### Cell Lines and DNA Sequence Analysis

Genomic DNA was isolated from 5 melanoma cell lines using a QIAamp DNA mini kit (QIAGEN, Valencia, Calif.). Melanoma tumors were obtained from pathology archives at the University of Chicago Hospital with institutional approved IRB protocol and tumor DNA was isolated using Proteinase K (0.03 mAU) from QIAGEN overnight at 56° C. The Sema domain, Juxtamembrane domain (JM) and Tyrosine kinase domain (TK) in c-Met genomic DNA were sequenced using standard PCR and sequencing techniques. Each PCR reaction contained 50 ng/ml of DNA, 1× Platinum Taq buffer, 1 mM dNTPs, 2.5 mM MgCl₂, 0.5 U Platinum Taq enzyme (Invitrogen, Carlsbad, Calif.) and 0.2 μM forward and reverse primers in a 20 μl reaction volume. The resulting PCR products were purified using a Qiaquick PCR purification kit (Qiagen, Valencia, Calif.). Sequencing fragments were detected using ABI Prism DNA Analyzer 3730XL (Applied Biosystems) and chemistry used was big dye version 3. The nucleotide position numbering was relative to the first base of the translational initiation codon according to full-length human c-Met cDNA (Ma et al., Cancer Res, 63: 6272-81 (2003) and Ma et al., Cancer Res, 65: 1479-88 (2005)).

### Results

Expression of c-Met in Melanoma Cell Lines

The expression of c-Met RTK protein was studied in seven melanoma cell lines. It was found that the 140-KDa β subunit of c-Met was expressed in all seven melanoma cell lines and MM-AN melanoma cells expressed minimal levels of c-MET in comparison to other melanoma cell lines.

### Effect of SU11274 on the Proliferation and Apoptosis of Melanoma Cell Lines

SU11274, which is a highly specific inhibitor of c-Met, was tested on the six melanoma cell lines. In all melanoma cell lines that expressed c-Met, the IC₅₀ of SU11274 was between 1 to 2.5 μM, suggesting inhibition of c-Met with the specific small molecule inhibitor SU11274 which inhibits growth of melanoma cells. The IC₅₀ of MM-AN which expresses minimal levels of c-MET was found to be 5 μM, 2 fold higher than that of cells lines which expressed abundant levels of c-Met. Apoptosis was seen in all five melanoma cell lines which expressed c-Met. By 96 hrs between 12 to 58% of cells in different melanoma cell lines appeared in the sub G₀/G₁ region of the FACS profile indicating apoptosis. No apoptosis could be detected in MM-AN cells.

Novel c-Met Mutations in Melanoma

c-Met mutations in lung cancer was recently described by Ma et al. (Cancer Res; 63: 6272-81 (2003)) and in this study it was evaluated whether similar mutations could also be found in melanoma. Melanoma cell lines MM-AN, PM-WK, MM-RU, MM-MC, and RPM-EP were analyzed for mutations in the “hot-spots” of c-Met. A new missense heterozygous mutation N948S was found in exon 13 in the Juxtamembrane (JM) domain in PM-WK, MM-RU and MM-MC. Specifically, a point mutation 2843 A>G was shown to result in a missense substitution from asparagine to serine at codon 948.

Fourteen melanoma tumors were analyzed and a novel mutation R988C in exon 14 in the JM domain was discovered. Specifically, a point mutation 2962 C>T resulted in missense substitution from arginine to cysteine. This mutation in the JM domain has also been seen in non-small cell and small cell lung cancer tumors (Ma et al., Cancer Res; 63: 6272-81 (2003) and Ma et al., Cancer Res 65: 1479-88 (2005)) however this is the first report of a mutation in c-Met in human melanoma tumors. Clustering of mutations in the JM domain of c-Met have been linked to induced cytoskeletal changes and increase in tumorigenicity (Ma et al., Cancer Res; 63: 6272-81 (2003)). There were no mutations found in the SEMA or catalytic tyrosine kinase domain.

### Effect of SU11274 on Differentiation of Melanoma Cell Lines

After treatment of MM-RU, MU and MM-MC melanoma cell lines for 72 hrs with 5 μM SU11274 it was noted that all three cell lines exhibited a change in morphology, with a dendritic and differentiated appearance similar to normal human melanocytes (20). MU melanoma cells, which are melanized spindle shaped or tripolar cells became very dendritic and differentiated on exposure to SU11274. MM-RU, MM-MC which are less differentiated, amelanotic, round or polygonal cells became bipolar, tripolar or multipolar and phenotypically resembled the normal human melanocyte. Thus SU11274 could induce a differentiated phenotype in a wide variety of melanoma cells melanized, amelanotic, less and more differentiated melanoma cells.

It was hypothesized that SU11274 could not only induce a differentiated phenotype, but also MITF, tyrosinase and other melanocyte associated differentiation markers. By immunoblotting 12 hrs after treatment with SU11274 an increase in levels of MITF (3 fold) was seen with a concomitant increase in the levels of melanocyte associated antigens tyrosinase (12 fold), gp-100 (6 fold) and MART-1 (2 fold). The increase in the level of various differentiation proteins was determined by densiometric anaylsis after treatment with 10 μM SU11274.

Si RNA Against c-Met Inhibits Growth and Induces Differentiation of Melanoma Cells

The effect of si RNA against c-Met was also studied on the proliferation and differentiation of MU melanoma cells. It was found that c-Met protein expression was downregulated (47%) 72 hrs after treatment with si RNA. Further it was found that 72 and 96 hrs after transfection with oligofectamine the proliferation of MU melanoma cells was reduced by 60% and 56% respectively. SU11274 at 5 μM and 10 μM inhibited the growth of melanoma cells by 72% and 88% respectively. The inhibitory effect of SU11274 was more pronounced than si RNA since SU11274 is a potent and selective inhibitor of c-Met activity and function. SU11274 also inhibits c-Met signal transduction, particularly AKT which is necessary for cell survival and growth (Sattler et al., Cancer Res, 63: 5462-9, 22 (2003)) and Berthou et al., Oncogene, 23: 5387-93 (2004). It was found that Si RNA could mimic the effect of SU11274 on differentiation and also induce a differentiated and dendritic phenotype similar to normal human melanocytes in MU melanoma cells. Similar to SU11274 c-Met Si RNA 12 hrs after transfection induced an increase in MITF (4 fold) and several of the melanocyte differentiation antigens tyrosinase (4-fold), gp-100 (2-fold) and MART-1 (2-fold) which have been targets for immunotherapy. The increase in the level of various differentiation proteins was determined by densiometric anaylsis after treatment with c-Met si RNA.

Stimulation by HGF Leads to an Increase in ROS However Treatment with SU11274 Decreases ROS in MU Melanoma Cells.

The endogenous levels of reactive oxygen species (ROS) was also analyzed in MU melanoma cells with and without HGF treatment using DHE by fluorescence microscopy. DHE is a redox sensitive probe that has widely been used to detect intracellular superoxide anion. The relative levels of ROS were increased in response to HGF by 47%, and SU11274 decreased the ROS response to HGF to 3%. Since an ROS response is necessary for the survival of cancer cells these results indicate that lowering ROS responses considerably by SU11274 could be used as a potential chemotherapeutic agent.

SU11274 Prevented Tyrosine Phosphorylation of c-Met in MU Melanoma Cells and Blocked c-Met Dependent Signaling Events.

The ability of SU11274 to inhibit activation of c-Met was examined by immunoblotting. Treatment with HGF increased the autophosphorylation of c-MET at the activation loop site phospho-epitope [pY1230/1234/1235]. SU11274 completely abolished the phosphorylation of the above tyrosine residues at the activation site. HGF binding to c-Met activated its' tyrosine kinase, AKT (S-473) and S-6 kinase (T-421/S424). At 5 μM SU11274 completely inhibited HGF induced phosphorylation of c-Met, AKT and S6 kinase. Additionally SU11274 downregulated BCL_{XL}, an inhibitor of apoptosis indicating that SU11274 stimulates apoptosis as was seen earlier. No effect of the c-Met mutation in MM-MC was found on the intrinsic c-Met kinase activity or downstream signaling events in comparison to MU which does not have a c-Met mutation. In addition, SU11274 inhibited phosphorylation of c-Met and AKT in both MM-MC and MU indicating that c-Met inhibitors could also be therapeutically effective in individuals with melanoma with c-Met mutations

Expression of c-Met and Activated Phospho-Met in Melanoma.

Analysis of the frequency and localization of c-Met expression by immunohistochemical staining in nevi and melanomas was also performed. Paraffin-embedded, formalin fixed tissues from 20 patients with nevi and 40 with malignant melanomas were analyzed. Both total c-Met and activated phospho-Met [pY1003] were demonstrated in paraffin embedded formalin fixed melanoma tissues using IHC techniques. c-Met was negative in 85% of nevi, focally positive in 3 compound nevi (15% of cases), and positive in 88% of melanomas examined. In primary melanomas the vast majority of cases (87.5%) showed cytoplasmic staining. In metastatic melanomas 29% cases exhibited cytoplasmic positivity, and 58.5% both cytoplasmic and membranous staining. Thus, primary melanomas exhibited cytoplasmic positivity, whereas metastatic melanomas showed both cytoplasmic and membranous pattern of c-Met staining. Interestingly preferential expression of phospho-Met was located in the invasive front of melanoma. In addition to expression of c-Met, expression of phospho-Met [pY1003] showed that c-Met was activated at phospho-epitope [pY1003] in 21% of human melanoma indicating that activation of c-Met can occur in melanoma. Expression of activated c-Met at phospho-epitopes [pY1003] was not detected in normal epidermis or nevi.

### Discussion

The studies of RTKs in solid tumors have come to fruition as molecularly targeted therapy (Lydon et al., Leuk Res, 28 Suppl 1: S29-38 (2004)). These studies show that HGF/c-Met pathway is functional in melanoma and this can be a useful target for therapeutic intervention. c-Met was functionally expressed in all melanoma cell lines and most tumor tissues had unique preferential expression at the tumor invasive front. Targeted inhibition of c-Met either via SU11274 or specific siRNA led to decreased cell growth and viability. SU11274 inhibition of c-Met abrogated tyrosine phosphorylation of cellular proteins including c-Met itself as well as its downstream signaling proteins.

SU11274 and c-Met siRNA were also able to induce MITF, several melanoma differentiation proteins and a phenotype similar to normal human melanocytes. Stimulation of ROS by HGF led to increased ROS formation which was completely inhibited by SU11274. Lastly unique c-Met alterations in the JM domain were identified in melanoma cell lines and tumor tissue. It has been shown by Ma et al., that the JM domain has a novel role in c-Met signaling, motility, tumorigenicity and migration (Cancer Res, 63: 6272-81 (2003)). The novel c-Met mutations reported in this study are the first to be reported in melanoma.

This is the first report of the effect of a small molecule inhibitor SU11274 on human melanoma. Small molecule inhibitors specifically targeting c-Met represent an attractive novel targeted therapeutic approach. Met kinase autophosphorylation was reduced on sites that have been shown to be important for activation of pathways involved in cell growth and survival especially the phosphatidyinositol-3′-kinase (PI3K) pathway which is responsible for events such as proliferation, reduced apoptosis and anchorage independence (Rameh et al., J Biol Chem, 274: 8347-50, 26 (1999) and Toker et al., Mol Pharmacol, 57: 652-8 (2000)). SU11274 decreases phosphorylation of AKT which is downstream of PI3K and inhibits an anti-apoptotic protein BCL_{XL} which is downstream of AKT. Other strategies to inhibit c-Met reported are NK4 HGF (truncated form of HGF), peptide inhibition, antibody inhibition, Si RNA and ribozymes (Abounader et al., J Natl Cancer Inst, 9: 1548-56 (1999); Michieli et al., Oncogene, 18: 5221-31 (1999); Michieli et al., Cancer Cell, 6: 61-73 (2004); Shinomiya et al., Cancer Res, 64: 7962-70 (2004); and Wickramasinghe et al., Cell Cycle, 4: 683-5 (2005)). Inhibition of c-Met by Si RNA represents a novel and powerful strategy. Here, it was determined that treatment with Si RNA lowered c-Met protein expression and inhibited viability of melanoma cells considerably. Both c-Met Si RNA and SU11274 caused differentiation of melanoma cells, induced the transcription factor MITF which regulates expression of major melanoma associated differentiation proteins such as tyrosinase, TRP-1 and TRP-2 (Goding et al., Genes Dev, 14: 1712-28, 33 (2000) and Park et al., J Invest Dermatol, 119: 1218-9 (2002)). Melanoma differentiation is also associated with slower cell proliferation (Puri et al., Faseb J, 18: 1373-81 (2004)). Induced differentiation could be especially helpful in immunotherapy of melanoma when SU11274 or Si RNA induced increase in the expression of several differentiation antigens such as tyrosinase, gp-100 and MART-1, can potentially permit targeting of melanoma by sensitized T-lymphocytes.

In this study it was also determined that c-Met is expressed in cell lines and tumor tissues. This is the first study where activated c-Met has been detected in tumor tissue in melanoma. c-Met activation could happen as a result of overexpression, activating mutations or gene amplification. In addition, a 2-fold increase in the expression of phosphorylated c-Met was seen in melanoma in comparison to nevi. A new missense N948S mutation was identified in melanoma cell lines and a novel mutation R988C was detected in melanoma tumor tissues in the JM domain. This is the first report of a mutations in c-Met in melanoma. Mutations in the JM domain in SCLC are activating, influencing cell transformation, anchorage-dependent proliferation, cytoskeletal functions and cell motility and migration.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A prognostic method comprising determining whether a sample from a subject comprises a mutation in a nucleic acid sequence encoding human c-Met, wherein the mutation results in an amino acid change at one or more of positions L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or or V1333I, wherein the method comprises contacting the sample with a nucleic acid.

2. A prognostic method comprising determining whether a cancer sample from a subject comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

3. A method of detecting cancer in a sample comprising determining whether the sample comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, PI1301S, and/or V1333I.

4. A method of detecting cancer in a sample comprising determining whether the sample comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

5. A method for distinguishing between non-cancerous and cancerous tissue, said method comprising determining whether a sample comprising the tissue comprises a mutation in a nucleic acid sequence encoding human c-Met, wherein the mutation results in an amino acid change at position L211W, T230M, S244P, L229F, F253S, 5323G, A347T, E355K, R359Q, M362T, M431V, N4541, S470L, I852F, N948S, S1058P, R988C, T1010I, Q1029E, Si 167N, T12751, P1300S, P1301S, and/or V13331, wherein detection of the mutation in the sample is indicative of presence of cancerous tissue.

6. A method for distinguishing between non-cancerous and cancerous tissue, said method comprising determining whether a sample comprising the tissue comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure, wherein detection of the mutation in the sample is indicative of presence of cancerous tissue.

7. A method of identifying a mutation in c-Met in a sample, said method comprising contacting a cancer sample from a subject diagnosed with cancer with an agent capable of detecting a mutation in a nucleic acid sequence encoding human c-Met, wherein the mutation results in an amino acid change at one or more of positions L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, or V1333I.

8. A method of identifying a mutation in c-Met in a cancer, said method comprising contacting a cancer sample with an agent capable of detecting a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

9. A method of identifying a cancer that is susceptible to treatment with a c-Met inhibitor, said method comprising determining whether a cancer sample from a subject comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N4541, S470L, I852F, N9485, S1058P, R988C, T1010I, Q1029E, S1167N, T1275I, P1300S, P1301S, and/or V1333I.

10. A method of identifying a cancer that is susceptible to treatment with a c-Met inhibitor, said method comprising determining whether a cancer sample from a subject comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure.

11. A method of determining responsiveness of a cancer in a subject to treatment with a c-Met inhibitor, said method comprising determining whether a cancer sample from a subject who has been treated with the c-Met inhibitor comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N4541, S470L, I852F, N948S, S1058P, T1010I, Q1029E, S1167N, T1275I, P1300S, P1301S, and/or V1333I, wherein absence of the mutated nucleic acid sequence is indicative that the cancer is responsive to treatment with the c-Met inhibitor.

12. A method of determining responsiveness of a cancer in a subject to treatment with a c-Met inhibitor, said method comprising determining whether a cancer sample from a subject who has been treated with the c-Met inhibitor comprises a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation affects exon splicing and/or protein structure, wherein absence of the mutated nucleic acid sequence is indicative that the cancer is responsive to treatment with the c-met inhibitor.

13. A method for monitoring minimal residual cancer in a subject treated for cancer with a c-Met inhibitor, said method comprising determining whether a sample from a subject who is treated with the c-Met inhibitor comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N454I, S470L, I852F, N948S, S1058P, R988C, T1010I, Q1029E, S1167N, T1275I, P1300S, P1301S, and/or V1333I, wherein detection of said mutation is indicative of presence of minimal residual cancer.

14. A method for monitoring minimal residual cancer in a subject treated for cancer with a c-Met inhibitor, said method comprising determining whether a sample from a subject who has been treated with the c-Met inhibitor comprises a mutation in a nucleic acid sequence encoding human c-Met, wherein the sequence is mutated in one or more of exons 2, 3, 4, 7, 9, 14, 15, 17, 19, 20 and/or 21, and/or their flanking introns, wherein the mutation affects exon splicing and/or protein structure, wherein detection of said mutation is indicative of presence of minimal residual cancer.

15. A method for amplification of a nucleic acid encoding human c-Met, wherein the nucleic acid comprises a mutation that results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I relative to wild type c-Met, said method comprising amplifying a sample suspected or known to comprise the nucleic acid with a nucleic acid comprising the sequence of any of the primers/probes listed in Tables 7-10.

16. A method for amplification of a nucleic acid encoding human c-Met, wherein the nucleic acid comprises a mutation in exons 2, 3, 4, 7, 9, 13, 14, 15, 17, 19, 20 and/or 21, and/or and/or their flanking introns, wherein the mutation affects exon splicing and/or protein structure, said method comprising amplifying a sample suspected or known to comprise the nucleic acid with a nucleic acid comprising the sequence of any of the primers/probes listed in Tables 7-10.

17. A method for identifying a specific mutation in c-met in a sample, wherein the mutation is one that results in an amino acid change at position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, and/or V1333I relative to wild type c-met, said method comprising contacting the sample with a nucleic acid comprising the sequence of any of the primers/probes listed in Tables 7-10.

18. A method for identifying a specific mutation in c-met in a sample, wherein the mutation is in exons 2, 3, 4, 7, 9, 13, 14, 15, 17, 19, 20 and/or 21, and/or their flanking introns, wherein the mutation affects exon splicing and/or protein structure, said method comprising contacting the sample with a nucleic acid comprising the sequence of any of the primers/probes listed in Tables 7-10.

19. A method of detecting presence of a mutated c-Met in a cancer, the method comprising contacting a sample suspected or known to comprise mutated c-Met with a nucleic acid comprising the sequence of any of the primers/probes listed in Tables 7-10.

20. A method of detecting the presence of a mutated c-Met in a cancer, the method comprising contacting a sample suspected or known to comprise mutated c-Met with an antigen binding agent capable of binding to a peptide that contains an amino acid change at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, P1301S, and/or V1333I relative to wild type c-Met, wherein binding of the agent or lack thereof, is indicative of presence or absence of a c-Met polypeptide comprising a mutation of at least a portion of exons 2, 3, 4, 7, 9, 13, 14, 15, 17, 19, 20 and/or 21.

21. A method for detecting a cancerous disease state in a tissue, said method comprising determining whether a sample from a subject suspected of having a cancer comprises a mutation in a nucleic acid sequence encoding human c-Met, wherein the mutation results in an amino acid change at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T1275I, P1300S, P1301S, and/or V1333I, wherein detection of said mutation is indicative of presence of a cancerous disease state in the subject.

22. A method for detecting a cancerous disease state in a tissue, said method comprising determining whether a sample from a subject suspected of having a cancer comprises a mutation in a nucleic acid sequence encoding human c-Met, whether the sequence is mutated in exons 2, 3, 4, 7, 9, 13, 14, 15, 17, 19, 20 and/or 21, and/or their flanking introns, wherein the mutation affects exon splicing and/or protein structure, wherein detection of said mutation is indicative of presence of minimal residual cancer.

23. A cancer biomarker, wherein the biomarker comprises c-met comprising a mutation that results in an amino acid change at one or more of positions position L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, or V1333I.

24. A cancer biomarker, wherein the biomarker comprises c-met comprising a mutation in exons 2, 3, 4, 7, 9, 13, 14, 15, 17, 19, 20 and/or 21, and/or their flanking introns, wherein the mutation affects exon splicing and/or protein structure.

25. The biomarker of claim 23, wherein the biomarker is a nucleic acid molecule.

26. The biomarker of claim 24, wherein the biomarker is a nucleic acid molecule.

27. The biomarker of claim 23, wherein the biomarker is a polypeptide.

28. The biomarker of claim 24, wherein the biomarker is a polypeptide.

29. A cancer imaging agent, wherein the agent specifically binds c-Met comprising a mutation, wherein the agent binds a c-Met polypeptide comprising a mutation at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, P1301S, and/or V1333I of the protein, or wherein the agent binds a c-Met encoding nucleic acid comprising a mutation at a nucleic acid position corresponding to a change in amino acid at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, P1301S, and/or V1333I.

30. A cancer imaging agent, wherein the agent specifically binds c-Met polypeptide comprising a deletion of at least a portion of exons 2, 3, 4, 7, 9, 13, 14, 15, 17, 19,20 and/or 21, or wherein the agent specifically binds c-Met encoding nucleic acid that comprises a mutation in a sequence that encodes exons 2, 3, 4, 7, 9, 13, 14, 15, 17, 19, 20 and/or 21.

31. A polynucleotide capable of specifically hybridizing to c-Met encoding nucleic acid comprising a mutation at a nucleic acid position corresponding to a change in amino acid at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, P1301S, and/or V1333I.

32. A polynucleotide capable of specifically hybridizing to c-Met encoding nucleic acid that comprises a mutation in a sequence that encodes exons 2, 3, 4, 7, 9, 13, 14, 15, 17, 19, 20 and/or 21.

33. An antigen binding agent capable of specifically binding to a c-Met polypeptide comprising a mutation at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, P1301S, and/or V1333I.

34. An antigen binding agent capable of specifically binding to a c-Met polypeptide comprising a mutation in exons 2, 3, 4, 7, 9, 13, 14, 15, 17, 19, 20 and/or 21 that encode the c-Met polypeptide.

35. An array comprising polynucleotides capable of specifically hybridizing to c-Met encoding nucleic acid comprising a mutation at a nucleic acid position corresponding to a change in amino acid at position L211W, T230M, S244P, L229F, F253S, S323G, A347T, E355K, R359Q, M362T, M431V, N4541, S470L, 1852F, N948S, S1058P, R988C, T1010I, Q1029E, S1167N, T12751, P1300S, P1301S, and/or V1333I.

36. An array comprising polynucleotides capable of specifically hybridizing to a c-Met encoding nucleic acid that comprises a mutation in a sequence that encodes exons 2, 3, 4, 7, 9,13,14,15,17, 19, 20 andior 21.

37. A computer-readable medium comprising human c-Met amino acid polypeptide sequence comprising a mutation at position L211W, T230M, S244P, L229F, F253S, S323G, A347T, E355K, R359Q, M362T, M431V, N4541, S470L, I852F, N948S, 51058P, R988C, T1010I, Q1029E, S1167N, T12751, P1300S, P1301S, and/or V1333I, and/or nucleic acid sequence encoding a human c-Met polypeptide comprising a mutation at a nucleic acid position corresponding to a change in amino acid at position L211W, T230M, S244P, L229F, F253S, S323G, A347T, E355K, R359Q, M362T, M431V, N4541, S470L, I852F, N948S, S1058P, R988C, T1010I, Q1029E, S1167N, T12751, P1300S, P1301S, and/or V1333I.

38. A computer-readable medium comprising human c-Met amino acid polypeptide sequence comprising a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, and/or human c-Met encoding nucleic acid that a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns.

39. A kit comprising a composition of the invention, and instructions for using the composition to detect mutation in human c-Met at position L211W, T230M, S244P, L229F, F2535, 5323G. A347T, E355K, R359Q, M362T, M431V, N454I, S470L, 1852F, N948S, S1058P, R988C, T1010I, Q1029E, S1167N, T1275I, P1300S, P1301S, and/or V1333I.

40. A kit comprising a composition of the invention, and instructions for using the composition to detect human c-Met comprising a mutation in a nucleic acid sequence encoding human c-Met, wherein the sequence is mutated in one or more of exons 2, 3, 4, 7, 9, 13, 14, 15, 17, 19, 20 and/or 21, and/or their flanking introns, wherein the mutation 30 affects exon splicing and/or protein structure.

41. An isolated polynucleotide capable of encoding one or more polypeptides selected from the group consisting of SEQ ID NOs: 3-26, or a complement thereof.

42. An expression vector comprising a polynucleotide of claim 41, operably linked to an expression control sequence.

43. A host cell comprising an expression vector according to claim 42.

44. The host cell of claim 43, wherein the cell is a mammalian cell.

45. An isolated polypeptide comprising an amino acid sequence encoded by a polynucleotide of claim 41.

46. An isolated polypeptide comprising the sequence provided in SEQ ID NOS: 3-26, or a complement thereof.

47. An isolated polynucleotide capable of distinguishing between the sequence provided in SEQ ID NO: 1 or a complement thereof and a nucleic acid capable of encoding one or more polypeptides selected from the group consisting of SEQ ID NOs: 3-26.

48. A method of assessing a subject's increased susceptibility to develop a cancer comprising determining whether the sample comprises a mutation in a nucleic acid s equence encoding human c-Met, wherein the mutation results in an amino acid change at position L211W, T230M, S244P, L229F, F253S, S323G, A347T, E355K, R359Q, M362T, M431V,N4541, S470L, I852F,N948S, S1058P, R988C, T1010I, Q1029E, S1167N, T12751, P1300S, P1301S, and/or V1333I.

49. The method of claim 1, wherein the mutation affects exon splicing or protein structure.

50. The method of claim 1, wherein the subject has been diagnosed with a cancer.

51. The method of claim 50 wherein the cancer is lymphoma, leukemia, mycosis fungoide, carcinoma, adenocarcinoma, sarcoma, glioma, astrocytoma, blastoma, neuroblastoma, plasmacytoma, histiocytoma, melanoma, adenoma, hypoxic tumour, myeloma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, lung cancer, head and neck carcinoma, hematopoietic cancer, testicular cancer, colo-rectal cancer, prostatic cancer, or pancreatic cancer.

52. The method of claim 1, wherein the mutation in the nucleic acid sequence encoding human c-Met results is determined by a PCR amplification reaction.

53. The method of claim 52, wherein one or more primers selected from the primers listed in Tables 7-10 is used in the PCR amplification reaction.

54. The method of claim 7, wherein the mutation affects exon splicing and/or protein structure

55. The method of claim 55 wherein the cancer is lymphoma, leukemia, mycosis fungoide, carcinoma, adenocarcinoma, sarcoma, glioma, astrocytoma, blastoma, neuroblastoma, plasmacytoma, histiocytoma, melanoma, adenoma, hypoxic tumour, myeloma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, lung cancer, head and neck carcinoma, hematopoietic cancer, testicular cancer, colo-rectal cancer, prostatic cancer, or pancreatic cancer.

56. The method of claim 7, wherein the mutation in a nucleic acid sequence encoding human c-Met results in an amino acid change at one or more of positions L211W, T230M, S244P, L229F, F253S, S323G, A347T, E355K, R359Q, M362T, M431V, N454I, S470L, 1852F, N948S, S1058P, R988C, T1010I, Q1029E, S1167N, T1275I, P1300S, P1301S, or V1333I is determined by a PCR reaction.

57. The method of claim 56, wherein one or more primers selected from the primers listed in Tables 7-10 is used in the PCR amplification reaction.
